(19) United States
(12) Patent Application Publication
(10) Pub. No.: US 2018/0271908 A1
(43) Pub. Date: Sep. 27, 2018

(54) INTRAMUSCULAR ADMINISTRATON OF AUTOLOGOUS BONE MARROW FOR TREATMENT

(71) Applicant: CESCA THERAPEUTICS, INC., Rancho Cordova, CA (US)

(72) Inventors: Dalip SETHI, Rancho Cordova, CA (US); Bruce BAKER, Rancho Cordova, CA (US); Venkatesh PONEMONE, Rancho Cordova, CA (US); Kenneth Lee HARRIS, Los Angeles, CA (US); Markud D. LACHER, Moraga, CA (US); James HEISERMAN, Castro Valley, CA (US)

(21) Appl. No.: 15/571,244

(22) PCT Filed: Apr. 28, 2016
(86) PCT No.: PCT/US2016/029863

§ 371 Date: Nov. 1, 2017

Related U.S. Application Data
(60) Provisional application No. 62/156,126, filed on May 1, 2015.

(51) Int. Cl. A61K 35/16; A61K 35/18; A61K 35/28; A61K 45/06; A61K 9/00; A61P 9/10

(52) U.S. Cl. CPC A61K 35/16; A61K 35/18; A61P 9/10; A61K 45/06; A61K 9/0019; A61K 35/28

(57) ABSTRACT

Disclosed herein are methods of ameliorating or inhibiting critical limb ischemia or a condition associated with critical limb ischemia in a subject, whereby such methods comprise identifying a subject having a peripheral vascular disease, peripheral arterial disease, critical limb ischemia or a condition associated with critical limb ischemia and providing to said subject a composition comprising: a cell population, wherein the cell population comprises bone marrow total nucleated cells and red blood cells, an anticoagulant, and autologous plasma, wherein the composition has a viscosity of 1.5 to 5.0 centipoise (cP) measured at 37° C., wherein the composition comprises a viable cell dose and, wherein said composition is administered to said subject intramuscularly through a standard terminally-ported cannula needle, or a cannula side-ported needle or catheter comprising a plurality of ports and a closed end.

### INCORPORATION BY REFERENCE TO ANY PRIORITY APPLICATIONS

The present application claims priority to U.S. Provisional Application No. 62/156,126, filed on May 1, 2015. The content of this related application is hereby expressly incorporated by reference in its entirety.

### FIELD

The present disclosure relates to a method of treating, inhibiting or ameliorating critical limb ischemia or a condition associated with critical limb ischemia in a subject in need thereof and methods of delivering a composition comprising a viable cell population obtained from bone marrow, which can also include an anticoagulant, red blood cells, and autologous plasma.

### BACKGROUND

Critical limb ischemia (CLI) is a significant morbid disease that is seen more often in the aging population. Risk factors for CLI can be similar to those for atherosclerosis, which is hardening and narrowing of the arteries due to the buildup of fatty deposits. These factors can include, but are not limited to age, smoking, diabetes, being overweight or having severe obesity, a sedentary lifestyle, high cholesterol, high blood pressure and/or a family history of atherosclerosis or claudication. There are many approaches to the management of patients with CLI, due to the complexities that are involved with this debilitating disease. A thin line exists in the decision process between medical management versus surgical management, such as revascularization or amputation. Scoring systems, for example, can be helpful and can be used to predict outcomes in patients with CLI patients undergoing revascularization or amputation. However, new therapies for addressing the needs of people suffering from CLI are needed.

Critical limb ischemia is a term encompassing limb pain that occurs at rest, or impending limb loss that can be caused by a severe compromise of blood flow to the affected extremity. The hallmark of peripheral arterial occlusive disease is an inadequate blood flow to supply vital oxygen demanded by the limb. CLI occurs after chronic lack of blood supply, and over time this lack of blood supply can set off a cascade of pathophysiologic events that ultimately lead to rest pain or trophic lesions of the legs, or both. Thus, CLI is often considered the “end stage” of peripheral arterial disease (PAD).

Many in the field understand CLI to encompass any patient with chronic ischemic rest pain, ulcers, or gangrene attributable to objectively proved arterial occlusive disease. CLI can be considered as a disease process that occurs in a chronic setting of months to years and, if left untreated, can ultimately lead to limb loss secondary to lack of adequate blood flow and oxygenation through the distal extremities. Given that CLI is a severe manifestation of PAD, patients can be classified in the more severe ends of the Fontaine classification, a method by which peripheral artery disease is clinically classified, as stage III or stage IV.

Without aggressive or proper management, CLI can lead to amputations, however, there has also been recent advances in revascularization. Although amputation rates have declined, amputation still occurs due to late referrals to vascular surgeons or specialists late in the course of CLI. Additionally, there has been no agreed definition of a non-salvageable limb.

The treatment of CLI is dependent on the stage of CLI, of which the diagnosis is very important. Treatment can be nonsurgical management or surgical management. Nonsurgical management can include, but it is not limited to pain relief, local ulcer care and pressure relief, treatment of infection, spinal cord stimulation, and modification of atherosclerotic risk factors. Surgical managements can include revascularization or primary amputation.

Cell therapy such as adjuvant stem cell therapy can potentially be a promising treatment for CLI. Adjuvant therapy is a procedure in which secondary treatment is given at the same time, or immediately following, a primary therapy, in an effort to enhance treatment results. Adjuvant stem cell therapy can be efficacious in the repair and regeneration of tissues damaged from ischemic injury but more approaches to utilize stem cell therapy are needed.

### SUMMARY

New approaches for the delivery of a composition comprising stem cells, progenitor cells, red blood cells, and plasma to a subject in need thereof are disclosed herein. In some embodiments, methods for delivering such compositions utilize a technique that minimizes the shearing forces on the cells, which reduces the damage to the cells in the composition during delivery to the subject.

Some embodiments disclosed herein provide for methods of ameliorating or inhibiting critical limb ischemia or a condition associated with critical limb ischemia in a subject, whereby such methods comprise identifying a subject having a peripheral vascular disease, peripheral arterial disease, critical limb ischemia or a condition associated with critical limb ischemia; and administering intramuscularly to said subject a composition comprising: a cell population, wherein the cell population comprises bone marrow total nucleated cells and red blood cells, an anticoagulant, and autologous plasma, wherein the composition has a viscosity of 1.5 to 5.0 centipoise (cP) measured at 37° C., wherein the composition comprises a viable cell dose and, wherein said composition is administered through a standard terminally-ported cannula needle, or a cannula side-ported needle or catheter comprising a plurality of ports and a closed end. In some embodiments, said cell population comprises 2%, 3%, 4%, 5%, 6%, 7%, 8%, 9%, 10%, 11%, 12%, 13%, 14%, 15%, 16%, 17%, 18%, 19%, or 20% packed total cell fraction of the composition (total cell volume of the composition) or red blood cell fraction of the composition or an amount of total cells or red blood cells that is within a range defined by any two of the aforementioned values. In some embodiments, such packed cell fractions (total cell volume) or red blood cell fraction (% hematocrit) can be determined by microhematocrit centrifugation or other indirect methods. In some embodiments, the cell population comprises at least 1×10⁸ total nucleated cells (TNCs), and the cell population is processed from 120-180 mL bone marrow aspirate and is concentrated for a final 20 mL volume as enumerated intra-operatively using a rapid diagnostic instrument to guide the aspiration of at least 120 mL but no more than 180 mL of autologous bone marrow, wherein the cells comprise: a cell viability >70% and a white blood cell recovery >80%. In some embodiments, the cell population comprises CD34⁺ and CD34⁻ bone marrow stem cells. In some embodiments, the cell population comprises stromal cells. In some embodiments, these stromal cells are lineage negative/dim, CD45 negative/dim and CD73 positive cells. In some embodiments, the cell population comprises mesenchymal stem cells. In some embodiments, the cell population comprises hematopoietic stem cells, endothelial progenitor cells and CXCR4 positive cells. In some embodiments, the viable cell dose given to the subject, as measured after the bone marrow aspiration and after the bedside processing but prior to injection at the point of care is in the range of 1.75×10⁷ to 7.67×10⁷ white blood cells per mL. In some embodiments, the viable cell dose is, is about, or is at least, 1.75×10⁷, 2.0×10⁷, 3.0×10⁷, 4.0×10⁷, 5.0×10⁷, 6.0×10⁷, 7.0×10⁷ or 7.67×10⁷ white blood cells per ml, or is an amount within a range defined by any two of the aforementioned listed values. In some embodiments, the viable cell does is 4.0×10⁷ white blood cells per ml. In some embodiments, the viable cell dose given to the subject and as measured after the bedside processing is, is about, or is at least, 3.65×10⁶, 4.0×10⁶, 5.0×10⁶, 6.0×10⁶, 7.0×10⁶, 8.0×10⁶, 9.0×10⁶, 1.0×10⁷, 2.0×10⁷, or 2.15×10⁷ mononuclear cells per mL or an amount that is within a range defined by any two of the aforementioned listed values. In some embodiments, the viable cell dose given to the subject and as measured after the bedside processing is 1.08×107 mononuclear cells per mL. In some embodiments, the viable cell dose given to the subject and as measured after the bedside processing is, is about, or is at least, 5.0×10³, 6.0×10³, 7.0×10³, 8.0×10³, 9.0×10³, 1.0×10⁴, 2.0×10⁴, 3.0 1.0×10⁴, 4.0×10⁴, 5.0×10⁴, 6.0×10⁴, 7.0×10⁴, 8.0×10⁴, 9.0×10⁴, 1.0×10⁵, or 2.0×10⁵ colony forming units (CFU-H) per mL or is an amount within a range defined by any two of the aforementioned values. In some embodiments, the viable cell dose given to the subject and as measured after the bedside processing is 5.0×10³ to 20.0×10³ colony forming units (CFU-H) per mL. In some embodiments, said standard terminally-ported cannula needle, or cannula side-ported needle or catheter has a lumen size (diameter) of 0.33 mm. In some embodiments, said standard terminally-ported cannula needle, or cannula side-ported needle or catheter has a lumen size (diameter) of 0.51 mm. In some embodiments, said standard terminally-ported cannula needle, or cannula side-ported needle or catheter has a lumen size (diameter) of 0.69 mm. In some embodiments, said cannula side-ported needle or catheter comprises 10-24 side ports. In some embodiments, said cannula side-ported needle or catheter comprises, comprises about, comprises less than, or comprises more than, 10, 12, 14, 16, 18, 20, 22, or 24 side ports, or any number of side ports defined by any two of the aforementioned values. In some embodiments, said side ports have a diameter of 0.46 mm to 0.56 mm. In some embodiments, said side ports have a diameter that is, is about, is less than, or is more than, 0.46 mm, 0.48 mm, 0.50 mm, 0.52 mm, 0.54 mm, or 0.56 mm or any diameter within a range defined by any two of the aforementioned values. In some embodiments, said composition is administered to said subject at a rate of 0.1 mL to 0.5 mL per second. In some embodiments, said composition is administered to said subject at a rate that is, is about, is less than, 0.1 mL per second, 0.2 mL per second, 0.3 mL per second, to 0.4 mL per second, 0.5 mL per second, or any rate within a range defined by any two of the aforementioned values. In some embodiments, said composition is administered to said subject at a rate of 0.25 mL per second. In some embodiments, said administered composition has a muscle distribution area of 4-6 cm². In some embodiments, said administered composition has a muscle distribution area that is, is about, is less than, is more than, 4 cm², 5 cm², or 6 cm², or any muscle distribution area within a range defined by any two of the aforementioned values. In some embodiments, said composition is administered at a pressure of 3-5 psi. In some embodiments, said composition is administered at a pressure that is, is about, is less than, 3 psi, 4 psi or 5 psi or any pressure within a range defined by any two of the aforementioned values. In some embodiments, said composition is administered to said subject intramuscularly at a shear stress of less than or equal to 400 Pa. In some embodiments, said composition is administered to said subject intramuscularly at a shear stress of less than or equal to 300 Pa. In some embodiments, said composition is administered to said subject intramuscularly at a shear stress of less than or equal to 200 Pa. In some embodiments, said composition is administered to said subject intramuscularly at a shear stress of less than or equal to 150 Pa. In some embodiments, said composition is administered to said subject intramuscularly at a shear stress of less than or equal to 100 Pa. In some embodiments, the methods further comprise measuring in said subject an increased blood flow, an increase in perfusion, an increase in transcutaneous oxygen, an increase in angiogenesis, an increase in vascularity, an increase in limb salvage, or an increase in amputation-free survival rate after administration of said composition. In some embodiments, the methods further comprise measuring in said subject an increase in perfusion using transcutaneous oximetry after administration of said composition. In some embodiments, the methods further comprise measuring in said subject an increase in limb salvage after administration of said composition. In some embodiments, the methods further comprise measuring in said subject an increase in amputation-free survival rate after administration of said composition. In some embodiments, the methods further comprise measuring in said subject an improvement in index limb wound healing or closure.

Some embodiments disclosed herein provide methods for ameliorating or inhibiting critical limb ischemia or a condition associated with critical limb ischemia in a subject, wherein the methods comprise identifying a subject having a peripheral vascular disease, peripheral arterial disease, critical limb ischemia or a condition associated with critical limb ischemia; and administering intramuscularly or intradermally to said subject a composition comprising a cell population that comprises autologous bone marrow mononuclear cells and red blood cells (e.g., 2%-20% red cell fraction by volume or 2%-20% total cell fraction by volume), wherein said composition is aspirated, concentrated, and administered to said subject using a standard terminally-ported cannula needle, or a cannula side-ported needle or catheter comprising a plurality of ports and a closed end. In some embodiments, such packed cell fractions (total cell volume) or red blood cell fraction (% hematocrit) can be determined by microhematocrit centrifugation or other indirect methods. In some embodiments, the cannula side-ported needle or catheter has a lumen size (diameter) of 0.33 mm. In some embodiments, the cannula side-ported needle or catheter has a lumen size (diameter) of 0.51 mm. In some embodiments, the cannula side-ported needle or catheter has a lumen size (diameter) of 0.69 mm. In some embodiments, said cannula needle or catheter comprises 10-24 side ports. In some embodiments, said cannula side-ported needle or catheter comprises, comprises about, comprises less than, comprises more than, 10, 12, 14, 16, 18, 20, 22 or 24 side ports, or any amount of side ports defined by any two of the aforementioned values. In some embodiments, said side ports have a diameter of 0.46 mm to 0.56 mm. In some embodiments, said side ports have a diameter that is, is about, is less than, 0.46 mm, 0.48 mm, 0.50 mm, 0.52 mm, 0.54 mm, or 0.56 mm or any diameter within a range defined by any two of the aforementioned values. In some embodiments, said composition is administered to said subject at a dosage that is, is about, is less than, is more than, 0.25 mL to 0.5 mL per dose. In some embodiments, said composition is administered to said subject at 0.25 mL, 0.30 mL, 0.40 mL, or 0.5 mL per dose or any volume within a range defined by any two of the aforementioned values. In some embodiments, said composition is administered in a single dose. In some embodiments, said composition is administered in at least 30 doses. In some embodiments, said composition is administered in at least 40 doses. In some embodiments, said composition is administered in a single dose per day on each of multiple days. In some embodiments, said composition is administered in at least two doses per day on each of multiple days. In some embodiments, the methods further comprise measuring in said subject an increased blood flow, an increase in perfusion, an increase in transcutaneous oxygen, an increase in angiogenesis, an increase in vascularity, an increase in limb salvage, or an increase in amputation-free survival rate after administration of said composition. In some embodiments, the methods further comprise measuring in said subject an increase in perfusion using transcutaneous oximetry after administration of said composition. In some embodiments, the methods further comprise measuring in said subject an increase in limb salvage after administration of said composition. In some embodiments, the methods further comprise measuring in said subject an increase in amputation-free survival rate after administration of said composition. In some embodiments, the methods further comprise measuring in said subject an improvement in index limb wound healing or closure.

### BRIEF DESCRIPTION OF THE DRAWINGS

FIG. 1 shows a schematic illustration of an exemplary standard cannula needle.

FIG. 2 shows a schematic illustration of an exemplary cannula side ported needle.

FIG. 3 shows a computer-generated view of an exemplary cannula side ported needle carrying a Non-Newtonian mixed material model at a steady state, in which the shear stress and velocities are measured through the planes of the needle. The viscosities vary across the flow profile in the lumen length, proximal end to distal tip, and out the side ports.

FIGS. 4A and 4B show computer-generated velocity magnitude in an exemplary standard cannula needle carrying a Non-Newtonian mixed material model at two different flow rates of (A) Q1 (0.25 ml/sec) and (B) Q2 (0.5 ml/sec).

FIG. 5 shows computer-generated wall shear stress exhibited in an exemplary standard cannula needle at two rates of Q1 (0.25 ml/sec) and at Q2 (0.5 ml/sec).

FIG. 6 shows computer-generated shear strain rate using an exemplary standard cannula needle carrying a Non-Newtonian mixed material model at two rates of Q1 (0.25 ml/sec) and Q2 (0.5 ml/sec) in which the cross section of the tip of the needle is shown.

FIG. 7 shows computer-generated velocity vectors in an exemplary cannula side ported needle (TIN-21-5.0) carrying a Non-Newtonian mixed material model at a rate of Q1 (0.25 ml/sec) with port 1 being first and port 22 being last from proximal end to distal tip.

FIG. 8 shows the examination of computer-generated wall shear stress in an exemplary cannula side ported needle (TIN-21-5.0) carrying a Non-Newtonian mixed material model at a rate of Q1 (0.25 ml/sec) with port 1 being first and port 22 being last from proximal end to distal tip.

FIG. 9 shows computer-generated shear strain rate in an exemplary cannula side ported needle (TIN-21-5.0) carrying a Non-Newtonian mixed material model at a rate of Q1 (0.25 ml/sec) with port 1 being first and port 22 being last from proximal end to distal tip.

FIG. 10 shows computer-generated velocity vector measurement in an exemplary cannula side ported needle (TIN-19-7.5) carrying a Non-Newtonian mixed material model at a rate of Q1 (0.25 ml/sec) with port 1 being first and port 22 being last from proximal end to distal tip.

FIG. 11 shows computer-generated wall shear strain rate in an exemplary cannula side ported needle (TIN-19-7.5) carrying a Non-Newtonian mixed material model at a rate of Q1 (0.25 ml/sec) with port 1 being first and port 22 being last from proximal end to distal tip.

FIG. 12 shows computer-generated shear strain rate in an exemplary cannula side ported needle (TIN-19-7.5) carrying a Non-Newtonian mixed material model at a rate of Q1 (0.25 ml/sec) with port 1 being first and port 22 being last from proximal end to distal tip.

FIG. 13 shows computer-generated velocity vectors in an exemplary cannula side ported needle (TIN-19-10.0) carrying a Non-Newtonian mixed material model at a rate of Q1 (0.25 ml/sec) with port 1 being first and port 22 being last from proximal end to distal tip.

FIG. 14 shows computer-generated wall shear strain rate in an exemplary cannula side ported needle (TIN-19-10.0) carrying a Non-Newtonian mixed material model at a rate of Q1 (0.25 ml/sec) with port 1 being first and port 22 being last from proximal end to distal tip.

FIG. 15 shows computer-generated shear strain rate in an exemplary cannula side ported needle (TIN-19-10.0) carrying a Non-Newtonian mixed material model at a rate of Q1 (0.25 ml/sec) with port 1 being first and port 22 being last from proximal end to distal tip.

FIG. 16 shows computer-generated velocity vectors in an exemplary cannula side ported needle (TIN-21-5.0) carrying a Non-Newtonian mixed material model at a rate of Q2 (0.50 ml/sec) with port 1 being first and port 22 being last from proximal end to distal tip.

FIG. 17 shows computer-generated wall shear strain rate in an exemplary cannula side ported needle (TIN-21-5.0) carrying a Non-Newtonian mixed material model at a rate of Q2 (0.50 ml/sec) with port 1 being first and port 22 being last from proximal end to distal tip.

FIG. 18 shows computer-generated shear strain rate in an exemplary cannula side ported needle (TIN-21-5.0) carrying a Non-Newtonian mixed material model at a rate of Q2 (0.50 ml/sec) with port 1 being first and port 22 being last from proximal end to distal tip.

FIG. 19 shows computer-generated volumetric flow rate between the flow rates of Q1 and Q2 using an exemplary cannula side ported needle at different port numbers. TIN-21-5.0 was used as an example.

FIG. 20 shows computer-generated velocity vectors in an exemplary cannula side ported needle (TIN-19-7.5) carrying a Non-Newtonian mixed material model at a rate of Q2 (0.50 ml/sec) with port 1 being first and port 22 being last from proximal end to distal tip.

FIG. 21 shows computer-generated wall shear strain rate in an exemplary cannula side ported needle (TIN-19-7.5) carrying a Non-Newtonian mixed material model at a rate of Q2 (0.50 ml/sec) with port 1 being first and port 22 being last from proximal end to distal tip.

FIG. 22 shows computer-generated shear strain rate in an exemplary cannula side ported needle (TIN-19-7.5) carrying a Non-Newtonian mixed material model at a rate of Q2 (0.50 ml/sec) with port 1 being first and port 22 being last from proximal end to distal tip.

FIG. 23 shows computer-generated volumetric flow rate between the flow rates of Q1 and Q2 using an exemplary cannula side ported needle (TIN-19-7.5) at different port numbers with port 1 being first and port 22 being last from proximal end to distal tip.

FIG. 24 shows computer-generated velocity vectors in an exemplary cannula side ported needle (TIN-19-10) carrying a Non-Newtonian mixed material model at a rate of Q2 (0.50 ml/sec) with port 1 being first and port 22 being last from proximal end to distal tip.

FIG. 25 shows computer-generated wall shear strain rate in an exemplary cannula side ported needle (TIN-19-10) carrying a Non-Newtonian mixed material model at a rate of Q2 (0.50 ml/sec) with port 1 being first and port 22 being last from proximal end to distal tip.

FIG. 26 shows computer-generated shear strain rate in an exemplary cannula side ported needle (TIN-19-10) carrying a Non-Newtonian mixed material model at a rate of Q2 (0.50 ml/sec) with port 1 being first and port 22 being last from proximal end to distal tip.

FIG. 27 shows computer-generated volumetric flow rate between the flow rates of Q1 and Q2 using an exemplary cannula side ported needle (TIN-19-10) at different port numbers with port 1 being first and port 22 being last from proximal end to distal tip.

FIG. 28 shows computer-generated average wall shear stress over the side ports at the flow rates of Q1 and Q2 using an exemplary cannula side ported needle. TIN-19-10 was used as an example.

FIG. 29 shows a comparison of computer-generated velocity contours at Q1.

FIG. 30 shows a comparison of computer-generated velocity contours at Q2.

FIG. 31 shows a comparison of computer-generated shear wall stress at Q1.

FIG. 32 shows a comparison of computer-generated shear wall stress at Q2.

FIG. 33 shows the similarity of volume flow rate distribution among side ported cannula needles of different lumen sizes, diameter & length, at flow rates of Q1 and Q2, using the computer based modeling.

FIG. 34 shows WBC data in a graphical presentation. The data indicates no effect of shear on aBMC samples when passed through needle at a flow-rate of 0.25 mL/s.

FIG. 35 shows CFU-H data in a graphical presentation. The data indicates no effect of shear on aBMC samples when passed through needle at a flow-rate of 0.25 mL/s.

FIG. 36 shows viability data in a graphical presentation. The data indicates no effect of shear on aBMC samples when passed through needle at a flow-rate of 0.25 mL/s.

FIG. 37 shows CD45+ cell viability data in a graphical presentation. The data indicates no effect of shear on aBMC samples when passed through needle at a flow-rate of 0.25 mL/s.

FIG. 38 shows CD45+ cell viability data in a graphical presentation of one ANOVA statistical analysis indicating no effect of shear on aBMC samples when passed through needle at a flow-rate of 0.25 mL/s.

FIGS. 39A, 39B and 39C show cytokine levels in aBMC plasma pre-needle, post-TIN and SHN needles as assessed by ELISA. No differences were observed in aBMC plasma cytokine levels before and after passing through the needles. FIGS. 39A, 39B and 39C show the pre- and post-needle plasma levels of VEGF, SDF-1 and Endostation, respectively.

FIG. 40 shows Human Umbilical Vein Endothelial Cells (HUVEC) migration upon stimulation with plasma obtained from aBMC passed through cannula side ported needles (TIN).

FIG. 41 shows VEGF levels in aBMC cell-conditioned media. ELISA-based detection of VEGF in unconcentrated and concentrated (measured only in 5 samples—BM0008 to BM00012) conditioned media. SHN and TIN: VXP processed aBMC passed through standard hypodermic hypodermic cannula needles (SHN) or cannula side ported needles (TIN), respectively, prior to conditioning medium.

FIG. 42 shows HUVEC migration upon stimulation with conditioned medium obtained from aBMC passed through cannula side ported needles. EBM-2 with 0.1% BSA served as basal (negative) control, EGM-2 as full endothelial medium (positive) control.

FIG. 43 shows HUVEC tube formation upon stimulation with post TIN aBMC plasma. Shown are exemplary images (contrast-enhanced using Adobe Photoshop Elements, version 13). As assay input, plasma from post-TIN aBMC was diluted 1:5 with EBM-2 supplemented with 0.1% BSA. Basal medium (EBM-2 with 0.1% BSA) served as negative control, full endothelial medium (EGM-2) as positive control.

FIG. 44 shows an illustration from Documentation to Angiogenesis Analyzer for Image J visualizing angiogenesis analysis parameters.

FIGS. 45A and 45B show HUVEC tube formation upon stimulation with post-TIN aBMC plasma. As assay input, plasma from post-TIN aBMC was diluted 1:5 with EBM-2 supplemented with 0.1% BSA. Basal medium (EBM-2 with 0.1% BSA) served as negative control, full endothelial medium (EGM-2) as positive control. 3-4 images per sample were quantified with the Angiogenesis Analyzer from Image J (NIH). To minimize area selection bias, the median values were used for comparative assessments. The following Angiogenesis Analyzer output parameters are shown: “Tot. length” (A), “Nb Junctions” (B). One sample t-Tests suggest that both Total Tube Length (A) and Number of Junctions (B) are significantly higher in the aBMC sample group compared to the negative control sample (EBM-2 with 0.1% BSA).

FIGS. 46A, 46B and 46C show prevention of cell aggregation by CCM by addition of 2% post-TIN aBMC Plasma. FIG. 46A shows example images of BM0011 and BM0013 taken after 16 hours of incubation. FIG. 46B shows Total Tube length is statistically significantly higher (p<0.05) when stimulating with CCM+2% aBMC plasma compared to 2% aBMC plasma-only. FIG. 46C shows Number of Junctions is statistically significantly higher (p<0.05) when stimulating with CCM+2% aBMC plasma compared to 2% aBMC plasma-only.

FIG. 47 show improvement in rest pain in patients following treatment with the autologous bone marrow concentrate (aBMC). Rest pain is assessed through Visual Analog Scale (VAS) testing using a psychometric (self-report) response scale scored between 0 to 10 (0=No pain/hurt, 2=Hurts a little/pain is mild, 4=Hurts a little more/pain is causing discomfort, 6=Hurts even more/pain is distressing, 8=Hurts a whole lot/pain is horrible, 10=Hurts worse/pain is excruciating). The mean rest pain score at baseline was 7.67, indicating that the majority of the patients had severe pain at rest. At the post-treatment 6 month follow-up visit, all available patients showed significant improvement, where the mean rest pain score was 0.67, with four out of six patients experienced no pain at rest.

FIG. 48 show improvement in the integument for ulceration in a patient treated with autologous bone marrow concentrate (aBMC). At baseline, 5 of 6 patients were suffering from foot ulcers or open wounds. At the 6 month follow-up, none of the patients presented with ulcers or gangrene or open wounds.

### DETAILED DESCRIPTION

“Critical limb ischemia,” (CLI) as described herein, refers to an advanced stage of peripheral artery disease. It includes ischemic rest pain, arterial insufficiency ulcers, and gangrene. The latter two conditions are jointly referred to as tissue loss, reflecting the development of surface damage to the limb tissue due to the most severe stage of ischemia. CLI has a negative prognosis within a year after the initial diagnosis, with 1-year amputation rates of approximately 12% and mortality of 50% at 5 years and 70% at 10 years.

CLI can cause severe blockage in the arteries of the lower extremities, which markedly reduces blood-flow. It is a serious form of peripheral arterial disease, or PAD. PAD is caused by atherosclerosis, the hardening and narrowing of the arteries over time due to the buildup of fatty deposits called plaque. CLI can result in severe pain in the feet or toes or limbs, even while resting. Complications of poor circulation can include sores and wounds that won't heal in the legs and feet. Left untreated, the complications of CLI will result in amputation of the affected limb.

Immediate treatment to re-establish blood flow to the affected areas is necessary in order to preserve the limb. There are minimally invasive endovascular therapies which are dependent on the location as well as the severity of the blockages. Examples of treatment can include angioplasty (cutting balloon, cold balloon (CryoPlasty)) and Stents (Balloon expanded, self-expanding), laser atherostomy and directional atherectomy. In some embodiments described herein, a method of ameliorating critical limb ischemia or a condition associated with critical limb ischemia in a subject in need, is provided.

“Peripheral vascular disease,” as described herein, refers to peripheral artery disease (PAD), which is a narrowing or occlusion by atherosclerotic plaques of arteries outside of the heart and brain. The risk factors for peripheral artery disease include elevated blood cholesterol, diabetes, smoking, hypertension, inactivity, and overweight/obesity. The symptoms of peripheral artery disease depend upon the location and extent of the blocked arteries. The most common symptom of peripheral artery disease is intermittent claudication, manifested by pain (usually in the calf) that occurs while walking and dissipates at rest.

For diagnosis, upon suspicion of PAD, and/or CLI, a first study that can be performed is an ankle brachial pressure index to read the blood pressure in the ankles and the arms in order to make a comparison. When the blood pressure readings in the ankles are lower than that in the arms, blockages in the arteries which provide blood from the heart to the ankle are suspected. Normal ABI ranges from 1.00 to 1.40. The patient is diagnosed with PAD when the ABI is ≤0.90. ABI values of 0.91 to 0.99 are considered “borderline” and values >1.40 indicate non-compressible arteries. PAD is graded as mild to moderate if the ABI is between 0.41 and 0.90, and an ABI less than 0.40 is suggestive of severe PAD. These relative categories have prognostic value. If an abnormally high ABI is obtained, a lower limb Doppler ultra sound examination can be performed to look at site and extent of atherosclerosis. Other imaging can be performed by angiography, for example, where a catheter is inserted into the common femoral artery and selectively guided to the artery in question. While injecting a radiodense contrast agent an X-ray is taken. Any flow limiting stenosis found in the x-ray can be identified and treated by atherectomy, angioplasty or stenting. Contrast angiography is the most readily available and widely used imaging technique. Other techniques can include imagery such as multislice computerized tomography (CT) scanners, which provide direct imaging of the arterial system as an alternative to angiography. Another alternative is to use magnetic resonance angiography (MRA), which is a noninvasive diagnostic procedure that uses a combination of a large magnet, radio frequencies, and a computer to produce detailed images to provide pictures of blood vessels inside the body. The advantages of MRA include its safety and ability to provide high-resolution three-dimensional (3D) imaging of the entire abdomen, pelvis and lower extremities in one sitting. In some embodiments, methods for ameliorating or inhibiting critical limb ischemia or a condition associated with critical limb ischemia in a subject comprise identifying a subject having a peripheral vascular disease, peripheral arterial disease, critical limb ischemia or a condition associated with critical limb ischemia. In some embodiments, this identifying step is performed by ankle brachial pressure index (ABPI/ABI), lower limb Doppler ultrasound examination, angiography, multislice computerized tomography or by magnetic resonance angiography (MRA). In some embodiments, said methods for ameliorating or inhibiting critical limb ischemia or a condition associated with critical limb ischemia are performed on a subject that has obesity, high cholesterol, high blood pressure, and/or atherosclerosis.

“Bone marrow total nucleated cells,” as described herein, refers to a heterogeneous mixture of bone marrow-derived cells containing Hematopoietic Stem Cells (“HSCs”), Mesenchymal Stem Cells (“MSCs”), Endothelial Progenitor Cells (“EPCs”), CXCR4 positive cells, White Blood Cells (“WBCs”), and/or stromal cells. In preferred embodiments, this heterogeneous mixture of bone marrow-derived cells comprises both CD34⁺ and CD34⁻ bone marrow stem cells. In some embodiments, the stromal cells may be lineage negative/dim, CD45 negative/dim and/or CD73 positive cells.

“Stromal cells” as described herein, refers to connective tissue cells from an organ. Without being limiting, the stromal cells can come from, for example, the endometrium, bone marrow, and the ovary. Stromal cells support the function of parenchymal cells. Stromal cells can be lineage negative, in which the cells (lin−) are heterogeneous and contain a small percentage of true stem cells and a larger majority of progenitor cells. The lineage negative cells can be enriched for hematopoietic stem cells. CD34 is a type I transmembrane protein that can be present in hematopoietic cells, except erythrocytes, and can assist in cell activation. Some stromal cells are CD45 negative/dim. CD73, a protein, is also known as 5′nucleotidase or ecto-5′-nucleotidase, which is an enzyme that can convert AMP to adenosine. Some stromal cells can be CD73 positive cells. In some embodiments, methods of ameliorating or inhibiting critical limb ischemia or a condition associated with critical limb ischemia in a subject comprise identifying a subject having a peripheral vascular disease, peripheral arterial disease, critical limb ischemia or a condition associated with critical limb ischemia and providing to said identified subject a composition comprising a cell population, wherein the cell population comprises bone marrow total nucleated cells and red blood cells. In some embodiments of these methods, the cell population comprises stromal cells. In some embodiments, the stromal cells are lineage negative/dim, CD45 negative/dim and/or CD73 positive cells.

“Mesenchymal cells,” or “mesenchymal stem cells,” as described herein, refers to multipotent stromal cells that can differentiate into a variety of cell types, which can include, but are not limited to osteoblasts (bone cells), chondrocytes (cartilage cells), muscle cells and/or adipocytes (fat cells). In some embodiments of the methods provided herein, a composition comprising a cell population further comprises mesenchymal stem cells.

In some embodiments, the cell population comprises at least 1×10⁸ total nucleated cells (TNCs), the cell population is processed from 120-180 mL bone marrow aspirate and is concentrated for a final 20 mL volume as enumerated intra-operatively using a rapid diagnostic instrument to guide the aspiration of at least 120 mL but no more than 180 mL of autologous bone marrow, wherein the cells comprise a cell viability ≥70% and/or a white blood cell recovery ≥80%. In some embodiments, the cells comprise a cell viability that is, is about, is more than, is at least, 70%, 75%, 80%, 85%, 90%, 95%, 99%, or a cell viability that is within a range defined by any two of the aforementioned values. In some embodiments, the cells comprise a white blood cell recovery that is, is about, is more than, is at least, 80%, 85%, 90%, 95%, 99%, or a white blood cell recovery that is within a range defined by any two of the aforementioned values. In some embodiments, the cell population is processed from a bone marrow aspirate that is, is about, is less than, is more than, 120 mL, 130 mL, 140 mL, 150 mL, 160 mL, 170 mL or 180 mL, or any volume that is within a range defined by any two of the aforementioned values.

“Hematopoietic stem cells (HSCs),” as described herein, refers to blood cells that give rise to all the other blood cells and are derived from mesoderm. HSCs are located in red bone marrow. HSCs can give rise to many cells. Without being limiting, this can include myeloid (monocytes and macrophages, neutrophils, basophils, eosinophils, erythrocytes, megakaryocytes/platelets, dendritic cells), and lymphoid lineages (T-cells, B-cells, NK-cells).

In some embodiments, the composition provided or administered to the subject in need thereof comprises a bone marrow-derived cell population that comprises hematopoietic stem cells and red blood cells (e.g., 2%, 3%, 4%, 5%, 6%, 7%, 8%, 9%, 10%, 11%, 12%, 13%, 14%, 15%, 16%, 17%, 18%, 19%, or 20% packed total cell fraction (total cell volume of the composition) or red blood cell fraction of the composition by volume (also known as % hematocrit (% HCT) or an amount of total cells or red blood cells that is within a range defined by any two of the aforementioned values). Such packed cell fractions (total cell volume) or red blood cell fraction (% hematocrit) can be determined by microhematocrit centrifugation.

“Endothelial progenitor cells,” as described herein, refers to a population of cells that circulate in the blood, which have the ability to differentiate into endothelial cells, the cells that make up the lining of blood vessels. In some embodiments, the composition provided or administered to the subject in need thereof comprises a bone marrow-derived cell population that comprises endothelial progenitor cells.

“CXCR4,” as described herein, refers to C—X—C chemokine receptor type 4, and is also known as fusin or CD128, which is a protein, that acts as an alpha chemokine receptor that is specific for stromal derived factor 1. In some embodiments, the composition provided or administered to the subject in need thereof comprises a bone marrow-derived cell population that comprises hematopoietic stem cells, endothelial progenitor cells and CXCR4 positive cells.

“Anticoagulant” as described herein, refers to a class of drugs that work to prevent the coagulation (clotting) of blood. For example, a group of pharmaceuticals called anticoagulants can be used as an injection into human beings as a medication for thrombotic disorders. Some anticoagulants are used in medical equipment, such as test tubes, blood transfusion bags, and renal dialysis equipment. Anticoagulants reduce blood clotting which can help prevent deep vein thrombosis, pulmonary embolism, myocardial infarction and ischemic stroke.

Therapeutic uses of anticoagulants include atrial fibrillation, pulmonary embolism, deep vein thrombosis, venous thromboembolism, congestive heart failure, stroke, myocardial infarction, and genetic or acquired hypercoagulability. Examples of anticoagulants, can include, but are not limited to Alteplase, Ardeparin, Dalteparin, Danaparoid, Enoxaparin, Fondaparinux, Lepirudin, Urokinase, coumarin, vitamin K antagonist, an indirect thrombin inhibitor, heparin, a factor Xa inhibitor, a direct thrombin inhibitor, batroxobin, hemetin, a purified plant extract, EDTA, citrate, oxalate, nitrophorin and bivalirudin. In some embodiments, methods of ameliorating or inhibiting critical limb ischemia or a condition associated with critical limb ischemia in a subject comprise identifying a subject having a peripheral vascular disease, peripheral arterial disease, critical limb ischemia or a condition associated with critical limb ischemia and providing to said subject a composition comprising a cell population, comprising bone marrow total nucleated cells and red blood cells, an anticoagulant, and autologous plasma. In some embodiments, said composition comprises an anticoagulant, wherein the anticoagulant is Alteplase, Ardeparin, Dalteparin, Danaparoid, Enoxaparin, Fondaparinux, Lepirudin, Urokinase, coumarin, vitamin K antagonist, an indirect thrombin inhibitor, heparin, a factor Xa inhibitor, a direct thrombin inhibitor, batroxobin, hemetin, a purified plant extract, EDTA, citrate, oxalate, nitrophorin or bivalirudin. In some embodiments, the anticoagulant is heparin, the final heparin concentration in bone-marrow aspirate is about 100 IU/mL.

“Hypodermic needle,” as described herein, refers to a hollow needle that can be used with a syringe to inject substances into the body or extract fluids from it. They may also be used to take liquid samples from the body, for example taking blood from a vein in venipuncture. Large bore hypodermic intervention is especially useful in catastrophic blood loss or shock.

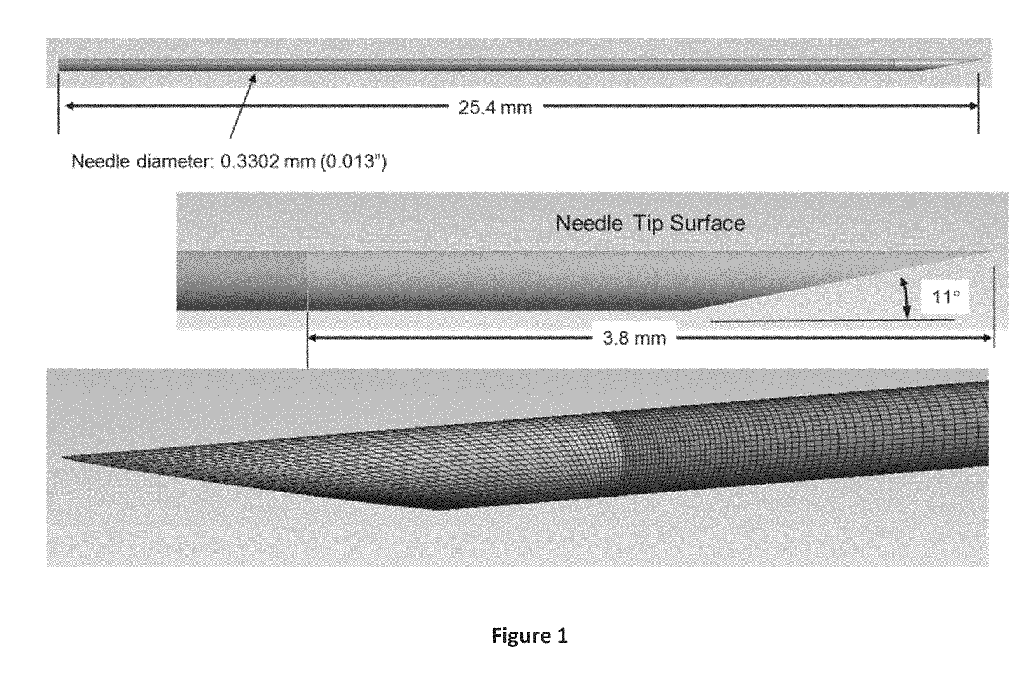

A standard “Cannula needle,” as described herein, refers to a needle or a tube that can be inserted into the body, often for the delivery or removal of fluid or for the gathering of fluid for data. In simple terms, a cannula can also surround the inner or outer surfaces of a trocar needle thus extending needle approach to a vein by half or more of the length of the introducer. As shown in FIG. 1, is an exemplary standard cannula needle.

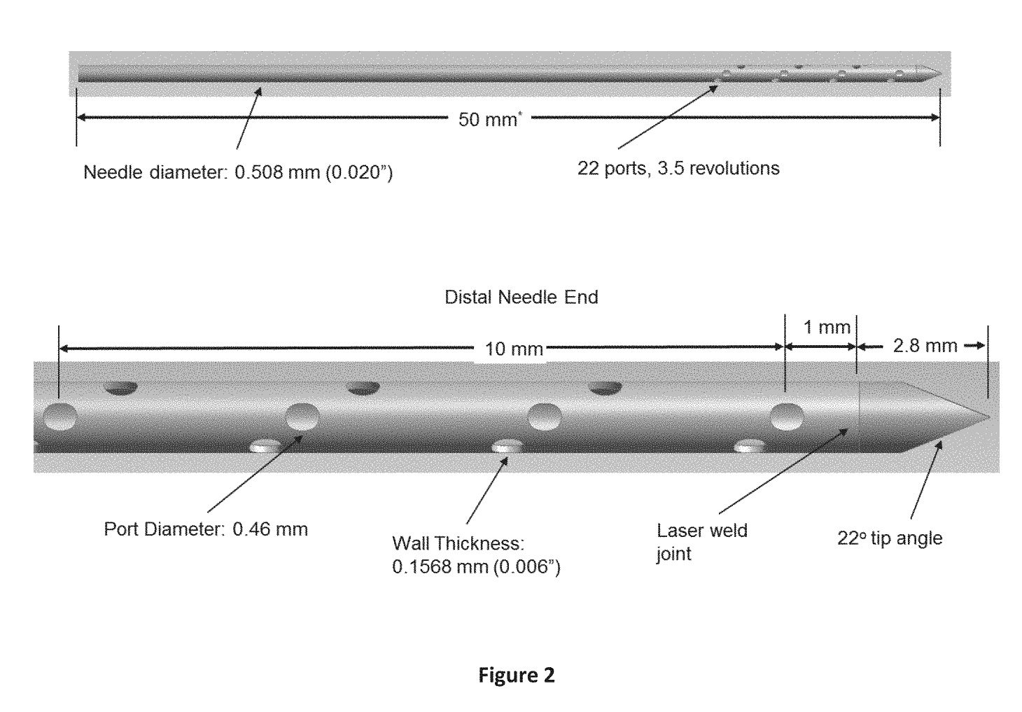

A “side ported” or side port needle, or cannula needle, as described herein, can refer to a needle that provides multiple ports along the shaft of the needle to the distal tip for a controlled delivery of a substance. As shown in FIG. 2, is an exemplary cannula side ported needle. A side port needle for the delivery of cells can have the advantage of leading to a greater distribution of cells upon administration.

A “catheter,” as described herein, refers to a thin tube that can be inserted into a body to treat disease or to perform a medical procedure. A catheter can also have multiple side ports along the length of the catheter tube.

“Viscosity,” as described herein describes a measure of a fluid and its resistance to gradual deformation by shear stress.

“Shear Stress,” is a component of stress coplanar with a material cross section. Shear stress can arise from a force vector component parallel to the cross section. Shear stress and shear induced damage to cells is an imminent problem that needs to be solved in order to efficiently deliver an effective amount of viable cells for treatment for ailments, such as for example, CLI.

In some embodiments, methods of ameliorating or inhibiting critical limb ischemia or a condition associated with critical limb ischemia in a subject comprise identifying a subject having a peripheral vascular disease, peripheral arterial disease, critical limb ischemia or a condition associated with critical limb ischemia and providing to said subject a composition comprising a cell population, wherein the cell population comprises bone marrow total nucleated cells and red blood cells (e.g., 2%, 3%, 4%, 5%, 6%, 7%, 8%, 9%, 10%, 11%, 12%, 13%, 14%, 15%, 16%, 17%, 18%, 19%, or 20% packed total cell fraction of the composition (total cell volume of the composition) or red blood cell fraction of the composition or an amount of total cells or red blood cells in the composition that is within a range defined by any two of the aforementioned values), an anticoagulant, and autologous plasma, wherein the composition has a viscosity of 1.5 to 5.0 centipoise (cP) measured at 37° C., wherein the composition comprises a viable cell dose and, wherein said composition is administered to said subject intramuscularly through a standard terminally-ported cannula needle, or a cannula side-ported needle or catheter comprising a plurality of ports and a closed end. Such packed cell fractions (total cell volume) or red blood cell fraction (% hematocrit) can be determined by microhematocrit centrifugation or other indirect methods. In some embodiments, the needle or catheter has a lumen size (diameter) of 0.33 mm. In some embodiments, the needle or catheter has a lumen size (diameter) of 0.51 mm. In some embodiments, the needle or catheter has a lumen size (diameter) of 0.69 mm. In some embodiments, the needle or catheter comprises 10-24 side ports. In some embodiments, the side ports have a diameter of 0.46 mm, 0.48 mm, 0.50 mm, 0.52 mm. 0.54 mm, or 0.56 mm, or a diameter within a range defined by any two of the aforementioned values. In some embodiments, the composition is administered at a flow rate that is, is about, is less than, 0.1 ml/s, 0.2 ml/s, 0.3 ml/s, 0.4 ml/s, 0.5 ml/s, or a rate within a range defined by any two of the aforementioned rates. In some embodiments, the composition is administered at a rate of 0.25 mL per second. In some embodiments, by utilization of the methods described herein, the administered composition has a muscle distribution area, representative of a volume distribution using a two-dimensional image, that is, is about, is at least, 3 cm², 4 cm², 5 cm², 6 cm², 7 cm², 8 cm², 9 cm² or 10 cm², or a distribution area that is within a range defined by any two of the aforementioned values. In some embodiments, the composition is administered at a pressure that is, is about, is less than, 1, 2, 3, 4, 5, 6, 7, 8, 9, or 10 psi or within a range defined by any two of the aforementioned pressures. In some embodiments, said compositions are administered to said subjects intramuscularly at a shear stress of less than or equal to 400 Pa. In some embodiments, the shear stress upon delivery of said compositions is, is about, is less than, 25 Pa, 50 Pa, 75 Pa, 100 Pa, 125 Pa, 150 Pa, 175 Pa, 200 Pa, 225 Pa, 250 Pa, 275 Pa, 300 Pa, 325 Pa, 350 Pa, 375 Pa, or 400 Pa, or a shear stress within a range defined by any two of the aforementioned values. In some embodiments, the methods further comprise measuring in said subject an increased blood flow, an increase in perfusion, an increase in transcutaneous oxygen, an increase in angiogenesis, an increase in vascularity, or an increase in amputation-free survival rate after administration of said composition.

“Viable cell dose,” as described herein, refers to the measure of the number of cells that are viable, for example, alive and capable of growth, in a given area and/or volume. Cell viability may be assessed by a variety of assays, such as assays for exclusion of Propidium iodide, trypan blue, or 7-Aminoactinomycin D. The viable cell count can be determined, for example by the total cell count subtracting out the count of nonviable or dead cells. In some embodiments, the viable cell dose given to the subject, as measured after the bone marrow aspiration and after the bedside processing but prior to injection at the point of care, is within a range of 1.75×10⁷ to 7.67×10⁷ white blood cells per mL. In some embodiments, the viable cell dose is, is about, is less than, is more than, 1.75×10⁷ white blood cells per mL, 2.0×10⁷ white blood cells per mL, 3.0×10⁷ white blood cells per mL, 4.0×10⁷ white blood cells per mL, 5.0×10⁷ white blood cells per mL, 6.0×10⁷ white blood cells per mL, 7.0×10⁷ white blood cells per mL or 7.67×10⁷ white blood cells per ml, or an amount within a range defined by any two of the aforementioned amounts. In some embodiments, the viable cell dose delivered to the subject in need thereof is 4.0×10⁷ white blood cells per ml.

In some embodiments, the viable cell dose given to the subject in need thereof, as measured after the bedside processing, is, is about, is less than, is more than, 3.65×10⁶ mononuclear cells per mL, 4.0×10⁶ mononuclear cells per mL, 5.0×10⁶ mononuclear cells per mL, 6.0×10⁶ mononuclear cells per mL, 7.0×10⁶ mononuclear cells per mL, 8.0×10⁶ mononuclear cells per mL, 9.0×10⁶ mononuclear cells per mL, 1.0×10⁷ mononuclear cells per mL, 2.0×10⁷ mononuclear cells per mL, or 2.15×10⁷ mononuclear cells per mL or an amount within a range defined by any two of the aforementioned amounts. In some embodiments, the viable cell dose given to the subject, as measured after the bedside processing, is 1.08×10⁷ mononuclear cells per mL.

In some embodiments, the viable cell dose given to the subject, as measured after the bedside processing, is, is about, is less than, is more than, 5×10³ colony forming units (CFU-H) per mL, 6.0×10³ colony forming units (CFU-H) per mL, 7.0×10³ colony forming units (CFU-H) per mL, 8.0×10³ colony forming units (CFU-H) per mL, 9.0×10³ colony forming units (CFU-H) per mL, 1.0×10⁴ colony forming units (CFU-H) per mL, 2.0×10⁴ colony forming units (CFU-H) per mL, 3.0 1.0×10⁴ colony forming units (CFU-H) per mL, 4.0×10⁴ colony forming units (CFU-H) per mL, 5.0×10⁴ colony forming units (CFU-H) per mL, 6.0×10⁴ colony forming units (CFU-H) per mL, 7.0×10⁴ colony forming units (CFU-H) per mL, 8.0×10⁴ colony forming units (CFU-H) per mL, 9.0×10⁴ colony forming units (CFU-H) per mL, 1.0×10⁵ colony forming units (CFU-H) per mL, or 2.0×10⁵ colony forming units (CFU-H) per mL or an amount within a range defined by any two of the aforementioned amounts. In some embodiments, the viable cell dose given to the subject, as measured after the bedside processing, is 5.0×10³ colony forming units (CFU-H) per mL.

“Hematocrit (HCT),” as described herein, refers to a packed total cell volume of the composition or the erythrocyte volume fraction (EVF) of the composition. Such packed cell fractions (total cell volume) or red blood cell fraction (% hematocrit) can be determined by microhematocrit centrifugation or the hematocrit can be calculated by an automated analyzer such that it is not directly measured. By an automated process, for example, HCT is determined by multiplying the red cell count by the mean cell volume. The hematocrit can be slightly more accurate as the packed cell volume (PCV) may include small amounts of blood plasma trapped between the red cells. An estimated hematocrit as a percentage may be derived by tripling the hemoglobin concentration in g/dL and dropping the units.

The packed cell volume (PCV) can be determined, for example, by centrifuging heparinized blood/bone marrow composition in a capillary tube (microhematocrit tube) at 10,000 RPM for five minutes to ten minutes. This can allow for the separation of the blood/bone marrow composition into layers. The volume of packed red blood cells divided by the total volume of the blood/bone marrow composition gives the PCV. Since a tube is used, this can be calculated by measuring the lengths of the layers. In some embodiments, packed total cell fraction (total cell volume) can be determined that includes all cell types, including red blood cells and nucleated cells in a blood/bone marrow composition.

Another way of measuring hematocrit levels can be performed, for example, through optical methods such as spectrophotometry. Through differential spectrophotometry, the differences in optical densities of a blood sample/bone marrow composition flowing through small-bore glass tubes at isobestic wavelengths for deoxyhemoglobin and oxyhemoglobin and the product of the luminal diameter and hematocrit create a linear relationship that is used to measure hematocrit levels is the volume percentage of red blood cells in the blood/bone marrow composition. In some embodiments, the hematocrit levels are determined through differential spectrophotometry.

In some embodiments, methods of ameliorating or inhibiting critical limb ischemia or a condition associated with critical limb ischemia in a subject comprise identifying a subject having a peripheral vascular disease, peripheral arterial disease, critical limb ischemia or a condition associated with critical limb ischemia and providing to said subject a composition comprising a cell population that comprises bone marrow total nucleated cells and red blood cells (e.g., 2%, 3%, 4%, 5%, 6%, 7%, 8%, 9%, 10%, 11%, 12%, 13%, 14%, 15%, 16%, 17%, 18%, 19%, or 20% packed total cell fraction of the composition (total cell volume of the composition) or red blood cell fraction of the composition or an amount of total cells or red blood cells that is within a range defined by any two of the aforementioned values), an anticoagulant, and autologous plasma, wherein the composition has a viscosity of 1.5 to 5.0 centipoise (cP) measured at 37° C., wherein the composition comprises a viable cell dose and, wherein said composition is administered to said subject intramuscularly through a standard terminally-ported cannula needle, or a cannula side-ported needle or catheter comprising a plurality of ports and a closed end. A preferred viscosity of said compositions is 1.6 cP-5.0 cP, and a most preferred viscosity is 1.8 cP-3.0 cP, and a most preferred viscosity is 2.0 cP to 2.5 cP. In some embodiments, the viscosity of said compositions is, is about, is less than, 1.5 cP, 1.6 cP, 1.7 cP, 1.8 cP, 1.9 cP, 2.0 cP, 2.1 cP, 2.2 cP, 2.3 cP, 2.4 cP, 2.5 cP, 2.6 cP, 2.7 2.8 cP, 2.9 cP, 3.0 cP, 3.1 cP, 3.2 cP, 3.3 cP, 3.4 cP, 3.5 cP, 3.6 cP, 3.7 cP, 3.8 cP, 3.9 cP, 4.0 cP, 4.1 cP, 4.2 cP, 4.3 cP, 4.4 cP, 4.5 cP, 4.6 cP, 4.7 cP, 4.8 cP, 4.9 cP, or 5.0 cP, or a viscosity that is within a range defined by any two of the aforementioned viscosities. In some embodiments, said cell population comprises a packed total cell fraction of the composition (total cell volume of the composition) or red blood cell fraction of the composition that is, is about, is less than, is more than, 2%, 3%, 4%, 5%, 6%, 7%, 8%, 9%, 10%, 11%, 12%, 13%, 14%, 15%, 16%, 17%, 18%, 19%, or 20%, or any percentage of packed total cell fraction of the composition or red blood cell fraction of the composition between any two values defined herein (also known as % hematocrit (% HCT)). In some embodiments, the said cell population comprises 2-20% packed total cell fraction of the composition (total cell volume of the composition) or red blood cell fraction of the composition (also known as % hematocrit (% HCT)).

“Cell recovery” refers to the percent of cells in a sample that can be recovered after undergoing the concentration process on the basis of the total number of cells present in the initial and final sample. In some embodiments of the methods provided herein, the aspirated bone marrow has a white blood cell recovery ≥80%.

In some embodiments, the composition comprises bone marrow total nucleated cells and red blood cells (e.g., 2%, 3%, 4%, 5%, 6%, 7%, 8%, 9%, 10%, 11%, 12%, 13%, 14%, 15%, 16%, 17%, 18%, 19%, or 20% packed total cell fraction of the composition (total cell volume of the composition) or red blood cell fraction of the composition or an amount of total cells or red blood cells that is within a range defined by any two of the aforementioned values), and CD34⁺ and CD34⁻ bone marrow stem cells. CD34 is a protein that resides on the cell surface that can function in cell-cell adhesion. It can also mediate the attachment of stem cells to bone marrow extracellular matrix or directly to stromal cells. The levels of CD34 are important as it is correlated with improved survival after donor bone marrow transplantations and can play a role in the outcomes such as survival and graft versus host disease.

“Perfusion,” as described herein, refers to a process in which the body delivers blood to a capillary bed in its biological tissue. Tests can be performed to verify that adequate and increased perfusion exists during a patient's assessment process that is performed by medical or emergency personnel. The most common methods include evaluating a body's skin color, temperature, condition and capillary refill. Tests can also be performed to verify that adequate and increased skin perfusion exists during a patient's assessment process that is performed by medical or emergency personnel. The most common methods include evaluating transcutaneous oxygen pressure (TcPO2) or skin perfusion pressure (SPP). Measuring perfusion can be performed by many techniques, without being limiting, examples can include magnetic resonance imaging (MRI), and CT (contrast enhanced tomography).

“Angiogenesis,” as described herein, refers to the physiological process through which new blood vessels form from pre-existing vessels.

“Vascularity” as described herein, refers to having many circulatory vessels. In some embodiments, methods of ameliorating critical limb ischemia or a condition associated with critical limb ischemia in a subject comprise identifying a subject having a peripheral vascular disease, peripheral arterial disease, critical limb ischemia or a condition associated with critical limb ischemia and providing to said subject a composition comprising a cell population, wherein the cell population comprises bone marrow total nucleated cells and red blood cells (e.g., 2%, 3%, 4%, 5%, 6%, 7%, 8%, 9%, 10%, 11%, 12%, 13%, 14%, 15%, 16%, 17%, 18%, 19%, or 20% packed total cell fraction of the composition (total cell volume of the composition) or red blood cell fraction of the composition or an amount of total cells or red blood cells that is within a range defined by any two of the aforementioned values), an anticoagulant, and autologous plasma, wherein the composition has a viscosity of 1.5 to 5.0 centipoise (cP) measured at 37° C., wherein the composition comprises a viable cell dose and, wherein said composition is administered to said subject intramuscularly through a standard terminally-ported cannula needle, or a cannula side-ported needle or catheter comprising a plurality of ports and a closed end.

### Methods of Treating Critical Limb Ischemia

Cells for administration to subjects must be handled efficiently and carefully during delivery due to conditions that can cause damage to the cells. Cells can be exposed to tangential mechanical forces, called shear stresses, for example, which lead to cell death and the release of inflammatory molecules from the surviving cells in the cell population being delivered. Shear stress occurs because of many factors, such as the cell proximity to the lumen wall, the velocity of the injection, the radius of the lumen, and the viscosity of the fluid. These shear forces can cause damage ranging from slight morphological alterations to cell lysis. Accordingly, the reduction of shear stress damage to the cells is an important aspect to consider when delivering the compositions described herein to subjects in need thereof.

Some embodiments disclosed herein provide methods of ameliorating or inhibiting critical limb ischemia or a condition associated with critical limb ischemia in a subject, wherein said methods relate to the administering of a composition comprising a bone marrow-derived cell population.

In some embodiments, said cell population comprises bone marrow total nucleated cells and red blood cells (e.g., 2%, 3%, 4%, 5%, 6%, 7%, 8%, 9%, 10%, 11%, 12%, 13%, 14%, 15%, 16%, 17%, 18%, 19%, or 20% packed total cell fraction of the composition (total cell volume of the composition) or red blood cell fraction of the composition or an amount of total cells or red blood cells that is within a range defined by any two of the aforementioned values). In some embodiments, red blood cells may be used to adjust the viscosity of the composition.

The bone marrow-derived cell population can comprise, but not limited to, Hematopoietic stem cells (HSC) and/or Hematopoietic progenitor cells (HPC) and/or CD34 positive cells, identified following ISHAGE guidelines for CD34 positive cell enumeration; Mesenchymal stem cells (MSC) and/or stromal cells, identified as at least lineage negative/dim, CD45 negative/dim and CD73 positive cells; Endothelial progenitor cells (EPC), identified as at least CD45 negative/dim, CD34 positive and vascular endothelial growth factor receptor 2 (VEGFR2) positive cells; CXCR4 progenitor cells identified as at least lineage negative/dim, CD45 negative/dim, and CD184 (CXCR4) positive cells. The said mixture of progenitor cells should also possess colony forming capacity as assessed by colony forming unit assays for HSC/HPCs, MSCs/stromal cells and EPCs. The aforementioned cell population comprises a variety of cell types that work synergistically in the overall healing of the injured tissue.

In some embodiments, the cell population comprises CD34⁺ and CD34⁻ bone marrow stem cells. In some embodiments, the cell population comprises stromal cells. In some embodiments, the stromal cells are lineage negative/dim, CD45 negative/dim and CD73 positive cells. In some embodiments, the cell population comprises mesenchymal stem cells. In some embodiments, the cell population comprises hematopoietic stem cells, endothelial progenitor cells and CXCR4 positive cells.

In some embodiments, the bone marrow may be stratified using a device such as a disposable centrifugal cell stratification device, which may have controlled valving capable of responding to motor driven on and off positioning and may have a density functioning separation device such that bone marrow derived cell populations above or below the device may be harvested, although variations on each parameter of a device consistent with the parameters mentioned above, and alternatives comprising the use of alternative devices or for which cells are stratified without the use of a device as disclosed herein are also contemplated. Some embodiments comprise an optional on-board firmware for controlling the interrogation and harvest of certain cell populations within a multi-stratified zone driven by differences in a light source transmission value responding to pre-determined light units or an equivalency density separation device whose density equals the targeted cells density to mass ratio, a rapid analytical instrument verifying certain cell populations or cell dose and viscosity of certain populations.

In some embodiments, the bone marrow-derived cell population can be processed from about 100 mL, 110 mL, 120 mL, 130 mL, 140 mL, 150 mL, 160 mL, 170 mL, 180 mL, 190 mL, 200 mL, or more autologous bone marrow aspirate. In some embodiments, the bone marrow aspirate can be concentrated for a final volume that is, is about, is less than, is more than, 5 mL, 10 mL, 15 mL, 20 mL, 25 mL, 30 mL, 35 mL, 40 mL, 4 5 mL, 50 mL, or a range between any two of the above values. In some embodiments, the bone marrow-derived cell population can have a cell viability that is at least 10%, 20%, 30%, 40%, 50%, 60%, 70%, 80%, 90%, 95%, 98%, 99%, or a range that is between any two of the above values. In some embodiments, the bone marrow-derived cell population can have a white blood cell recovery that is at least 10%, 20%, 30%, 40%, 50%, 60%, 70%, 80%, 90%, 95%, 98%, 99%, or a range that is between any two of the above values.

In some embodiments, the composition comprises autologous plasma. In some embodiments, autologous plasma may be used to adjust the viscosity of the composition.

In some embodiments, the composition has a viscosity of 1.5 to 5.0 centipoise (cP) measured at 37° C. A viscometer, possibly including but not required ancillary software, may be used to determine the composition viscosity in units (for example, centipoise (cP)), and to calculate the required plasma volume required to yield a final composition in a preferred range of 1.8-5 cP at 98.6° Fahrenheit or 37° Celsius. In some embodiments, the composition viscosity is less than or equal to or any number in between 1.8 cP, 1.9 cP, 2.0 cP, 2.1 cP, 2.2 cP, 2.3 cP, 2.4 cP, 2.5 cP, 2.6 cP, 2.7 2.8 cP, 2.9 cP, 3.0 cP, 3.1 cP, 3.2 cP, 3.3 cP, 3.4 cP, 3.5 cP, 3.6 cP, 3.7 cP, 3.8 cP, 3.9 cP, 4.0 cP, 4.1 cP, 4.2 cP, 4.3 cP, 4.4 cP, 4.5 cP, 4.6 cP, 4.7 cP, 4.8 cP, 4.9 cP, or 5.0 cP.

In some embodiments, the composition comprises a viable cell dose. In some embodiments, the viable cell dose given to the subject as measured after the bone marrow aspiration and after the bedside processing but prior to injection at the point of care is 4.0×10⁷ white blood cells per ml. In some embodiments, the viable cell dose is 1.75×10⁷, 2.0×10⁷, 3.0×10⁷, 4.0×10⁷, 5.0×10⁷, 6.0×10⁷, 7.0×10⁷ or 7.6⁷×10⁷ white blood cells per ml, or an amount within a range defined by any two of the aforementioned amounts. In some embodiments, the viable cell does is 4.0×10⁷ white blood cells per ml. In some embodiments, the viable cell dose given to the subject and as measured after the bedside processing is 3.65×10⁶, 4.0×10⁶, 5.0×10⁶, 6.0×10⁶, 7.0×10⁶, 8.0×10⁶, 9.0×10⁶, 1.0×10⁷, 2.0×10⁷, or 2.15×10⁷ mononuclear cells per mL or an amount within a range defined by any two of the aforementioned amounts. In some embodiments, the viable cell dose given to the subject and as measured after the bedside processing is 1.08×10⁷ mononuclear cells per mL. In some embodiments, the viable cell dose given to the subject and as measured after the bedside processing is 5×10³, 6.0×10³, 7.0×10³, 8.0×10³, 9.0×10³, 1.0×10⁴, 2.0×10⁴, 3.0 1.0×10⁴, 4.0×10⁴, 5.0×10⁴, 6.0×10⁴, 7.0×10⁴, 8.0×10⁴, 9.0×10⁴, 1.0×10⁵, 2.0×10⁵ colony forming units (CFU-H) per mL or an amount within a range defined by any two of the aforementioned amounts. In some embodiments, the viable cell dose given to the subject and as measured after the bedside processing is 1.08×10⁷ (Range 3.65×10⁶ to 2.15×10⁷) mononuclear cells per mL. In some embodiments, the viable cell dose given to the subject and as measured after the bedside processing is 5.0×10³ (Range 5.0×10³ to 200×10³) colony forming units (CFU-H) per mL.

In some embodiments, the methods further comprise measuring in said subject an improvement in index limb wound healing and/or closure and/or both.

In some embodiments, the composition comprises an anticoagulant. A number of anticoagulant sources are contemplated, for example a coumarin, a vitamin K antagonist, an indirect thrombin inhibitor, heparin, a factor Xa inhibitor, a direct thrombin inhibitor, batroxobin, hemetin, a purified plant extract, EDTA, citrate, oxalate, and a nitrophorin. A preferred anticoagulant is bivalirudin.

Bivalirudin has a number of beneficial properties, such as a short (20 minute) half-life and a mode of action which is independent of many other anticoagulants, such as heparin, which may be independently administered to the patient pursuant to surgical intervention through which stem cell sources such as bone marrow sources are obtained. In some embodiments the concentration of anticoagulant, such as bivalirudin, to be used is 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, or 55 mg/mL. In some embodiments the concentration of anticoagulant, such as bivalirudin, to be used falls in the range of 1 mg/mL to 50 mg/mL, preferably 5 mg/mL to 35 mg/mL, more preferably 10 mg/mL to 25 mg/mL, most preferably 20 mg/mL. Illustratively, each vial may contain 250 mg of bivalirudin, to which 5 mL of sterile water is added for injection. One may gently swirl the anticoagulant such as bivalirudin with the water, for example until all material is dissolved and the solution appears clear. One may then aspirate the solution into a sterile syringe such as a 20 mL sterile syringe. One may then fill the syringe with 0.9% Sterile sodium chloride, for example, for injection up until 12.5 mL mark, to yield a final concentration of 20 mg/mL. One may then take three sterile 20 mL syringes and fill 2 mL of 20 mg/mL bivalirudin solution. One may then aspirate 18 mL of stem cell source in each syringe to make a final volume of 20 mL. The final concentration of the anticoagulant, such as bivalirudin, along with aspirated bone marrow, in each syringe may be 2 mg/mL. It is known to those skilled in the art of aspiration of bone marrow that the aspiration needle used is 7, 8, 9, 10, 11, 12, 13, 14, or 15 gauges, such as in the range of 8 to 14 gauges, such as such as in the range of 9 to 12 gauges, preferably the needle/trocar is of 11 gauges. The stem and/or progenitor cell source, such as bone marrow is aspirated in the syringe or syringes pre-filled with anticoagulant solution, for example for a final cumulative volume between 120 mL to 180 mL. The aspiration syringe used may have a volume of 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL, 42 mL, 43 mL, 44 mL, 45 mL, 46 mL, 47 mL, 48 mL, 49 mL, 50 mL, 51 mL, 52 mL, 53 mL, 54 mL, 55 mL, 56 mL, 57 mL, 58 mL, 59 mL, 60 mL, 61 mL, 62 mL, 63 mL, 64 mL, 65 mL, 66 mL, 67 mL, 68 mL, 69 mL, 70 mL, 71 mL, 72 mL, 73 mL, 74 mL, 75 mL, 76 mL, 77 mL, 78 mL, 79 mL, 80 mL, 81 mL, 82 mL, 83 mL, 84 mL, 85 mL, 86 mL, 87 mL, 88 mL, 89 mL, 90 mL, 91 mL, 92 mL, 93 mL, 94 mL, 95 mL, 96 mL, 97 mL, 98 mL, 99 mL, 100 mL, 101 mL, 102 mL, 103 mL, 104 mL, 105 mL, 106 mL, 107 mL, 108 mL, 109 mL, or 110 mL, preferably may fall in the range of 5 mL to 100 mL, preferably 10 to 60 mL, more preferably 20 to 50 mL, most preferably 20 mL.

In some embodiments, said composition is administered to said subject intramuscularly or intradermally through a standard terminally-ported cannula needle, or a cannula side-ported needle or catheter comprising a plurality of ports and a closed end. In some embodiments, said composition is aspirated, concentrated, and administered to said subject intramuscularly or intradermally using a standard terminally-ported cannula needle, cannula side-ported needle or catheter comprising a plurality of ports and a closed end. In some embodiments, the composition can be administered at more than 1, for example, 2, 3, 4, 5 or more sites of the subject. In some embodiments, the cannula side-ported needle or catheter has a lumen size (diameter) of 0.33 mm. In some embodiments, the cannula side-ported needle or catheter has a lumen size (diameter) of 0.51 mm. In some embodiments, the cannula side-ported needle or catheter has a lumen size (diameter) of 0.69 mm. In some embodiments, said cannula side-ported needle or catheter comprises 10-24 side ports. In some embodiments, said cannula side-ported needle or catheter comprises 10, 12, 14, 16, 18, 20 or 24 side ports. In some embodiments, aid side ports have a diameter of about 0.46 mm to about 0.56 mm.

Aspects of the method also include rates of administering the composition. Accordingly, the composition is provided to the subject at a rate of 0.1 mL to 0.5 mL per second. In some embodiments, said composition is administered to said subject at 0.25 mL to 0.5 mL per dose. In some embodiments, the composition is administered to the subject at a rate of 0.1 ml per second, 0.2 ml per second, 0.3 ml per second, 0.4 ml per second, 0.5 ml per second, or at a rate within a range defined by any two of the aforementioned values. In some embodiments, said composition is administered at a pressure that is, is about, is less than, 1 psi, 2 psi, 3 psi, 4 psi, 5 psi, 6 psi, 7 psi, 8 psi, 9 psi, 10 psi, or a range between any two of the above values. In some embodiments, said composition is administered to said subject intramuscularly at a shear stress that is, is about, is less than, 100 Pa, 200 Pa, 300 Pa, 400 Pa, 500 Pa, 600 Pa, 700 Pa, 800 Pa, 900 Pa, 1,000 Pa, or a range between any two of the above values. In some embodiments, the administered composition has a muscle distribution area, representative of a volume distribution using a two-dimensional image, that is, is about, is greater than, 1 cm², 2 cm², 3 cm², 4 cm², 5 cm², 6 cm², 7 cm², 8 cm², 9 cm², 10 cm², 11 cm², 12 cm², 13 cm², 14 cm², 15 cm², 16 cm², 17 cm², 18 cm², 19 cm², 20 cm², or a range between any two of the above values.

In some embodiments, said composition is administered in a single dose. In some embodiments, said composition is administered in at least 30 doses. In some embodiments, said composition is administered in at least 40 doses. In some embodiments, said composition is administered in a single dose per day on each of multiple days. In some embodiments, said composition is administered in at least two doses per day on each of multiple days.

In some embodiments, the methods further comprise measuring in said subject an increased blood flow, an increase in angiogenesis, an increase in vascularity, an increase in perfusion, an increase in transcutaneous oxygen, an improvement in wound healing, an increase in limb salvage, or an increase in amputation-free survival rate after administration of said composition. In some embodiments, the methods further comprise measuring in said subject an increase in amputation-free survival rate after administration of said composition.

### EXAMPLES

### Example 1. Identification of Flow Field Characteristics Based on Design

The flow field characteristics were determined based on mass flow rates, velocity and wall shear stress of a standard cannula needle and a cannula side ported needle. (FIGS. 1 and 2). Taken into consideration were the properties of the needle, such as the length, diameter, wall thickness and port diameter as shown in Table 1 below and as shown in FIGS. 1 and 2. In Table 1, the gauges (Diameter GA) of the needles refer to their outside diameter. The second dimension in parentheses, is the inside diameter/lumen size through which the fluid flows.

TABLE 1
Properties of needles.

| | | | Wall | Port |
|---|---|---|---|---|
| | Length | Diameter | Thickness | Diameter |
| Model | (cm) | GA | (in) | (mm) |
| TIN-21-5.0 | 5.0 | 19 | 0.006 | 0.46 |
| | | (0.020″) | | |
| TIN-19-7.5 | 7.5 | 21 | 0.008 | 0.56 |
| | | (0.027″) | | |
| TIN-19-10.0 | 10.0 | 21 | 0.008 | 0.56 |
| | | (0.027″) | | |
| Straight | 2.54 | 23 | NA | NA |
| Needle | | (0.013″) | | |

The standard cannula needle and side ported cannula needle exhibit different properties. As shown, needles used for testing the properties are the TIN-21-5.0, TIN-19-7.5, TIN-19-10.0, and a straight needle (standard cannula needle with no side ports). The properties of the needles were computationally tested using a non-Newtonian mixed material (10% hematocrit blood model, 90% Carreau Yasuda blood model, with an average viscosity of 3.5 cP) using a program called ANSYS CFD in which the flow of the material was assessed at a flow rate of 0.25 ml/sec (Q1) and 0.50 ml/sec (Q2).

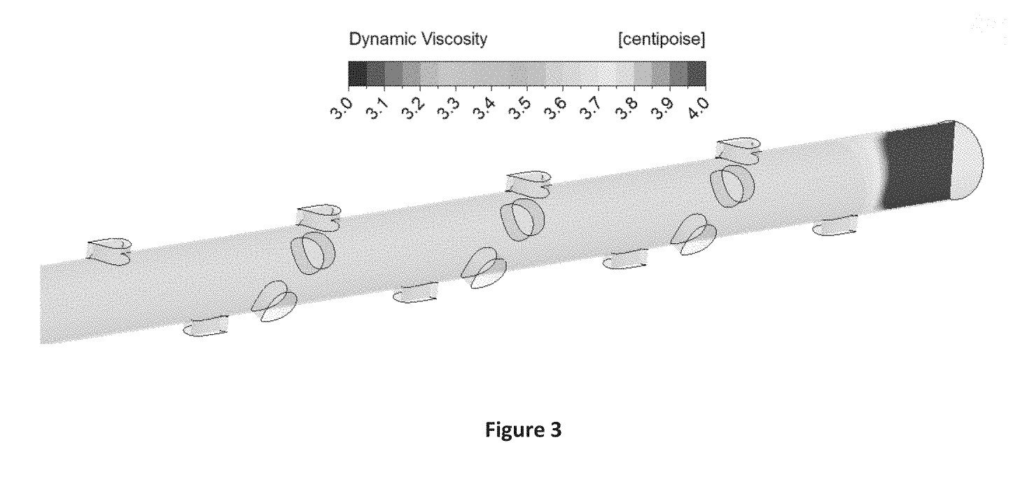

As shown, in FIG. 3, are the working fluid properties using a cannula side ported needle at steady state with a Non-Newtonian mixed material model (10% hematocrit blood model, 90% Carreau Yasuda blood model, with an average viscosity of 3.5 cP). The picture shows typical viscosity values through the needle cut planes.

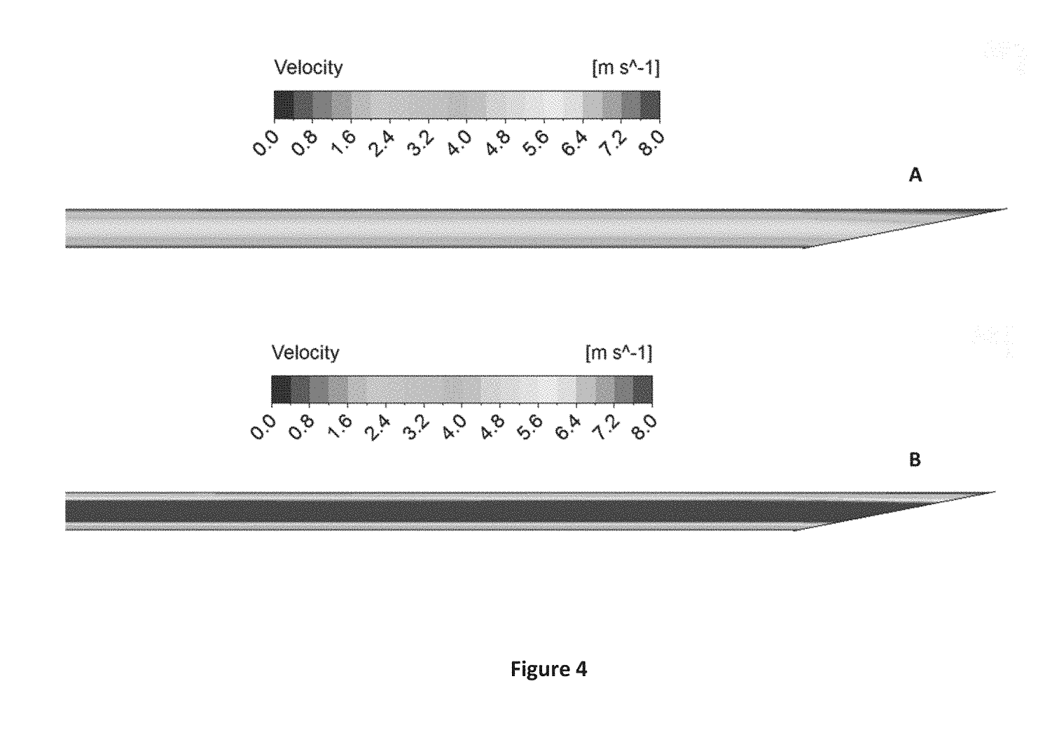

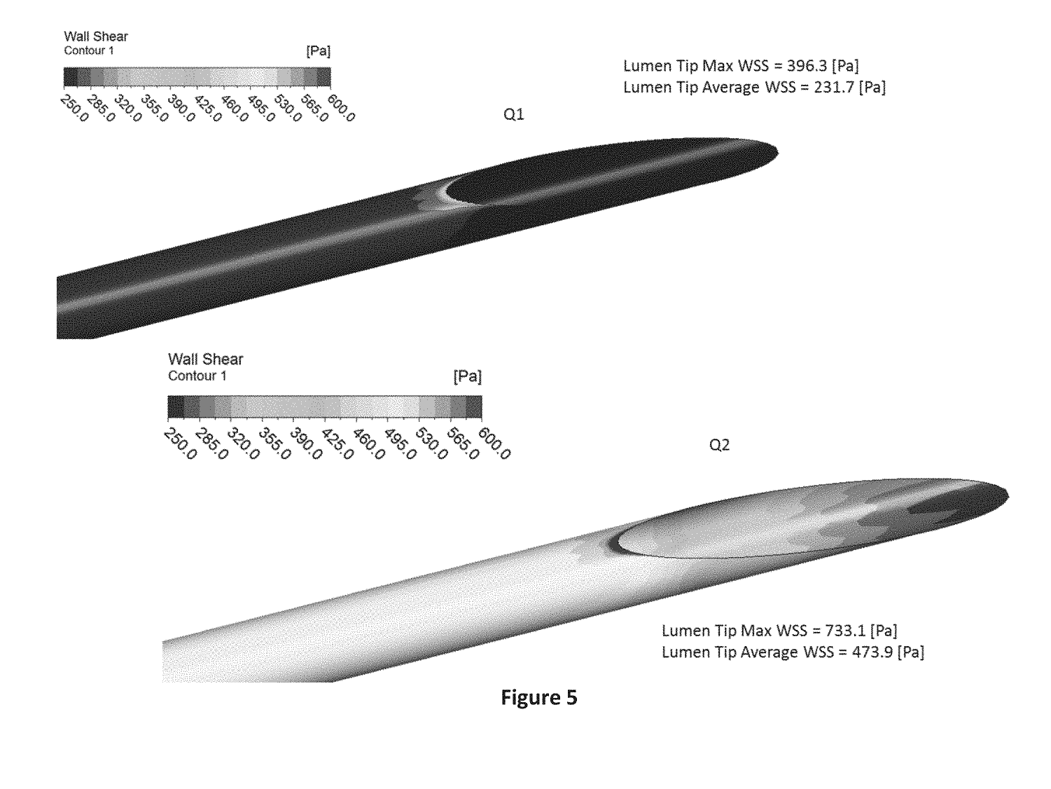

As shown in FIGS. 4A and 4B, is the velocity magnitude in a standard cannula needle at Q1 (0.25 ml/sec) and at Q2 (0.5 ml/sec), respectively. As indicated, an increase in pressure upon the fluid is exhibited at an increased flow rate which in turn can lead to damage of cells due to shear stress. As shown in FIG. 5, the wall shear stress increases with the flow rate. At Q2 (0.5 ml/sec) the fluid experience higher shear stress as compared to the low flow rate of Q1 (0.25 ml/sec). A low shear or in related terms a low flow rate can be beneficial to avoid damage to the cells for transfer.

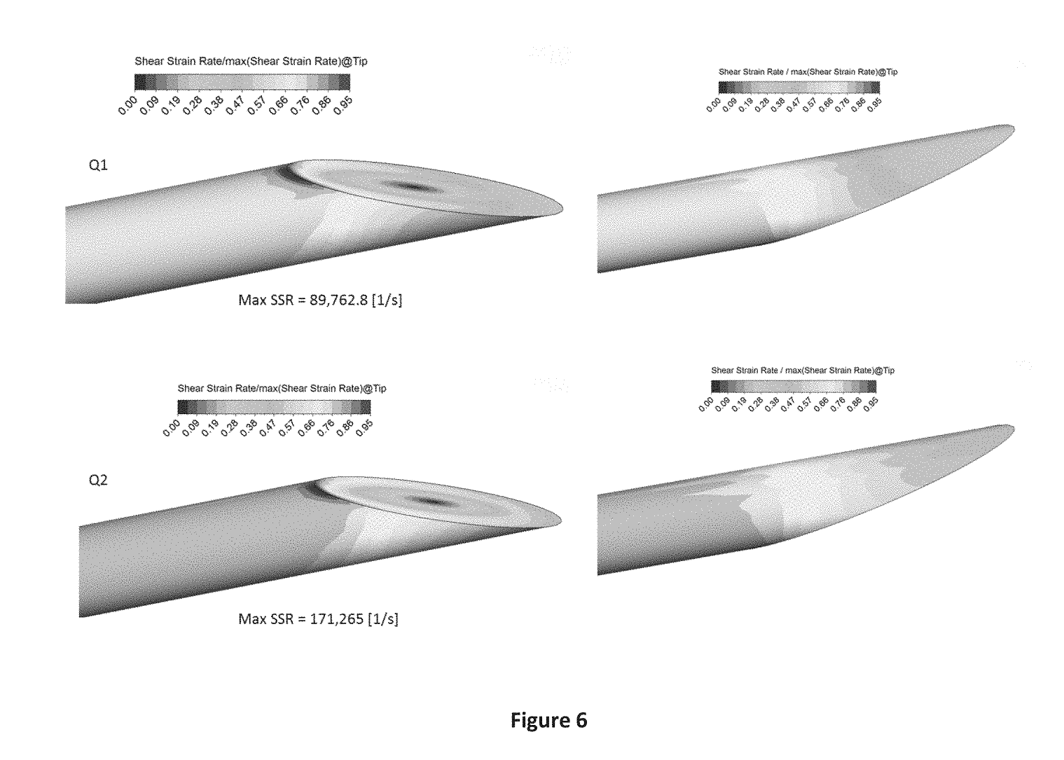

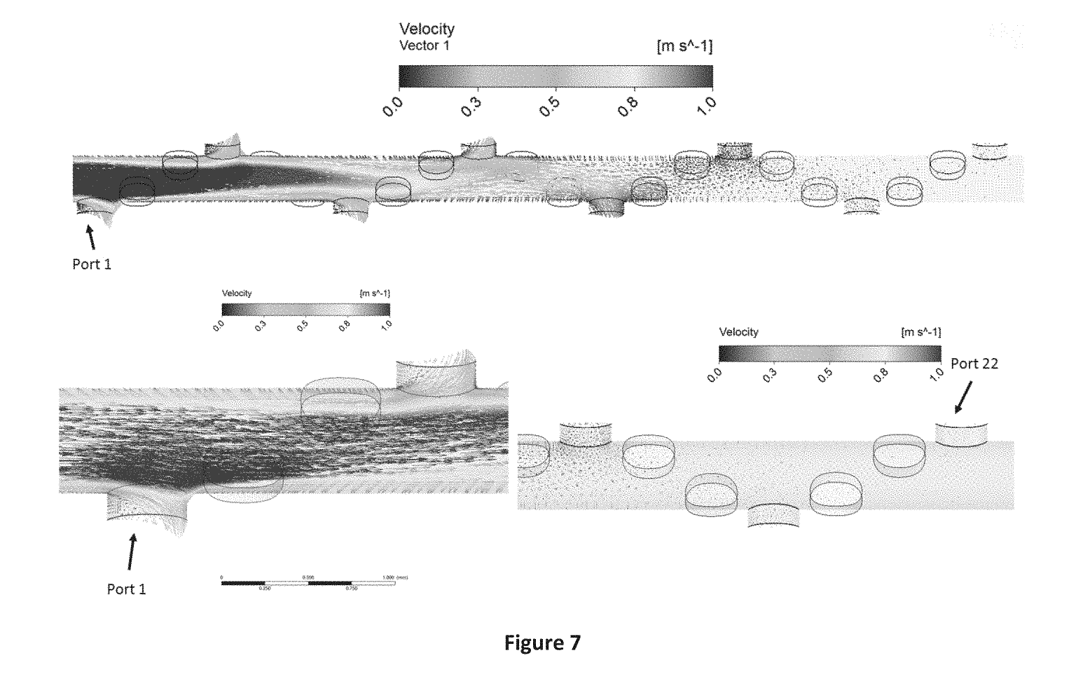

Shear strain rate refers to the change in strain (deformation) of material with respect to time. As shown in FIG. 6, is the shear strain rate using a standard cannula needle carrying a Non-Newtonian mixed material model at two rates of Q1 (0.25 ml/sec) and Q2 (0.5 ml/sec) in which the cross section of the tip of the needle is shown. As seen, the shear strain rate (1/s) is increased with the rate of fluid administration.

### Example 2. Fluid Flow Rates and their Effects when Using a Side Ported Needle Models

A computational assessment of cannula side ported needles was carried out using ANSYS CFD. For the testing fluid, a non-Newtonian mixed material (10% hematocrit blood model, 90% Carreau Yasuda blood model, with an average viscosity of 3.5 cP) was used for the sample and the needle model used was TIN-21-5.0 at a flow rate of 0.25 ml/sec (Q1). The properties of side ported needle model TIN-21-5.0 are shown in Table 1. It was observed that at a flow rate of 0.25 ml/sec (Q1), the shear stress is higher at the first port compared to the last port. However, in totality the shear stress exhibited by using a cannula side ported needle is less than that of a standard cannula needle.

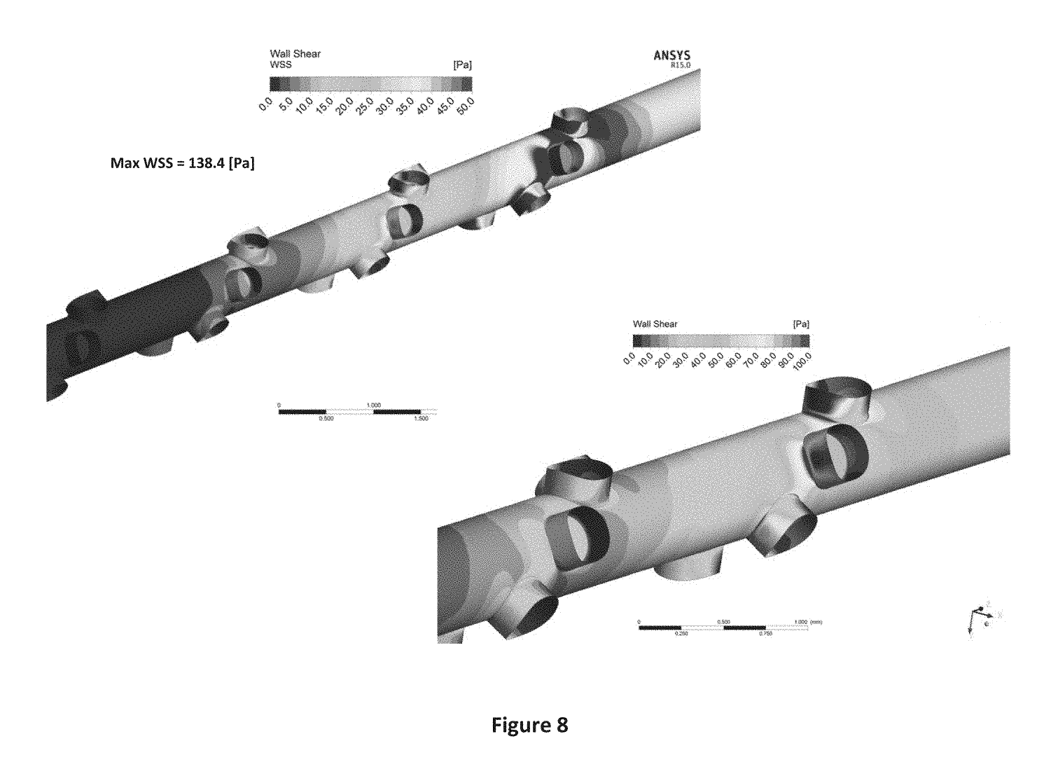

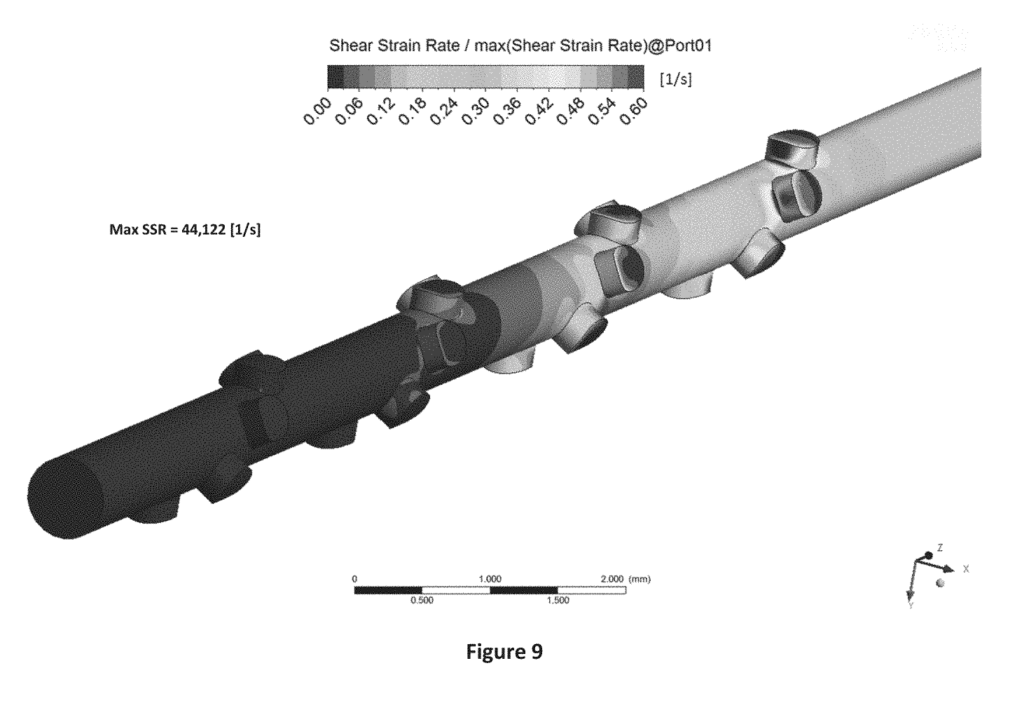

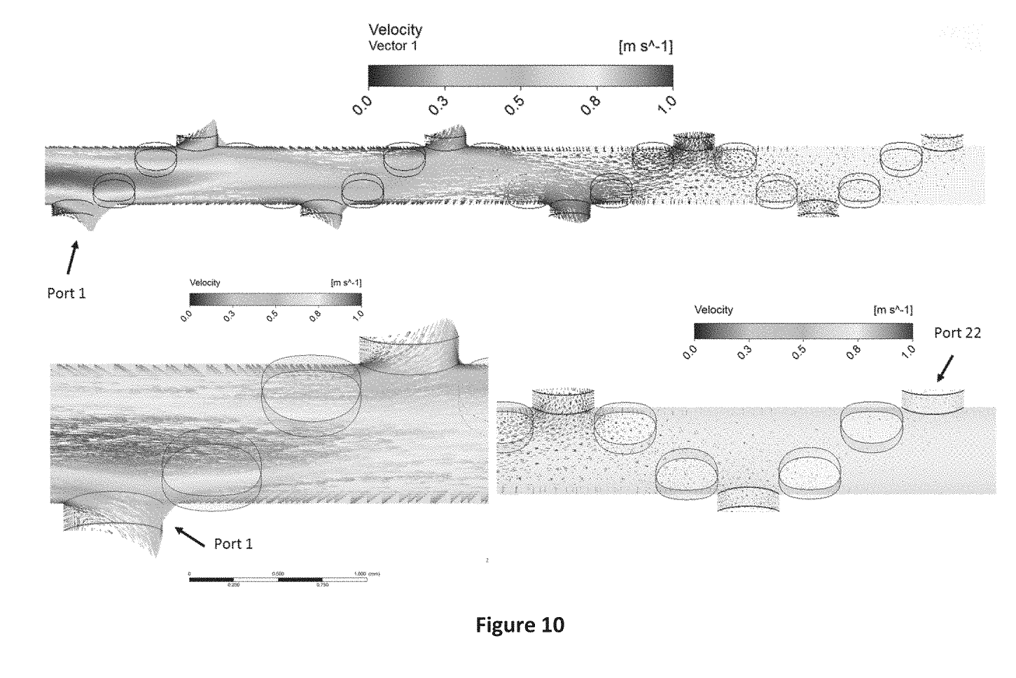

Examination of wall shear stress was also performed is shown in FIG. 8. As observed, the wall shear stress at port one is at 138.4 Pa and is substantially less than that seen for a standard cannula needle (396.3 Pa) The shear strain rate as shown in FIG. 9, also shows the advantage of using a cannula side ported needle at a flow rate of 0.25 ml/sec as the max shear strain rate is at 44.122.4 (1/sec) as compared to the shear strain rate, shown in FIG. 6, when using a standard cannula needle (89,762.1 (1/s)).

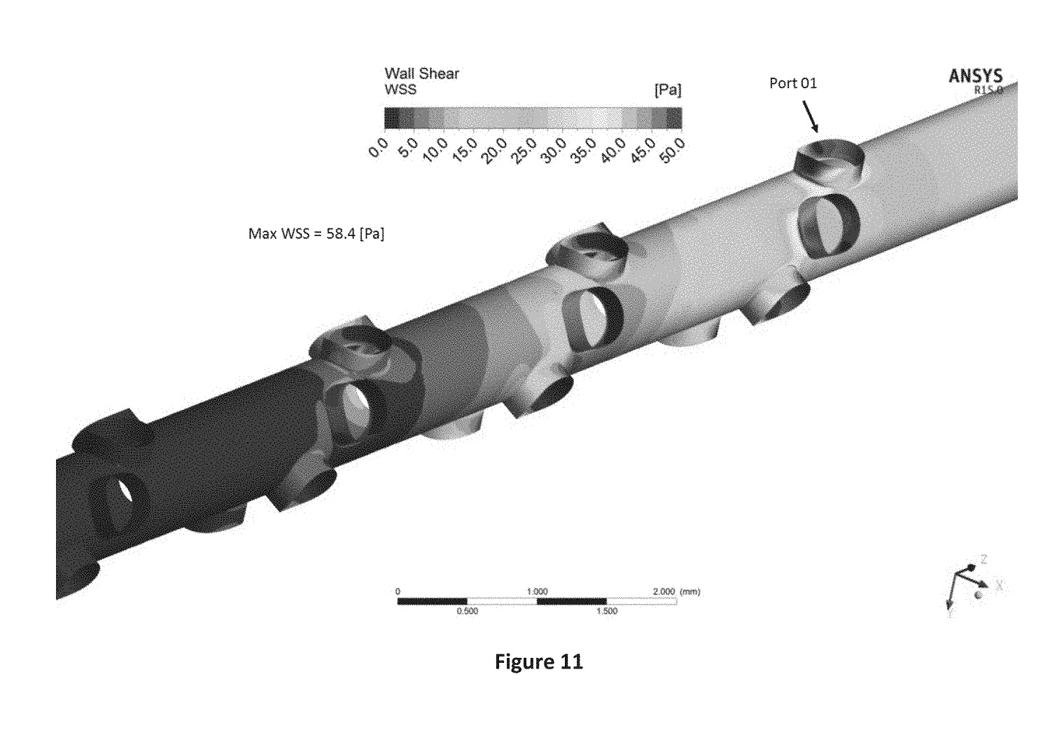

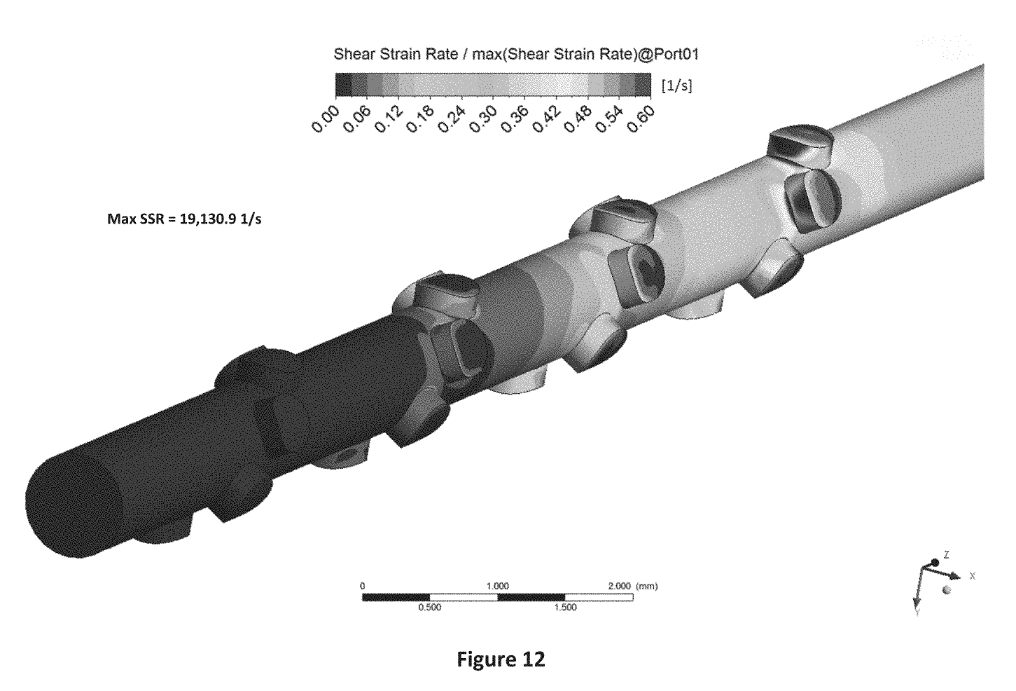

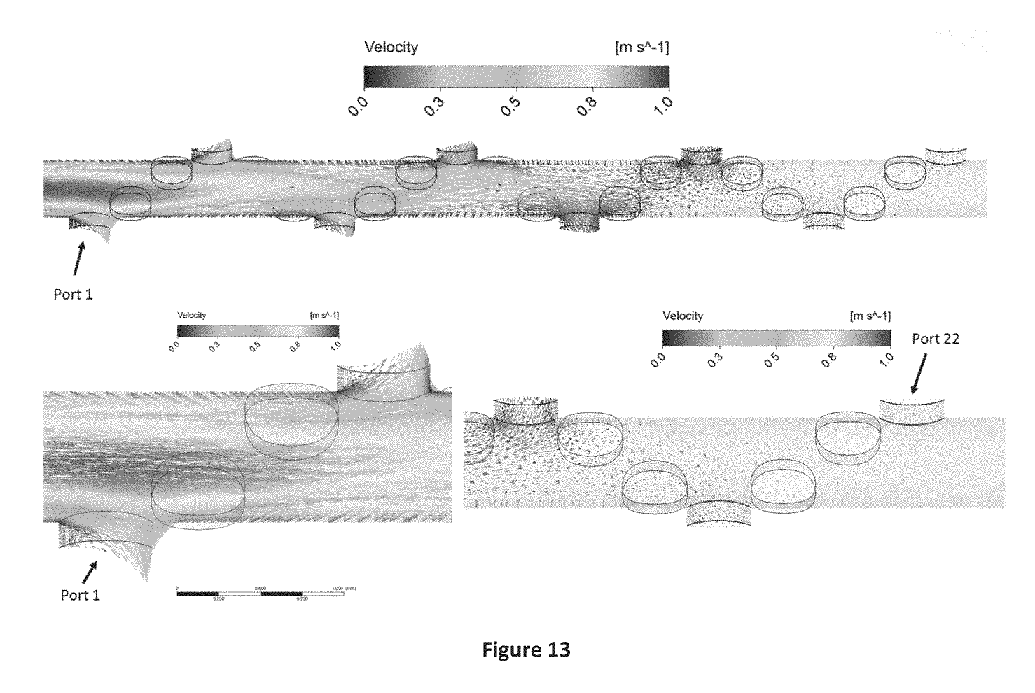

The fluid flow was modelled through needles, TIN-21-5.0 and TIN-19-7.5, at a rate of 0.25 ml/sec and the velocity vector is computationally measured. Measurement of the wall shear stress observed with TIN-19-7.5 shown in FIG. 11, is substantially less when compared to the wall shear stress exhibited when using the TIN-21-5.0 needle at the same flow rate. Measurement of the shear strain rate is also performed, shown in FIG. 12, and is substantially less when compared to the max shear rate run at the same flow rate using a TIN-21-5.0 needle (Max shear strain rate at first port, TIN-19-7.5=19,130.9 (1/s); TIN-21-5.0=44,122.4 (1/s (See FIG. 9). As expected, with a side ported design, the longer needle with bigger diameter affects the shear rate or stress less than the narrower needle. For a given flow rate, the longer lumen length may have a longer exposure to the cells that may be offset by lower shear rate and stress due to wider fluid path.

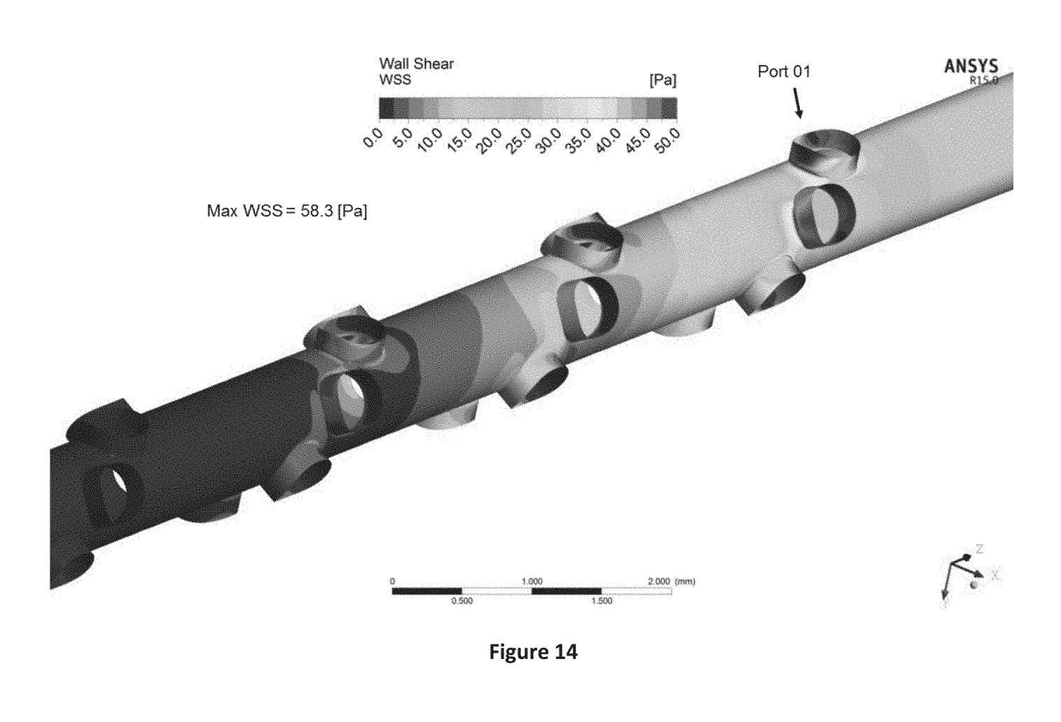

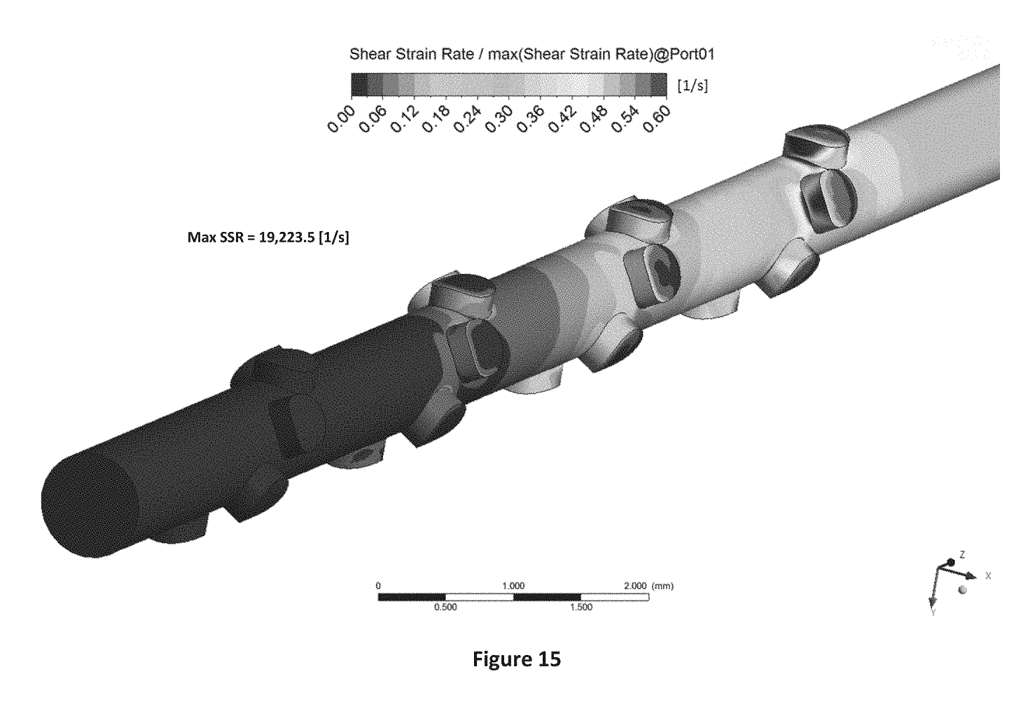

The fluid flow was modelled through needle at a rate of 0.25 ml/sec, in which the velocity vector was computationally measured. The wall shear stress was measured at 58.3 Pa, shown in FIG. 14, and was substantially less when compared to the wall shear stress at the same flow rate using a TIN-21-5.0 needle (see FIG. 8) but comparable to the wall shear stress to that of the TIN-19-7.5 needle. The shear strain rate was also measured and showed similar results, as shown in FIG. 15.

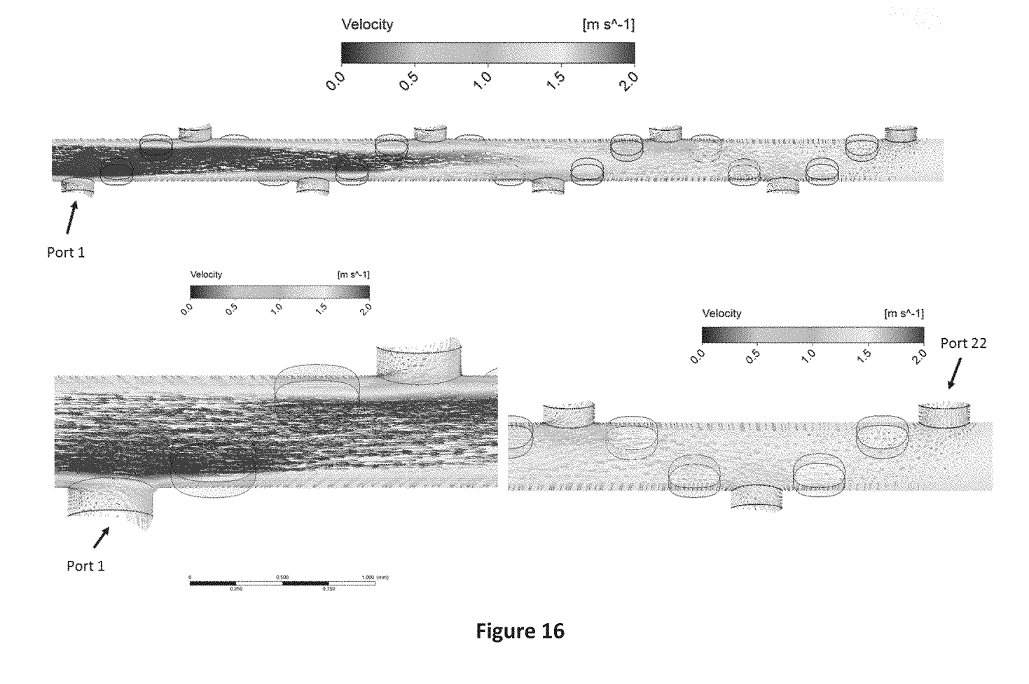

A computational assessment of cannula side ported needles was carried out using ANSYS CFD. For the testing fluid, a non-Newtonian mixed material (10% hematocrit blood model, 90% Carreau Yasuda blood model, with an average viscosity of 3.5 cP) was used for the sample and the needle model used was TIN-21-5.0 at a flow rate of 0.50 ml/sec (Q2). The properties of side ported needle model TIN-21-5.0 are shown in Table 1. As shown in FIG. 16, is the velocity vector using a flow rate of 0.50 ml/sec (Q2) from the first port of the cannula side ported needle to the twenty second port on the cannula side ported needle. As shown, shear stress is higher at the first port, however the shear stress exhibited by using a cannula side ported needle is less than that of a standard cannula needle.

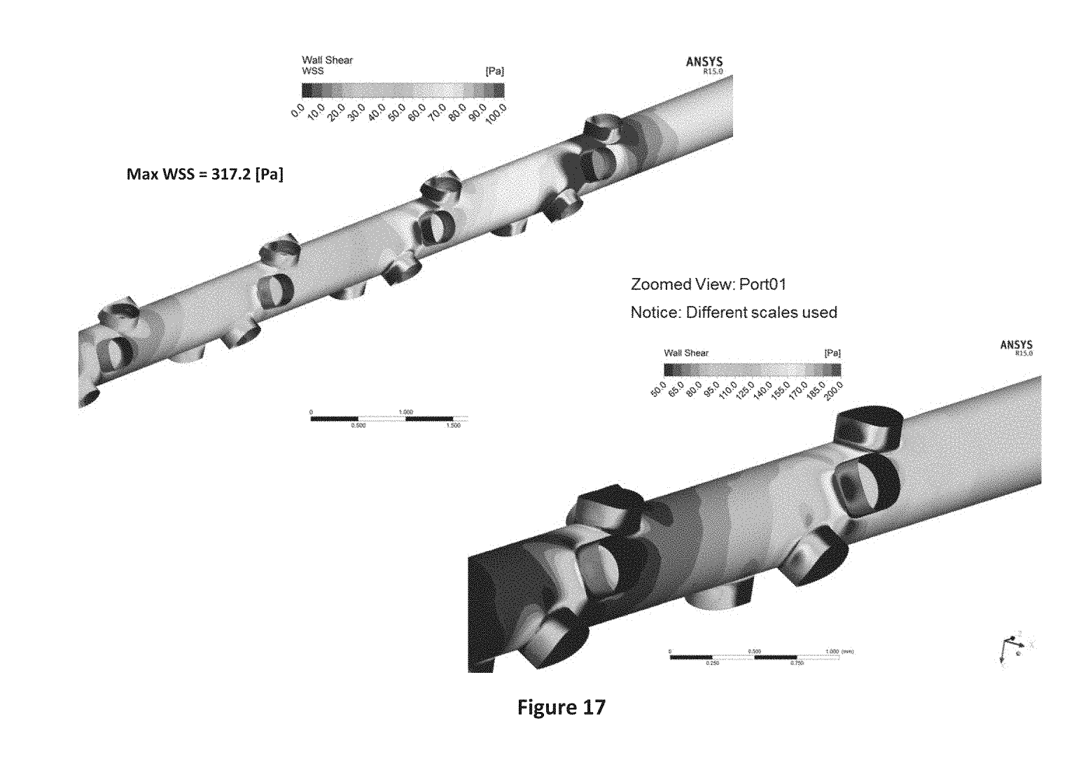

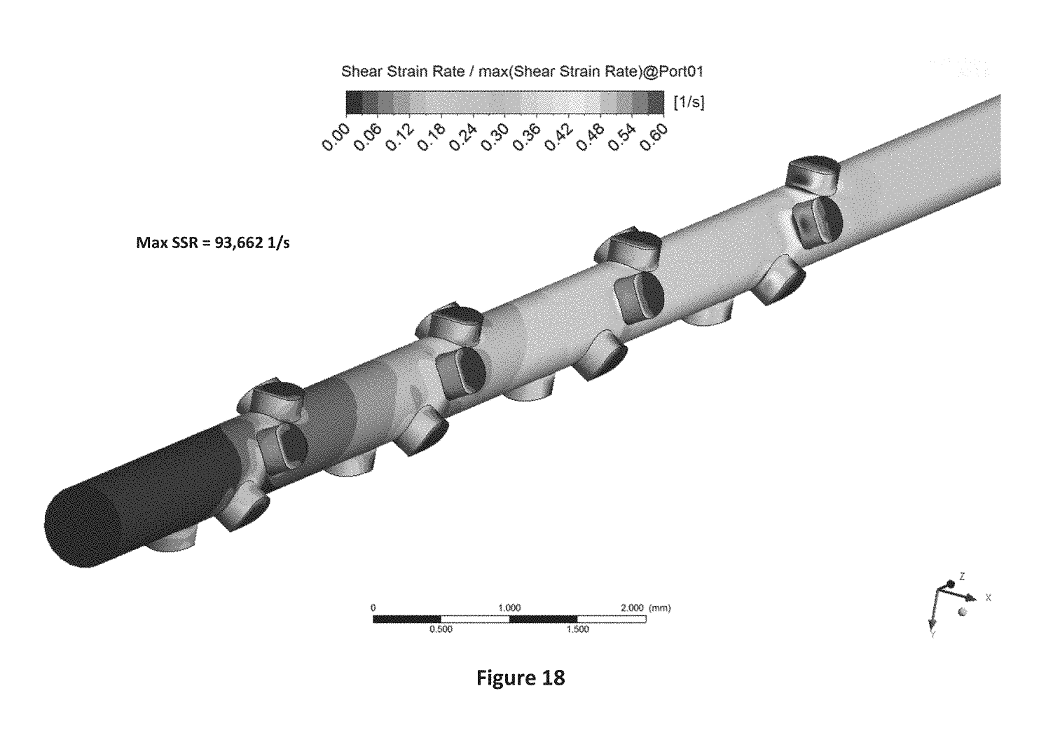

Examination of wall shear stress is shown in FIG. 17. As seen, the wall shear stress is at 317.2 Pa, which is substantially higher than that seen for the same needle in which the flow rate was set at 0.25 ml/sec (FIG. 8). The shear strain rate as shown in FIG. 18, also shows the advantage of using a lower flow rate of Q1 (0.25 ml/sec) as the max shear strain rate is at 93,622.5 (1/s) when the fluid flow rate is at Q2 (0.5 ml/sec) as compared 44,122.4 (1/sec) as shown in FIG. 9.

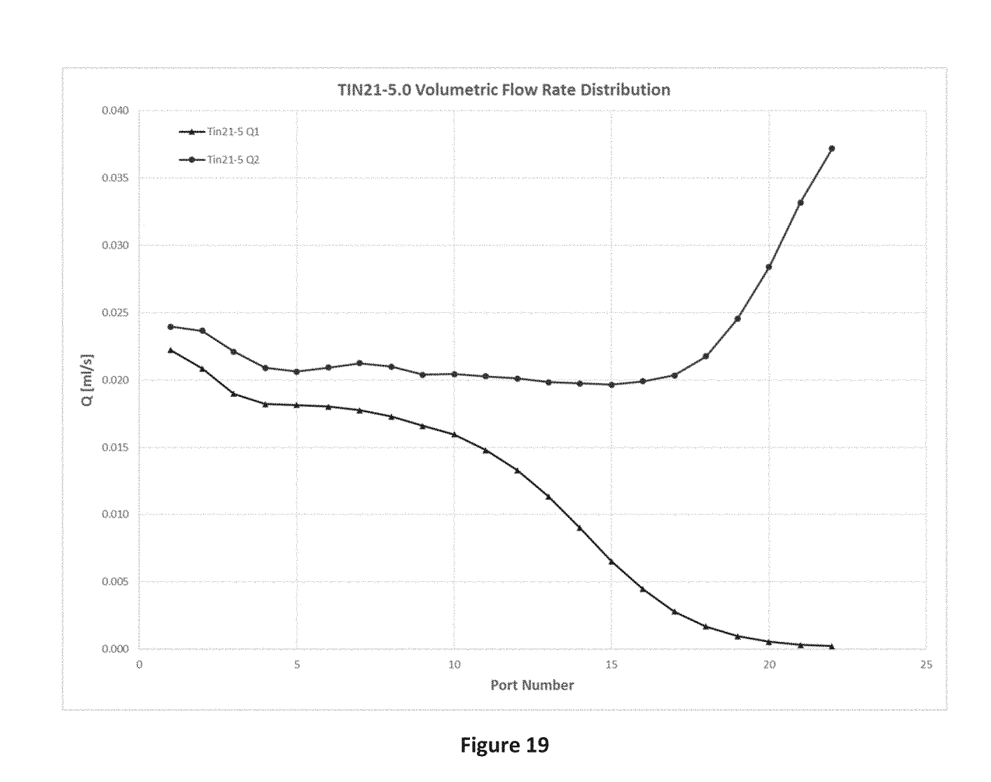

The volumetric flow rate distribution between the flow rates of Q1 and Q2 were then compared. As shown in FIG. 19, is the volumetric flow rate distribution over the 22 side ports. As indicated, at a higher flow rate, the flow rate increases at the most terminal side port (side port 22), whereas, at a flow rate of 0.25 ml/sec (Q1), the flow rate at the side port 22 has decreased.

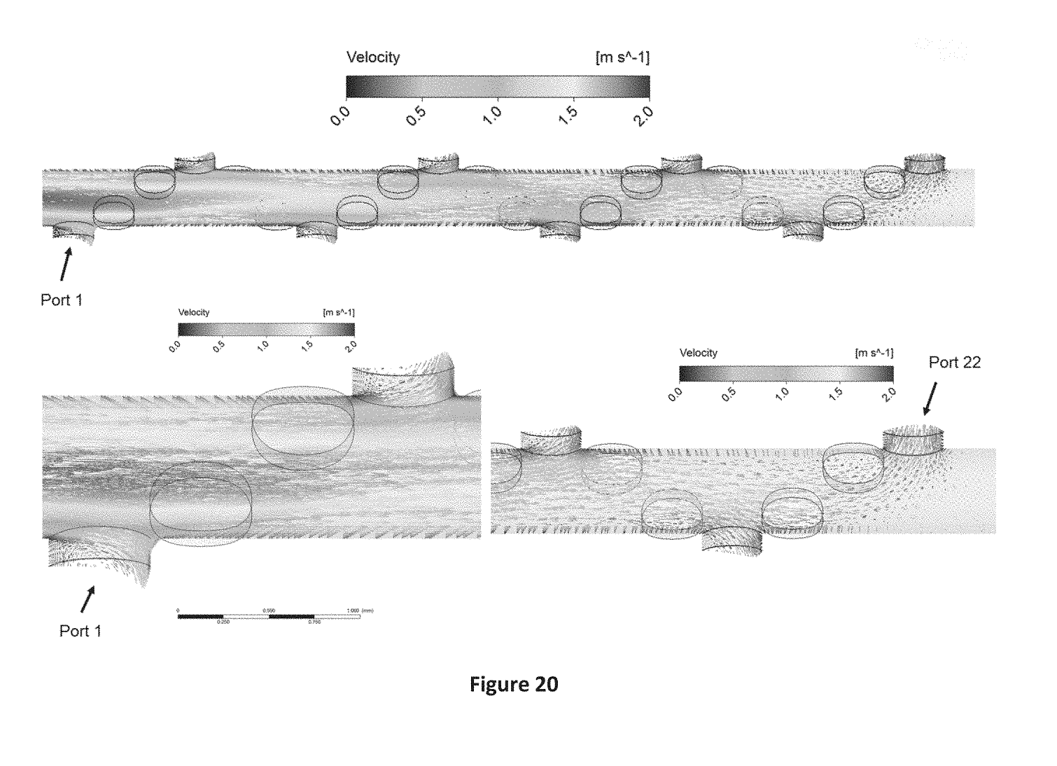

A computational assessment of cannula side ported needles was carried out using ANSYS CFD. For the testing fluid, a non-Newtonian mixed material (10% hematocrit blood model, 90% Carreau Yasuda blood model, with an average viscosity of 3.5 cP) was used for the sample and the needle model used was TIN-19-7.5 at a flow rate of 0.50 ml/sec (Q2). The properties of side ported needle model TIN-19-5.0 are shown in Table 1. As shown in FIG. 20, is the velocity vector using a flow rate of 0.50 ml/sec (Q2) from the first port of the cannula side ported needle to twenty second port on the cannula side ported needle. As shown, shear stress is higher at the first port, however the shear stress exhibited by using a cannula side ported needle is less than that of a standard cannula needle.

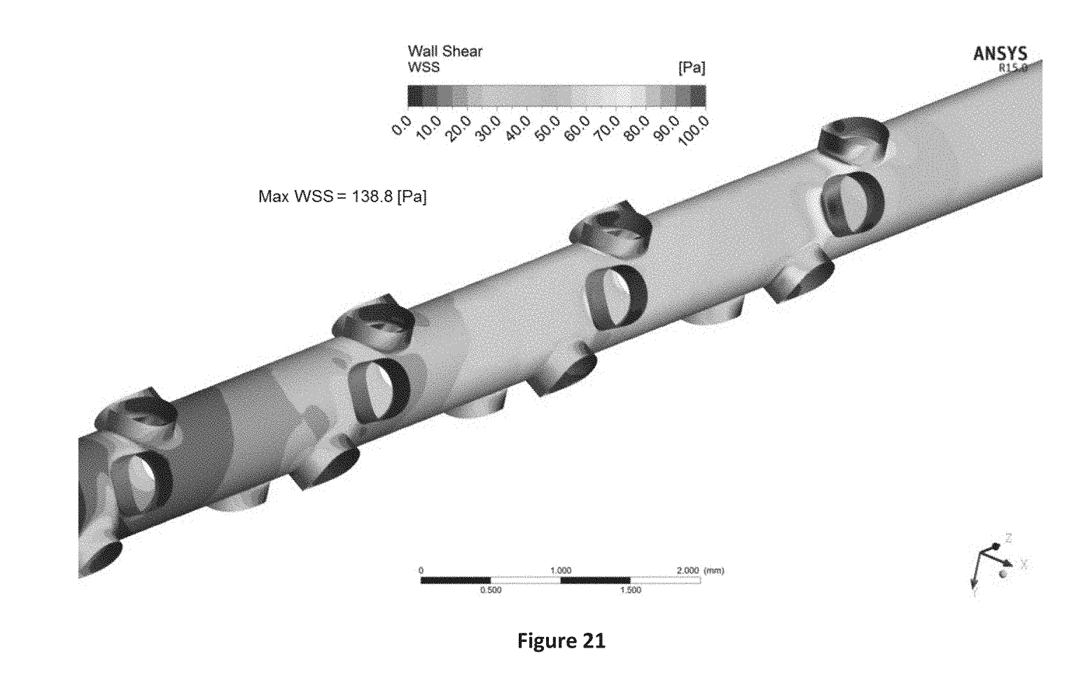

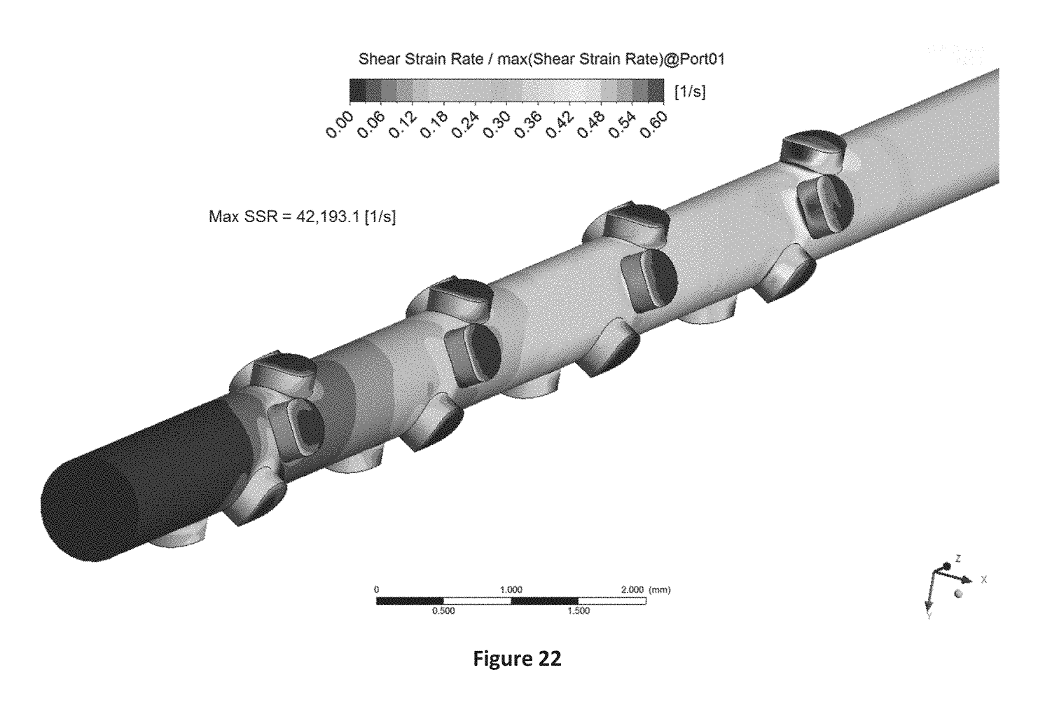

Examination of wall shear stress is shown in FIG. 21. As seen, the wall shear stress is at 138.8 Pa, which is similar to wall shear stress the flow rate was set at 0.25 ml/sec with a TIN-19-7.5 needle (FIG. 8). The shear strain rate as shown in FIG. 22, also shows the advantage of using a lower flow rate of Q1 (0.25 ml/sec) as the max shear strain rate is at 44,193.1 (1/s) when the fluid flow rate is at Q2 (0.5 ml/sec).

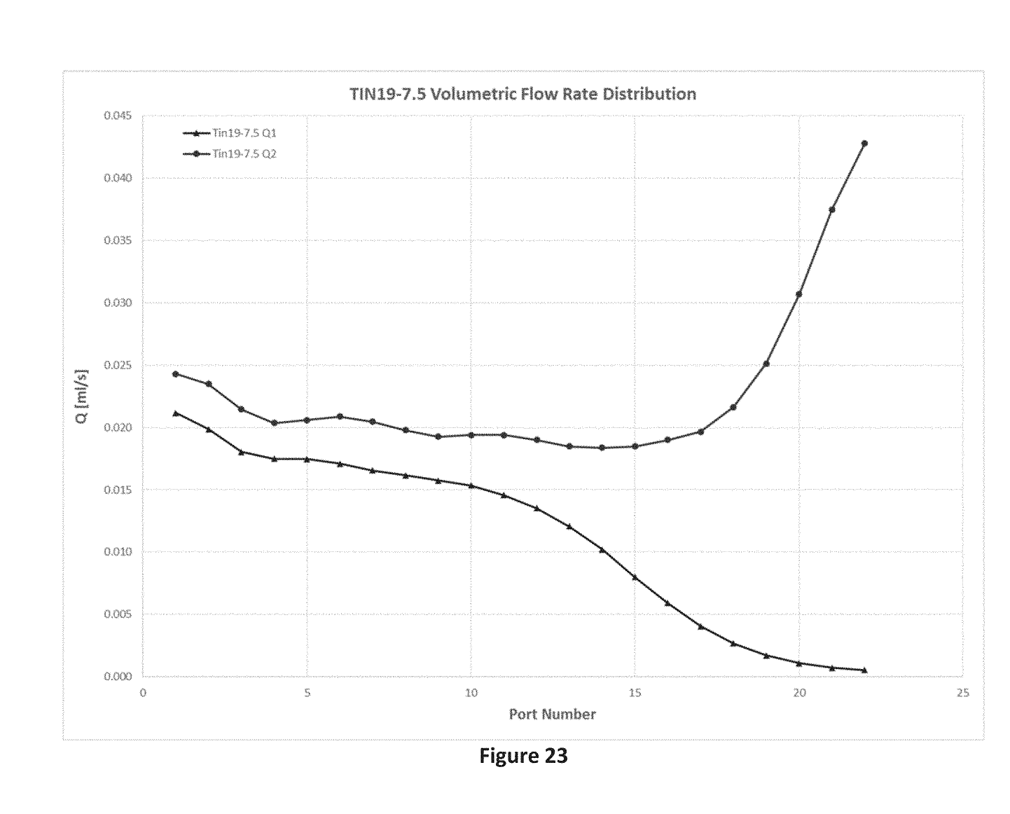

The volumetric flow rate distribution between the flow rates of Q1 and Q2 were then compared for the same needle. As shown in FIG. 23, is the volumetric flow rate distribution over the 22 side ports. As indicated, at a higher flow rate, the flow rate increases at the most terminal side port (side port 22), whereas, at a flow rate of 0.25 ml/sec (Q1), the flow rate at the side port 22 has decreased.

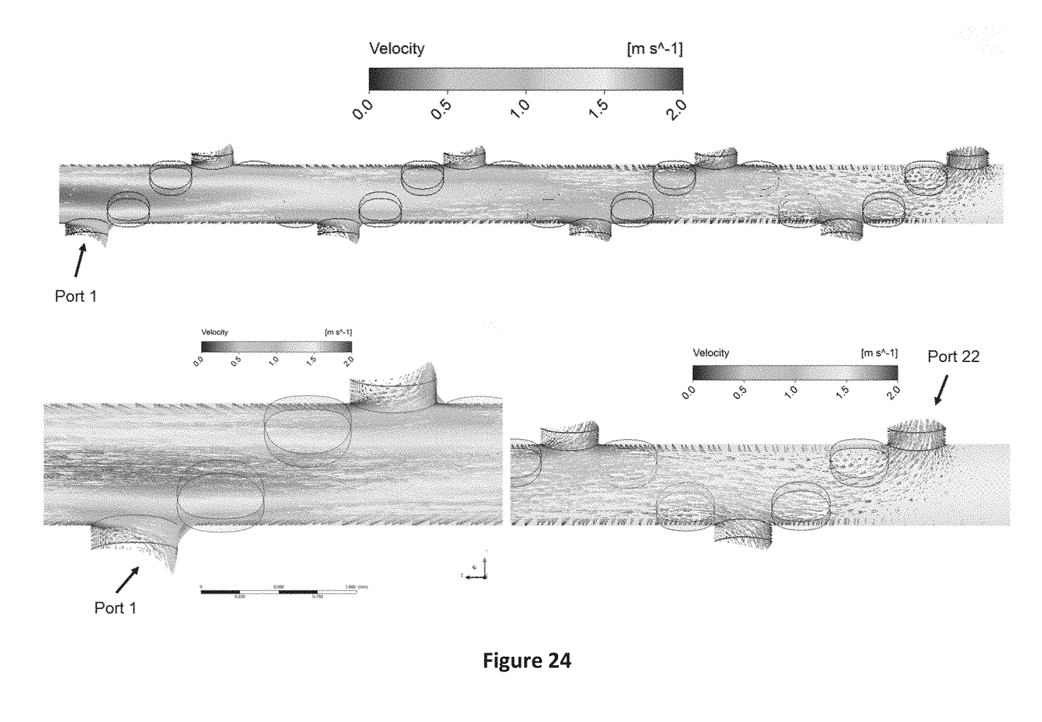

A computational assessment of cannula side ported needles was carried out using ANSYS CFD. For the testing fluid, a non-Newtonian mixed material (10% hematocrit blood model, 90% Carreau Yasuda blood model, with an average viscosity of 3.5 cP) was used for the sample and the needle model used was TIN-19-7.5 at a flow rate of 0.50 ml/sec (Q2). The properties of side ported needle model TIN-21-5.0 are shown in Table 1. As shown in FIG. 24, is the velocity vector using a flow rate of 0.50 ml/sec (Q2) from the first port of the cannula side ported needle to twenty second port on the cannula side ported needle. As shown, shear stress is higher at the first port, however the shear stress exhibited by using a cannula side ported needle is less than that of a standard cannula needle.

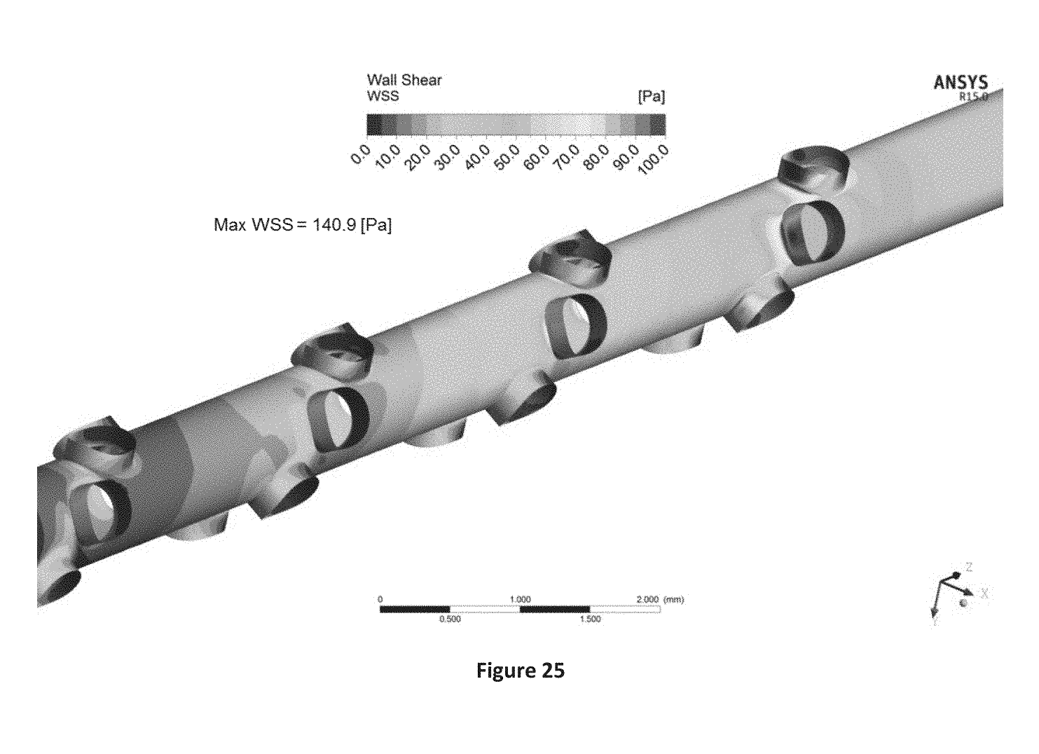

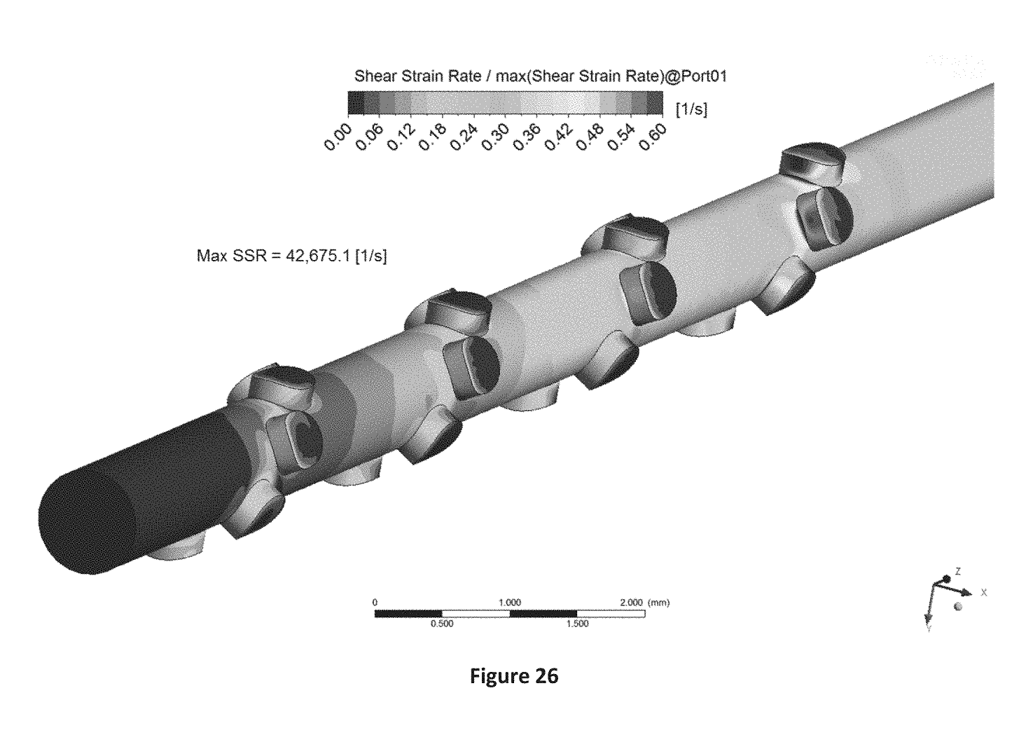

Examination of wall shear stress is shown in FIG. 25. As seen, the wall shear stress is at 140.9 Pa and the shear strain rate is shown in FIG. 26, at 42,675.1 (1/s).

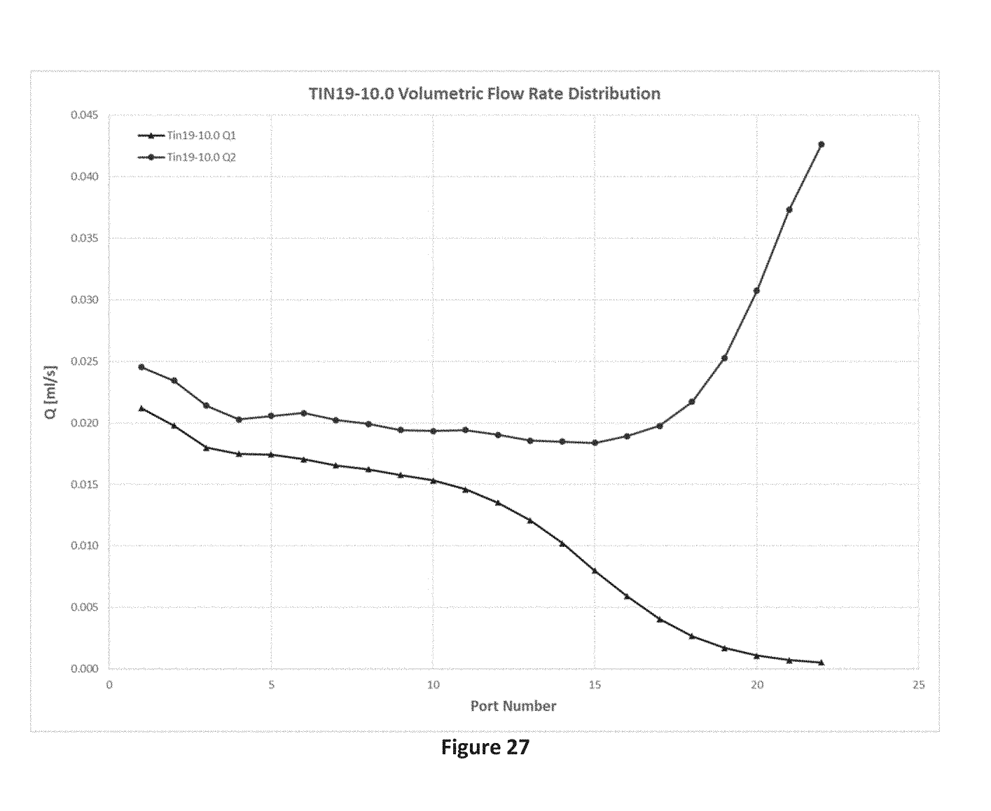

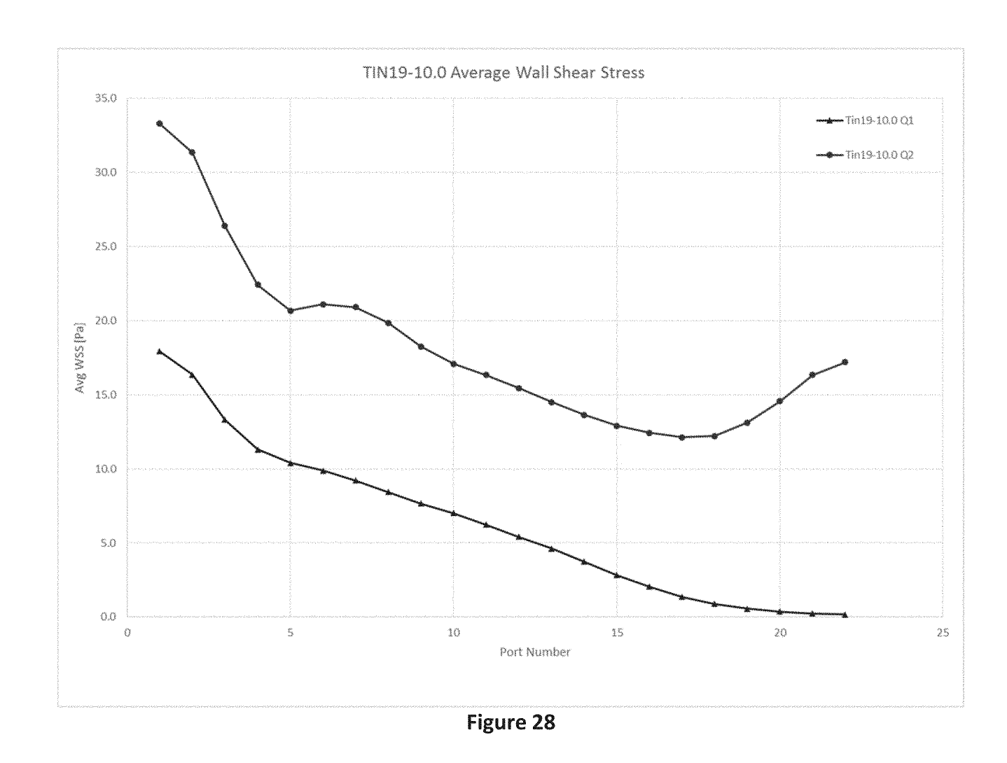

The volumetric flow rate distribution between the flow rates of Q1 and Q2 were then compared for the same needle. As shown in FIG. 27, is the volumetric flow rate distribution over the 22 side ports. As indicated, at a higher flow rate, the flow rate increases at the most terminal side port (side port 22), whereas, at a flow rate of 0.25 ml/sec (Q1), the flow rate at the side port 22 has decreased. As shown in FIG. 28, although the wall shear stress decreases towards the most terminal side port, the wall shear stress remains much lower, when the flow rate at 0.25 ml/sec is used.

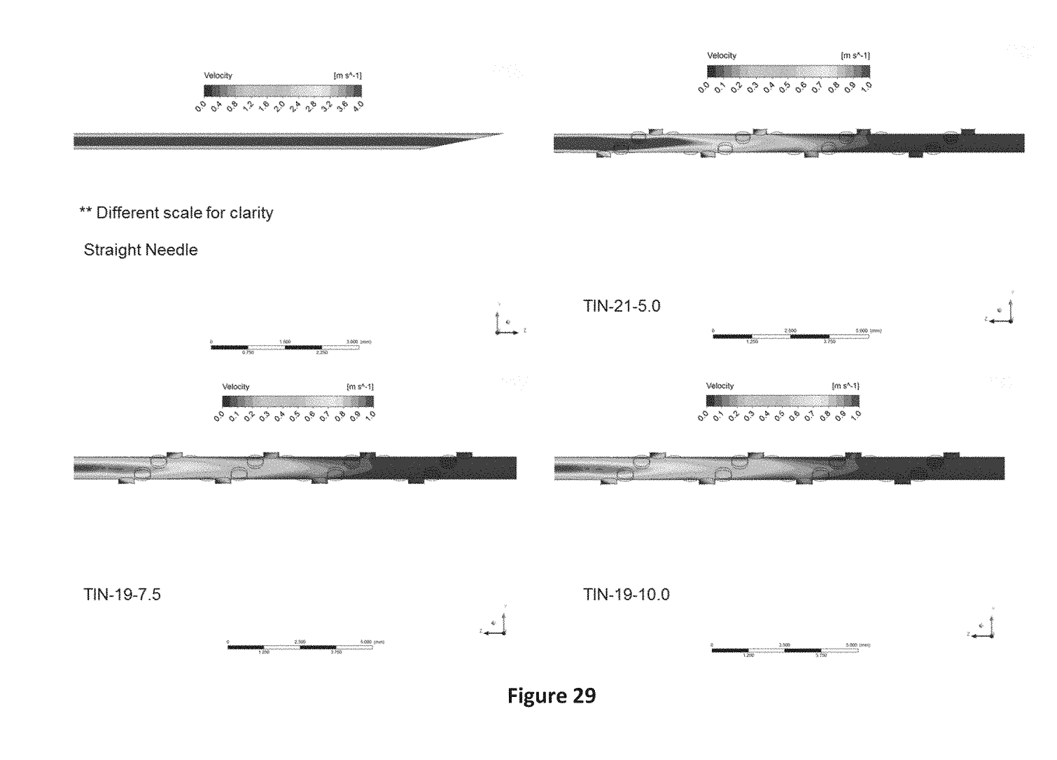

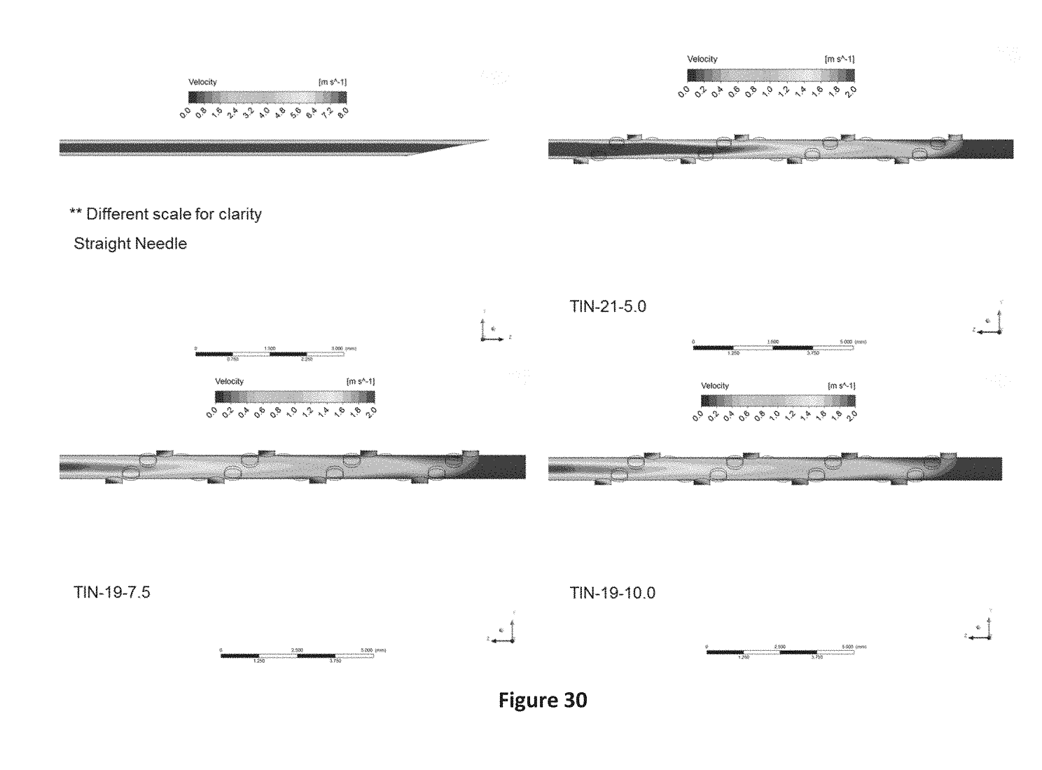

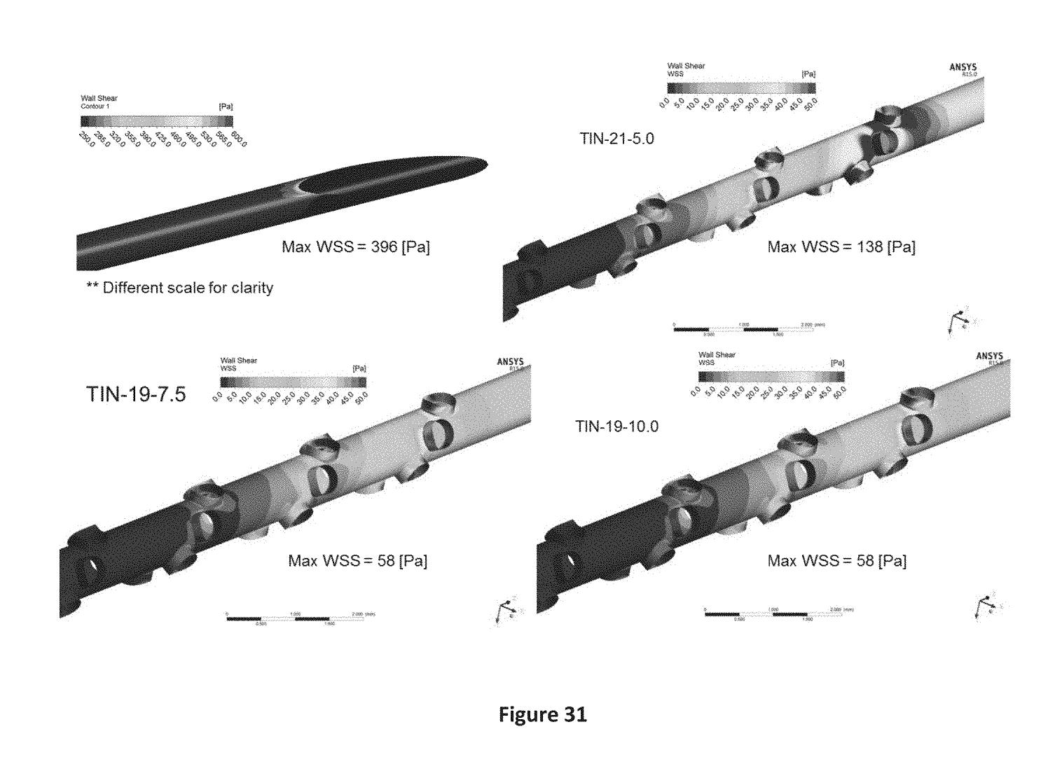

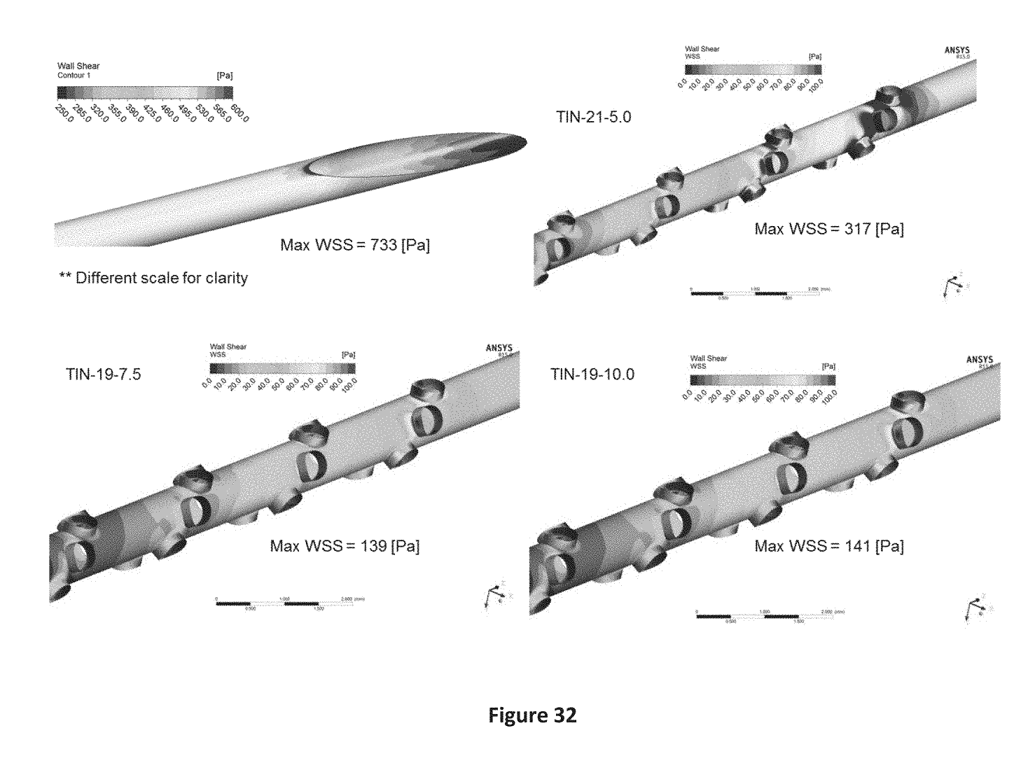

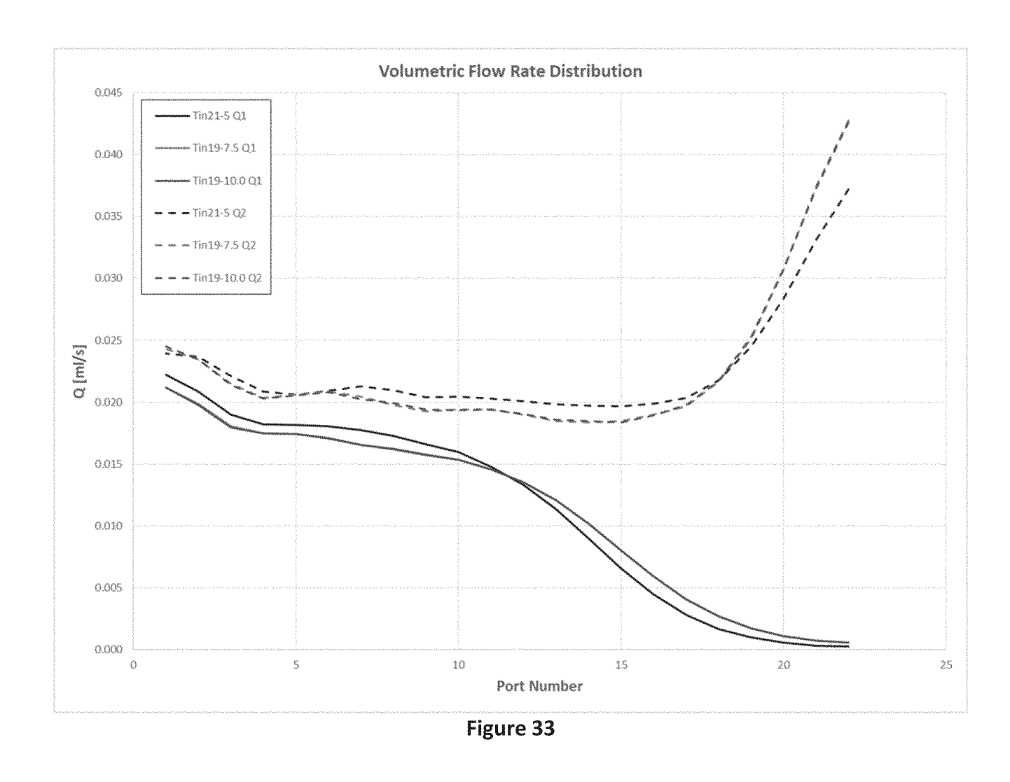

In summary, the computational tests examining the different flow rates of the Non-Newtonian mixed material model at Q1 (0.25 ml/sec) and Q2 (0.50 ml/sec) are shown using different cannula needles such as standard cannula needles and cannula side port needles with different properties as shown in Table 1. As shown in FIGS. 29 and 30 are the comparisons of the velocity contours at Q1 and Q2 respectively. As indicated, the velocity decreases towards the termini of the needles for all the side ported cannula needles. In comparisons of the shear wall stress for Q1 and Q2, FIGS. 31 and 32, respectively, it has increased drastically at a rate of Q2 among all the different needle types. A summary of the volumetric flow rate is shown in FIG. 33. As shown for use of the cannula side ported needles (TIN-21-5.0, TIN-19-7.5, TIN-19-10.0), the volumetric flow rate decreases towards the most distal side port (side port 22) when the flow rate is set at 0.25 ml/sec (Q1). However, at Q2 (0.5 ml/sec), the volumetric flow rate increases at the terminal side port. This coincides with the increase of wall shear stress increase seen at the flow rate Q2.

### Example 3. Post-Needle Characterization of Cellular Composition

Post-needle characterization of the cellular product was performed within 36 hours from bone-marrow aspiration. Statistical analysis of CFU-H data in aBMC, Post TIN and Post SHN samples was performed. Using one ANOVA aBMC vs Post TIN and aBMC SHN did not show any significant difference which indicates that there is no effect of shear on aBMC samples when passed through a needle at a flow rate of 0.25 ml/sec. For the experiment, 3 mL of the sample was passed through respective needle. The flow rate was performed manually with calibrated timer. Shown below in Table 2 are the WBC count data in aBMC, Post-TIN and Post-SHN samples. The testing was performed <3 hrs from bone-marrow aspiration.

TABLE 2
WBC count data in aBMC, post-TIN and post-SHN samples.

| | WBC Counts (×10⁶ per mL) | | |
|---|---|---|---|
| Sample ID | aBMC | Post-TIN | Post-SHN |
| BM0008 | 52.5 | 52.5 | 51.2 |
| BM0009 | 64.4 | 62.7 | 63.9 |
| BM0010 | 70.7 | 74.1 | 73.4 |
| BM0011 | 72.0 | 75.8 | 69.8 |
| BM0012 | 68.1 | 69.4 | 70.2 |
| BM0013 | 76.8 | 78.6 | 80.4 |
| BM0014 | 56.7 | 57.8 | 58.2 |
| BM0015 | 48.7 | 51.8 | 51.5 |
| BM0016 | 55.2 | 58.7 | 57.2 |
| BM0017 | 114.4 | 124.3 | 126.3 |

A summary of WBC count data in aBMC, Post-TIN and Post-SHN samples is shown below in Table 3. The testing was performed <3 hrs from bone-marrow aspiration.

TABLE 3
Summary of WBC count data in aBMC,
Post-TIN and Post-SHN samples.

| | | Post-TIN | Post-SHN |
|---|---|---|---|
| | aBMC | (21 g, 5 cm) | (23 g, 2.5 cm) |
| Number of values | 10 | 10 | 10 |
| Minimum | 48.70 | 51.80 | 51.20 |
| 25% Percentile | 54.53 | 56.48 | 55.78 |
| Median | 66.25 | 66.05 | 66.85 |
| 75% Percentile | 73.20 | 76.50 | 75.15 |
| Maximum | 114.4 | 124.3 | 126.3 |
| Mean | 67.95 | 70.57 | 70.21 |
| Std. Deviation | 18.77 | 21.18 | 21.92 |
| Std. Error of Mean | 5.936 | 6.698 | 6.932 |
| Lower 95% CI of mean | 54.52 | 55.42 | 54.53 |
| Upper 95% CI of mean | 81.38 | 85.72 | 85.89 |

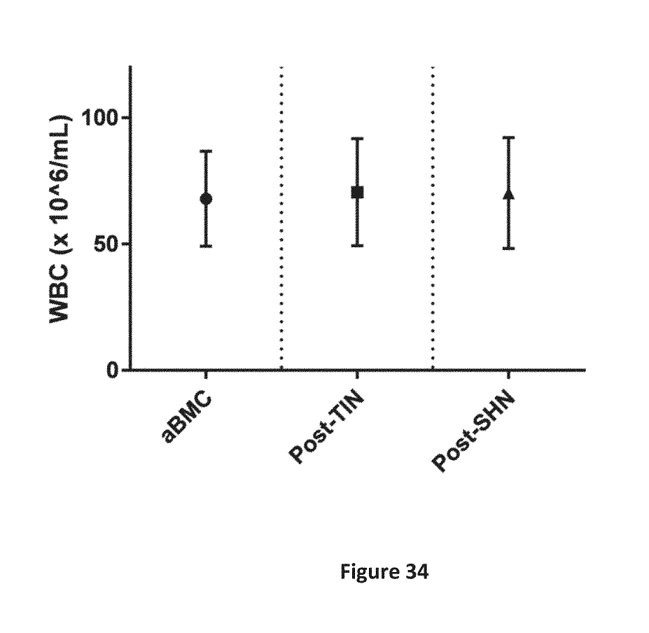

As shown in FIG. 34, for the WBC data, is a graphical presentation of one ANOVA (Dunnett's multiple comparisons test). aBMC vs Post-TIN and aBMC vs Post-SHN which did not show significant differences which is indicative of no effect of shear on the samples when the flow rate is 0.25 ml/s.

Data was also collected on CD34⁺ cells. As shown below is the CFU-H data (CFU/50000 WBC) in aBMC, Post-TIN and Post-SHN samples in Table 4 from 10 samples. The testing was performed <36 hrs from bone-marrow aspiration.

TABLE 4
CFU-H data (CFU/50000 WBC) in aBMC,
Post-TIN and Post-SHN samples.

| | CFU-H/50000 WBC | | |
|---|---|---|---|
| | | Post-TIN | Post-SHN |
| Sample ID | aBMC | (21 g, 5 cm) | Sample |
| BM0008 | 106 | 160 | 127 |
| BM0009 | 105 | 99 | 108 |
| BM0010 | 113 | 146 | 136 |
| BM0011 | 148 | 159 | 175 |
| BM0012 | 184 | 184 | 181 |
| BM0014 | 114 | 137 | 148 |
| BM0015 | 122 | 114 | 118 |
| BM0016 | 175 | 124 | 178 |
| BM0017 | 131 | 136 | 130 |

A summary of CFU-H data (CFU/50000 WBC) data in aBMC, Post TIN and Post-SHN samples is shown in Table 5, below. The testing was performed <36 hrs from bone-marrow aspiration.

TABLE 5
Summary of CFU-H data (CFU/50000 WBC) data
in aBMC, Post-TIN and Post-SHN samples.

| | | Post-TIN | Post-SHN |
|---|---|---|---|
| | aBMC | (21 g, 5 cm) | (23 g, 2.5 cm) |
| Number of values | 9 | 9 | 9 |
| Minimum | 105.0 | 99.00 | 108.0 |
| 25% Percentile | 109.5 | 119.0 | 122.5 |
| Median | 122.0 | 137.0 | 136.0 |
| 75% Percentile | 161.5 | 159.5 | 176.5 |
| Maximum | 184.0 | 184.0 | 181.0 |
| Mean | 133.1 | 139.9 | 144.6 |
| Std. Deviation | 29.56 | 25.91 | 27.43 |
| Std. Error of Mean | 9.852 | 8.637 | 9.144 |
| Lower 95% CI of mean | 110.4 | 120.0 | 123.5 |
| Upper 95% CI of mean | 155.8 | 159.8 | 165.6 |

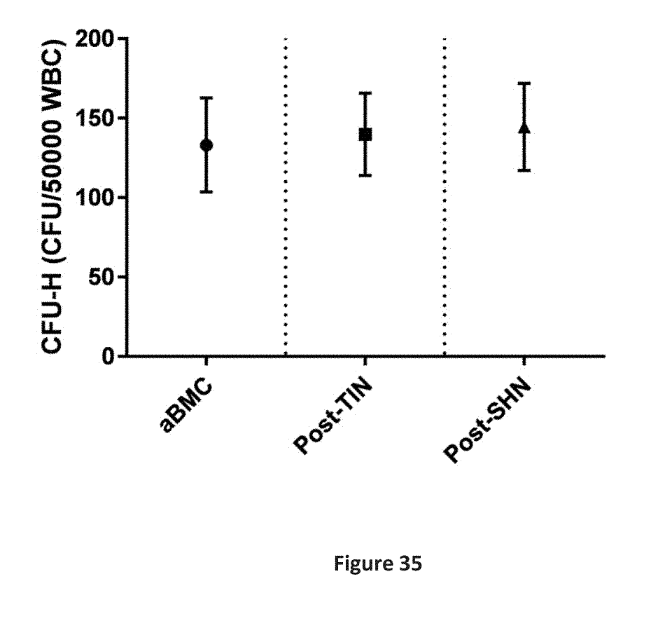

As shown in FIG. 35, for the CFU-H data, is the graphical presentation of one ANOVA (Dunnett's multiple comparisons test). aBMC vs Post-TIN and aBMC vs Post-SHN did not show any significant difference indicating no effect of shear on aBMC samples when passed through needle at a flow-rate of 0.25 mL/s.

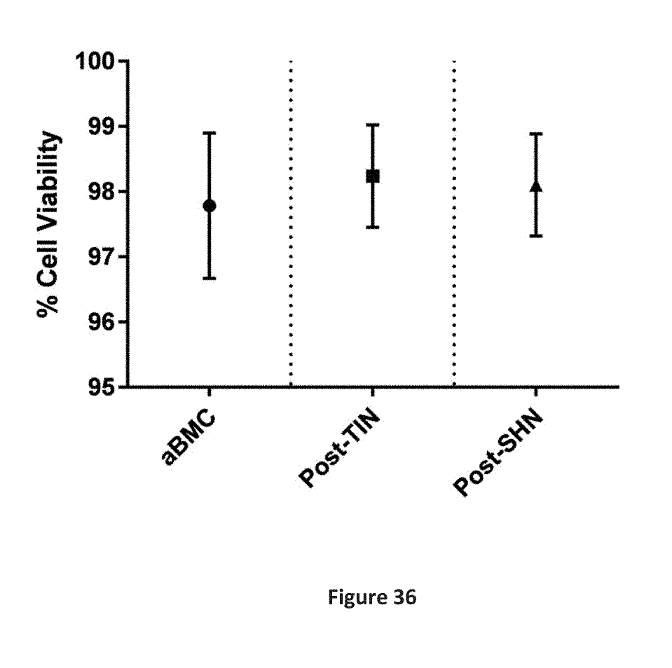

As shown in FIG. 36, for the % cell viability data, is the graphical presentation of one ANOVA statistical analysis (Dunnett's multiple comparisons test). aBMC vs Post-TIN and aBMC vs Post-SHN did not show any significant difference indicating no effect of shear on aBMC samples when passed through needle at a flow-rate of 0.25 mL/s.

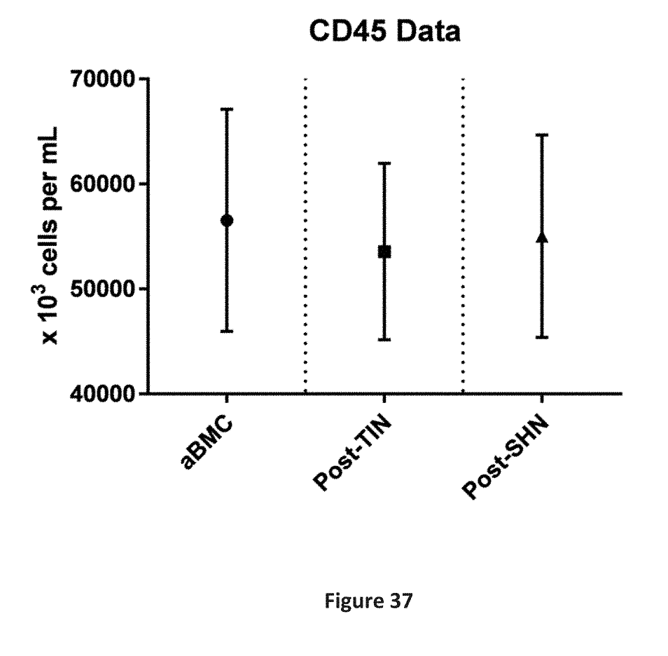

As shown in FIG. 37, for the viable CD45+ cell count data, is the graphical presentation of one ANOVA statistical analysis (Dunnett's multiple comparisons test). aBMC vs Post-TIN and aBMC vs Post-SHN did not show any significant difference indicating no effect of shear on aBMC samples when passed through needle at a flow-rate of 0.25 mL/s.

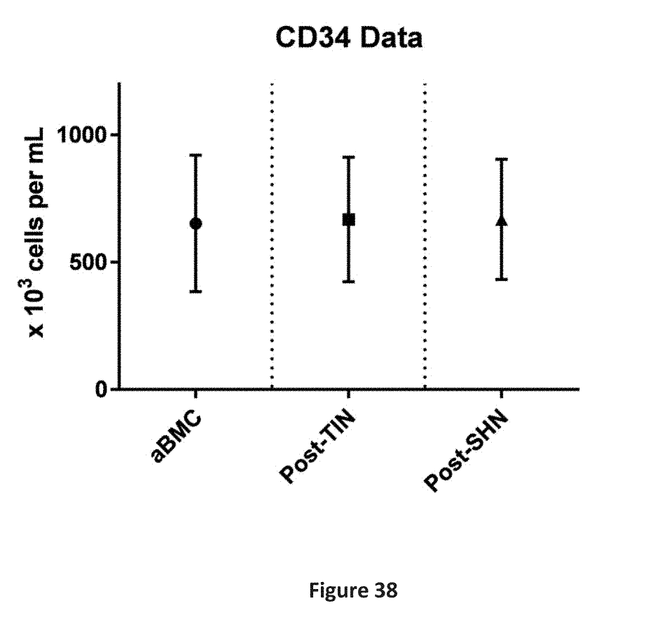

As shown in FIG. 38, for the viable CD34+ cell count data, is the graphical presentation of one ANOVA statistical analysis (Dunnett's multiple comparisons test). aBMC vs Post-TIN and aBMC vs Post-SHN did not show any significant difference indicating no effect of shear on aBMC samples when passed through needle at a flow-rate of 0.25 mL/s.

### Example 4. Composition of the Cells in an Exemplary Product

A composition for treatment was generated from human bone marrow aspirate of 120 mL, which was concentrated for a final volume of 20 milliliters. The types of cells are shown in Table 6 below. As shown the composition comprises a mixture of stromal cells, mesenchymal cells, hematopoietic stem cells, and endothelial progenitor cells. Additionally, the composition comprised red blood cells and plasma. The composition had a hematocrit in a range of 4.3 to 10.9% (see Table 7 below).

TABLE 6
Dose of final product infused (20 mls)

| | Dose of the Final Product Infused (20 mls) | | | | | |
|---|---|---|---|---|---|---|
| | Cellularity | | | | Sterility | |
| | Total | Total | Viable | Potenc | Microbiological | |
| Subject | TNC | MNC | CD31 + ve | CFUs | testing USP | Biological |
| ID | (×10\hat{ }8) | (c10\hat{ }8) | (×10\hat{ }6) | (×10\hat{ }4) | <71> | Response |
| 1 | 3.50 | 1.87 | 0.62 | 29.40 | No Growth | Minor amputation |
| 2 | 3.54 | 2.31 | 3.72 | 22.66 | No Growth | No adverse events |
| 3 | 4.26 | 1.53 | 2.96 | 71.99 | No Growth | Death (unrelated to |
| | | | | | | the study |
| | | | | | | intervention) |
| 4 | 12.96 | 1.96 | 10.29 | 287.71 | No Growth | No adverse events |
| 5 | 7.28 | 1.09 | 5.61 | 19.66 | No Growth | Major amputation |
| 6 | 10.36 | 1.86 | 2.21 | 10.36 | No Growth | Major amputation |
| 7 | 10.54 | 1.37 | 3.23 | 47.43 | No Growth | Minor amputation |
| 8 | 13.46 | 4.30 | 10.33 | 110.37 | No Growth | No adverse events |
| 9 | 8.98 | 3.23 | 3.86 | 44.90 | No Growth | No adverse events |
| 10 | 7.34 | 0.73 | 2.28 | 160.75 | No Growth | Death (unrelated to |
| | | | | | | the study |
| | | | | | | intervention) |
| 11 | 5.86 | 1.92 | 1.05 | 36.33 | No Growth | No adverse events |
| 12 | 4.22 | 1.52 | 1.41 | 101.70 | No Growth | Major amputation |
| 13 | 6.28 | 2.45 | 0.53 | 21.98 | No Growth | No adverse events |
| 14 | 4.80 | 0.96 | 0.67 | 27.84 | No Growth | No adverse events |
| 15 | 15.34 | 3.84 | 13.53 | 392.70 | No Growth | No adverse events |
| 16 | 7.62 | 2.29 | 2.53 | 53.34 | No Growth | No adverse events |
| 17 | 10.36 | 3.50 | 4.09 | 279.61 | No Growth | No adverse events |
| Average | 8.04 | 2.16 | 4.05 | 101.10 | [symbol] | [symbol] |
| St. Dev. | 3.66 | 1.02 | 3.82 | 113.61 | [symbol] | [symbol] |
| Minimum | 3.50 | 0.73 | 0.53 | 10.36 | [symbol] | [symbol] |
| Maximum | 15.34 | 4.30 | 13.53 | 392.70 | [symbol] | [symbol] |

[symbol]

indicates data missing or illegible when filed

[symbol]

TABLE 7
Cell Sample Compositions

| | RBC count | Hgb | HCT |
|---|---|---|---|
| S. No. | (×10\hat{ }6/nL) | (gm/dL) | (%) |
| 1 | 0.74 | 3.20 | 7.00 |
| 2 | 1.05 | 3.40 | 8.10 |
| 3 | 1.20 | 4.20 | 10.60 |
| 4 | 0.45 | 1.60 | 4.30 |
| 5 | 0.65 | 2.00 | 5.70 |
| 6 | 1.28 | 3.80 | 8.60 |
| 7 | 1.11 | 3.50 | 9.10 |
| 8 | 1.07 | 3.90 | 9.30 |
| 9 | 1.02 | 4.00 | 9.40 |
| 10 | 1.21 | 3.80 | 9.60 |
| 11 | 1.02 | 3.90 | 9.30 |
| 12 | 1.07 | 3.10 | 9.60 |
| 13 | 1.23 | 3.50 | 10.90 |
| 14 | 1.14 | 3.30 | 9.40 |
| 15 | 1.02 | 3.30 | 8.20 |
| 16 | 0.07 | 3.10 | 7.50 |
| 17 | 1.43 | 2.90 | 9.40 |
| Average | 0.99 | 3.32 | 8.59 |
| SD | 0.34 | 0.68 | 1.69 |

### Example 5. Evaluation of Cell Viability with Different Amounts of Red Blood Cells in the Composition

An amount of bone marrow (e.g., 120-180 mL) is aspirated from a subject. The bone marrow aspirate is processed and concentrated to obtain an autologous bone marrow cell concentrate (aBMC) composition having a final volume of 20 milliliters by using a sterile, disposable bone marrow processing device. The aBMC composition is preferably devoid or substantially free from Red blood cells (RBCs). The population of total nucleated cells, total mononuclear cells, and WBCs of the aBMC is measured (e.g., by using Automated hemocytometer using a coulter principle). RBCs are added in the aBMC composition so as to reach a final hematocrit of the composition or a total cell fraction of the composition (total cell volume of the composition) of 3%, 5%, 10%, 15%, 20%, 25%, 30%, 35%, or 40%, or a total cell fraction of the composition (total cell volume of the composition) or final hematocrit that is within a range defined by any two of the aforementioned values. Autologous plasma is added to the aBMC composition to adjust the viscosity to a final value of 1.5-5.0 cP at 37° C. The resultant aBMC compositions comprising the RBCs and autologous plasma at a viscosity of 1.5-5.0 cP is then passed through a TIN side-ported needle at 0.25 mL/s. Within 24 hours after delivery of the cells through the needle, hemolysis (by measuring free hemoglobin in plasma fraction) and the cell viability and/or apoptosis of WBCs, CD45⁺ and CD34⁺ cells is measured (e.g., by using trypan blue exclusion assay, Annexin-V assay, or 7AAD/PI assay). An improved and better cell composition is expected when the final hematocrit of the composition is within the range of 2-20%.

### Example 6. Evaluation of Cell Viability with Different Flow Rates

An amount of bone marrow (e.g., 120-180 mL) is aspirated from a subject. The bone marrow aspirate is processed and concentrated to obtain an autologous bone marrow cell concentrate (aBMC) composition having a final volume of 20 milliliters by using a sterile, disposable bone marrow processing device. The aBMC composition is preferably devoid or substantially free from Red blood cells (RBCs). The population of total nucleated cells, total mononuclear cells, and WBCs of the aBMC is measured (e.g., by using Automated hemocytometer using a coulter principle). RBCs are added to the aBMC composition so as to reach a total cell fraction of the composition (total cell volume of the composition) of 20%. Autologous plasma is added to the aBMC composition to adjust the viscosity to a final value of 1.5-5.0 cP at 37° C. The resultant aBMC compositions are then passed through a TIN side-ported needle at a flow rate of 0.1 mL/s, 0.15 mL/s, mL/s, 0.25 mL/s, 0.3 mL/s, 0.35 mL/s, 0.4 mL/s, 0.45 mL/s and/or 0.5 mL/s or at a flow rate within a range defined by any two of the aforementioned values. Within 24 hours after delivery of the cells through the needle, hemolysis (by measuring free hemoglobin in plasma fraction) and the cell viability and/or apoptosis of WBCs, CD45⁺ and CD34⁺ cells is measured (e.g., by using trypan blue exclusion assay, Annexin-V assay, or 7AAD/PI assay). An improved cell composition is expected when the flow rate is within the range of 0.1 mL to 0.5 mL per second.

### Example 7. Results of a Research Study Conducted to Test the Ability of Bone Marrow Concentrate (BMC) to Induce Chemotactic Migration and “Tube Formation” of Human Umbilical Vein Endothelial Cells (HUVECs)

Autologous cell therapies are patient (biologically) unique in that the active component consists of living cells, which have mixed pharmacologic characteristics, and produce variable amounts of bioactive molecules with complex biochemical interactions.

Many cell types display the ability to home to particular regions of the body by responding to local environmental cues. For instance, Endothelial Progenitor Cells (EPCs), present in bone marrow (BM) and aBMC, are expected to respond to homing signals generated by hypoxic tissues.

Local injection of bone marrow-mononuclear cells (BM-MNCs) led to new vessel formation in both preclinical and clinical studies (Lee and Yoon, Br J Pharmacol. 2013; 169(2):290-303; incorporated by reference in its entirety herein). A plausible mechanism for such effects includes tropic factors secreted by the injected cells and acting on host cells, the latter of which then carrying out the actual vascularization. Additionally, some of the injected cells, especially in autologous settings, may engraft and thereby provide structural support for the new vessels.

Numerous secreted factors have been proposed to contribute to the formation of blood vessels including VEGF and SDF-1. Whereas VEGF supports multiple aspects of vessel formation, SDF-1 is primarily considered a factor promoting directed cell migration (Ho et al., Cardiol Res Pract. 2012; 2012:143209; incorporated by reference in its entirety herein). Endostatin, on the other hand, is an angiogenesis inhibitor.

The cellular component of the aBMC constitutes a heterogeneous mixture of Total Nucleated Cells (TNC) subpopulations including EPCs, mesenchymal stem cells (MSCs), and hematopoietic cells. In contrast, studies which have used single cell types, have not met efficacy endpoints.

The CD34⁺ fraction of the aBMC contains EPCs and is thus expected to contribute to vasculogenesis in the CLIRST III trial. However, Dubsky who studied no-option diabetic CLI patients treated with either peripheral or bone marrow derived CD34⁺ cells, noted no correlation was found between the numbers of injected CD34⁺ cells (as well as of the levels of any of the tested angiogenic cytokines) and clinical response (TcPO2 change) (Dubsky et al., Cell Transplant 2014; 23(12):1517-23; incorporated by reference in its entirety herein).

MSCs have been implicated in the treatment of a variety of conditions including CLI. In a double blind randomized placebo controlled Phase I/II trial, BM-MSCs were applied at the gastrocnemius muscle of the ischemic limb and demonstrated to be safe. Few efficacy parameters such as ABI and ankle pressure showed positive trends (Gupta et al., J Transl Med. 2013 Jun. 10; 11:143; incorporated by reference in its entirety herein), and it was hypothesized that this is not unexpected given that it is a single cell type which is cryopreserved, potentially thawed in uncontrolled processes having high viability loss, and injected in potentially uncontrolled methods.

Furthermore, TIE2-expressing monocytes/macrophages (TEMs) are highly angiogenic in tumors. In CLI patients, circulating TEM level were found 10-fold increased. Removal of the hypoxia reduced these levels to normal levels. In agreement, numbers of TEMs in ischemic muscle were twice those of normoxic muscle from the same patient (Patel et al., EMBO Mol Med. 2013 June; 5(6):858-69; Emanueli and Kränkel N. EMBO Mol Med. 2013 June; 5(6):802-4; both incorporated by reference in their entireties herein).

Given that multiple cellular components have been shown to positively affect vascularization, dosing for CLIRST III was proposed to be based on TNC/WBC counts rather than at the level of a particular cellular subpopulation (such as CD34⁺). Additionally, assuming that the pivotal trial data can show a stronger efficacy correlation of MNCs, it was hypothesized that a WBC surrogate is still practically sound since in previous experience of 280 bone marrow aspirates, WBC counts correlate to MNCs (R-Sq'd of 67.16%). A major practical advantage with this strategy is that WBC enumeration is rapid, reliable, and mobile therefore not requiring time-consuming point-of-care flow cytometry.

In contrast to the multicellular cultured product injected in the RESTORE-CLI (Aarstrom Biosciences) CLI trial (Powell et al., Mol Ther. 2012 June; 20(6):1280-6; incorporated by reference in its entirety herein), the aBMC product of the CLIRST III (Cesca Therapeutics) trial was comprised of both cellular and plasma components. In vitro bioactivity assays, migration and tube-formation, can indicate mechanistic effectiveness of a cellular product. Below, it was demonstrated that there is high bioactivity of the plasma fraction, thus strengthening the overall expected therapeutic composition of our product.

The purpose of the study was to assess the in vitro bioactivity of the biological product produced using the device. Additionally, a secondary objective is to evaluate using in vitro experiments the contribution (if any) individually or in combination among different components of the autologous Bone Marrow Concentrate (aBMC; also designation of the SurgWerks-CLI and VXP Device Output (“Product”)) on a surrogate. This example summarizes data obtained from aBMC, specifically aBMC plasma and aBMC white blood cell (WBC) fractions. Data presented include levels of cytokines, implicated in vessel formation from prior literature, in aBMC plasma and in WBC-conditioned media with their respective bioactivity as measured by Migration and Tube Formation assays using aBMC plasma and WBC-conditioned media as stimulants. Reported here are the results of a research study using aBMC obtained from 10 healthy donors processed with the SurgWerks-CLI and VXP System. aBMC was assayed in vitro for the ability to promote both HUVEC migration towards a chemotactic stimulus and formation of capillary-like HUVEC networks (“tubes”).

Equipment and Material Used in the Assays—

Biohazard safety cabinets; tissue culture incubator with humidity and gas control; sterile filters; Pipette-Aids and sterile pipettes (5 mL, 10 mL, 25 mL, and 50 mL); micro-pipettors with sterile disposable plastic tips; hemocytometer; Trypan Blue Solution (0.4%) (Sigma, Cat. No.: T8154); Routine light microscope and inverted microscope with CCD camera; Tubes (microtubes, and 5 mL, 15 mL and 50 mL); DPBS (Sigma, Cat. No.: D8662); Ethanol and sterile water; Fibronectin—liquid, from human plasma (Millipore, Cat. No. FC010); T75 flasks; EBM-2—Endothelial Basal Medium-2 (Lonza, Cat. No. CC-3156); EGM-2—Endothelial Growth Medium-2 BulletKit (Lonza, Cat. No. CC-3162); Trypsin-EDTA (Corning, Cat. No.: 25-053-CI); Bovine Serum Albumin, Delipidized (lyophilized) (Corning, Cat. No.: 354331); HUVECs, pooled donor, screened for angiogenesis markers and test positive for expression of eNOS, Ax1, Tie-2 and VEGFR2 (Lonza, Cat. No.: C2519AS); 3.0 um pore size transwell inserts and corresponding multiwell plates (BD Falcon HTS FluoroBlok 96-Multiwell Insert Plates, Corning, Cat. No.: 354148); Falcon HTS 96-Square Well, Flat Bottom Plates (Corning, Cat. No.: 353928); Matrigel® Matrix, Growth Factor Reduced (GFR) (Corning, Cat. No.: 354230); 96-well plates (TTP, Cat. No.: 92096); Hank's Balanced Salt Solution (HBSS) (Life Technologies, Cat. No.: 14025092); Calcein, AM (Ex/Em=495/516 nm) (Molecular Probes/Life Technologies, Cat. No.: C3100MP); Synergy 4 instrument—fluorescence microplate reader with bottom reading capability (BioTek, Serial Number: 217214); XE-2100™ Automated Hematology System (Sysmex, Sys. ID#476057); Amicon Ultra-15 Centrifugal Filter Unit with Ultracel-3 membrane (Millipore, Cat. No.: UFC900396); Human VEGF Quantikine ELISA Kits (R&D Systems, Cat. # DVE00); Human CXCL12/SDF-1 alpha Quantikine ELISA Kit (R&D Systems, Cat. # DSA00).

In vitro tube formation requires that endothelial cells, usually Human Umbilical Vein Endothelial Cells (HUVECs), migrate on Matrigel support and attach to one another following the encounter. Thereafter, cells can begin to form tubular structures. The initial migration is most likely a form of chemokinesis rather than chemotaxis since no relevant concentration gradients promoting directed cell migration are expected to be formed by initially homogeneously seeded cells. Unexpectedly, it was observed that compared to basal medium, aBMC cell-conditioned medium seemed to promote chemokinesis.

Test Articles (“Test Media”) were assessed for their potential to induce/support HUVEC migration and/or tube formation in Device Output (“Product”) plasma and in Conditioned media (CM) (Basal medium (EBM-2) supplemented with 0.1% BSA conditioned by aBMC cells blood).

Preparation of Conditioned Medium (CM).

CM was prepared by incubating RBC-lysed-washed WBC fraction in basal media (EBM-2+0.1% BSA) for 40-60 hours at 37° C., 5% CO2. CM was collected by centrifugation at 300×g (RCF) for 10 min. The supernatant was filtered using 0.22 microns filters ((Millex® GP 0.22 microns, Polyethersulfone (PES) membrane). Filtered CM samples were aliquoted and stored at negative 80° C. (below zero).

Concentrating Conditioned Medium.

To increase the concentrations of potential pro-angiogenic factors in CM, the CM samples were concentrated by centrifugation-based filtration using Amicon Ultra-15 Centrifugal Filter with Ultracel-3 membrane devices. In short, prior to use, membranes were briefly washed with ethanol to minimize the risk for microbial contamination in downstream application(s). For this, 70% ethanol was added to the membrane for approximately 1-2 minutes then removed. Thereafter, membranes were washed once with 10 mL of sterile water (water above the filter removed after incubation of approximately 5 minutes). Devices were then spun for 5 minutes at 3740 rpm (2.8K×g). Finally, approximately 4-5 mL of CM was added to each washed device, then devices spun at 3740 rpm (2.8K×g) for 40 minutes at 4° C. Volumes pre- and post-concentrating were measured. After concentrating, dilution factors were adjusted to 7 by supplementing higher concentrated samples with corresponding amounts of EBM-2 supplemented with 0.1% BSA. The BM samples used for the experiments are shown below in Table 8.

TABLE 8
BM samples utilized for this Bioactivity Assays

| Sample IDs | Sample Abbreviation |
|---|---|
| BM0008EV150205 | BM0008 |
| BM0009EV150205 | BM0009 |
| BM0010EV150209 | BM0010 |
| BM0011EV150209 | BM0011 |
| BM0012EV150211 | BM0012 |
| BM0013EV150211 | BM0013 |
| BM0014EV150216 | BM0014 |
| BM0015EV150216 | BM0015 |
| BM0016EV150219 | BM0016 |
| BM0017EV150219 | BM0017 |

Migration Assay.

HUVECs in basal medium (EBM-2+0.1% BSA) were seeded into insert wells with porous bottom membranes. Cells were quantified upon transmembrane migration towards the chemoattractant (Test Medium) in the bottom chamber. Two assays were conducted to study migratory assay (Method 1 & 2). In Method 1 assay, cells on the bottom side of the membranes were fixed and stained, then enumerated. Additionally, non-adherent cells in the Test Media were collected by centrifugation and counted. Total cell counts (adherent+non-adherent cells) were then used to assess the extent of the migratory effect. In the Method 2 assay, migrated cells were stained with calcein, AM, a compound enzymatically rendered fluorescent by live cells, then quantified using a fluorescence plate reader. Non-adherent cells were not quantified.

The latter method was implemented to optimize manpower and handling, and it greatly reduced hands-on time with a lower degree of technical variability. Furthermore, the new assay required lower volumes of test media thus being more compatible with test articles with very limited quantities. Shown below in Table 9, are the main functional differences between the two assay methods.

TABLE 9
Overview of the main functional differences between the two assays.

| | Method 1 | Method 2 |
|---|---|---|
| Format | 24-well | 96-well |
| Pore size | 8 micrometer | 3 micrometer |
| Test Medium volume | 650 microliters | 225 microliters |
| Cells quantified | Adherent cells on bottom side | Adherent cells on bottom side |
| | of porous membrane and | of porous membrane |
| | optionally non-adherent cells | |
| | in Test Media. | |
| Ease of use | Assay is difficult to conduct; | Assay is easy to conduct; a |
| | numerous steps with | full plate of up to 96 samples |
| | substantial potential for | can be processed feasibly |
| | disproportional cell loss; only | |
| | small number of samples can | |
| | feasibly be processed together. | |
| Expected | Poor, especially if cell counts | Good; assay does not require |
| reproducibility for | include counts of cells floating | post-migration cell handling |
| cell numbers at | in reservoir (below insert) | (washing and addition of |
| detection threshold | collected by centrifugation | calcein, AM does not require |
| | since removal of supernatant | cell detachment |
| | of miniscule non- sticky cell | |
| | pellets may remove large | |
| | percentage of cells | |
| Detection threshold | Potentially higher since | Potentially lower since very |
| | individual cells are counted | small numbers of cells may |
| | on the underside of membrane | not yield above-background |
| | | fluorescent signal. |

Cytokines Implicated in Neovascularization.

The levels of the following secreted factors were measured in the unprocessed (bone marrow aspirate), VXP-processed aBMC plasma and post-needle (TIN and SHN) samples, aBMC passed through the injection needles and the injectate collected: VEGF, SDF-1, and endostatin. VEGF and SDF-1 are considered pro-angiogenic factors, endostatin a negative regulator of angiogenesis.

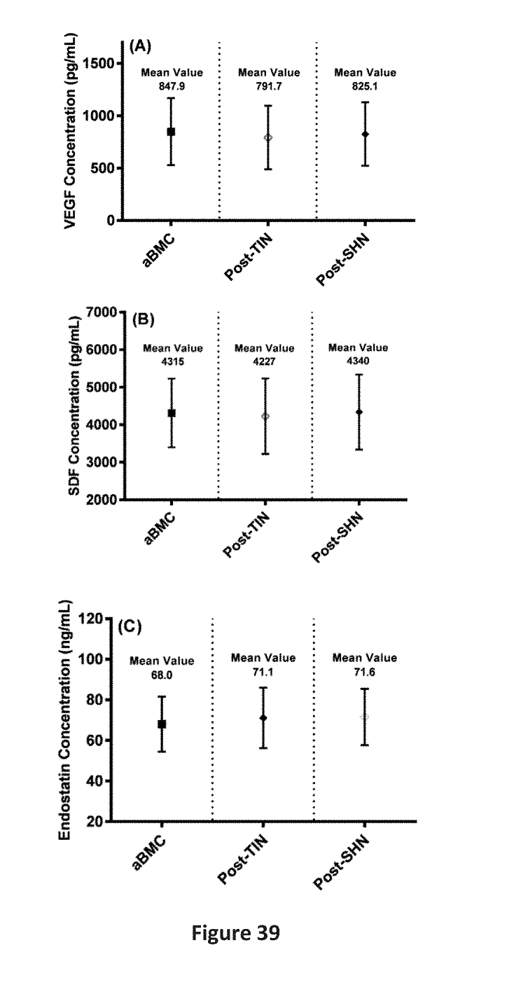

As shown in FIG. 39, VEGF and SDF-1 levels were not statistically different between VXP-processed aBMC plasma (pre-needle sample) and post-needle samples, TIN & SHN, suggesting that at a defined flow rate of 0.25 ml/s the cellular product is unaffected through the needle passage.

Migration Effects Induced by aBMC (“Product”) Plasma.

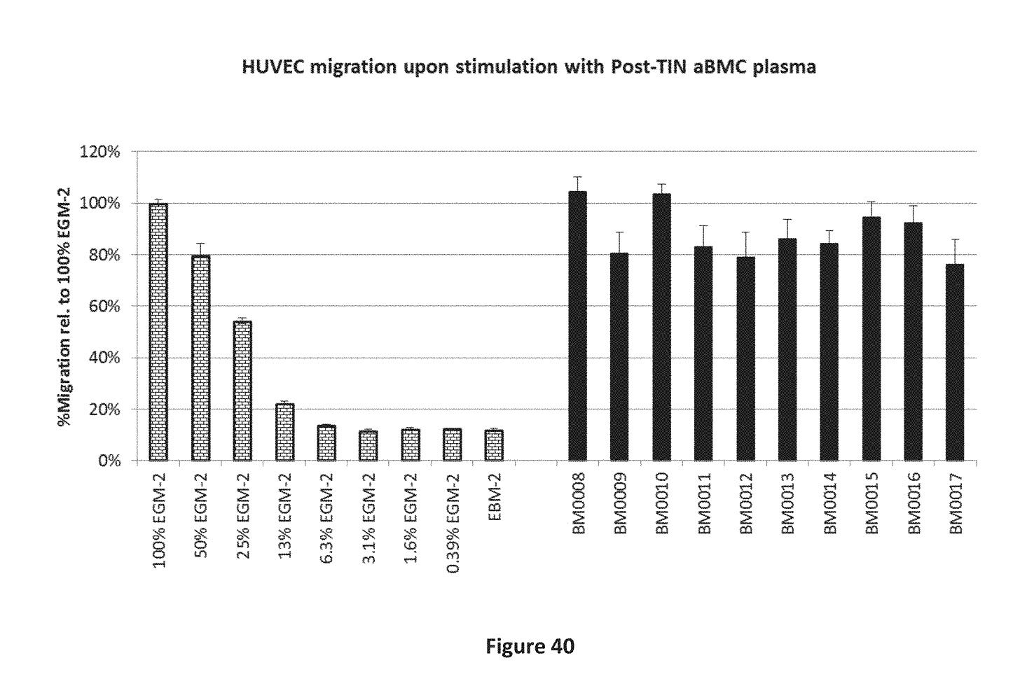

FIG. 40 shows that plasma from aBMC (passed through Therapeutic Infusion Needles (TINs)) 1:10 diluted with EBM-2 supplemented with 0.1% BSA potently induced HUVEC migration. EBM-2 with 0.1% BSA served as basal (negative) control, EGM-2 served as full endothelial medium (positive) control. Migration levels of various dilutions of EGM-2 with EBM-2 with 0.1% BSA demonstrate a positive correlation between the levels of pro-migratory factors.

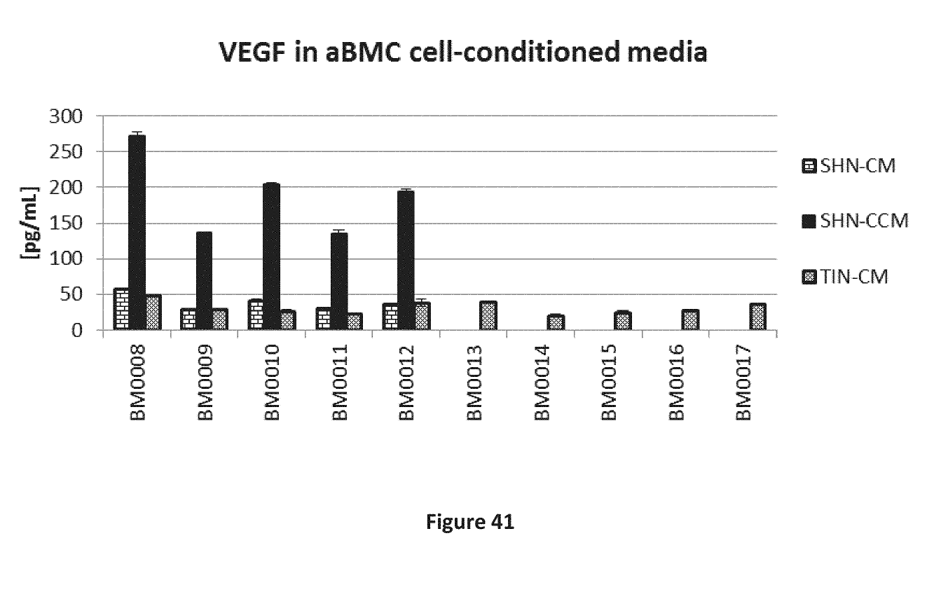

To assess whether and to what extent secreted factors from aBMC cells promote bioactivity and whether such factors would include VEGF and/or SDF-1, aBMC cell-conditioned media (CM) were assayed by ELISA and tested in Migration and Tube Formation assays. FIG. 41 demonstrates low levels of VEGF in unconcentrated CM, with substantial enrichment upon concentrating.

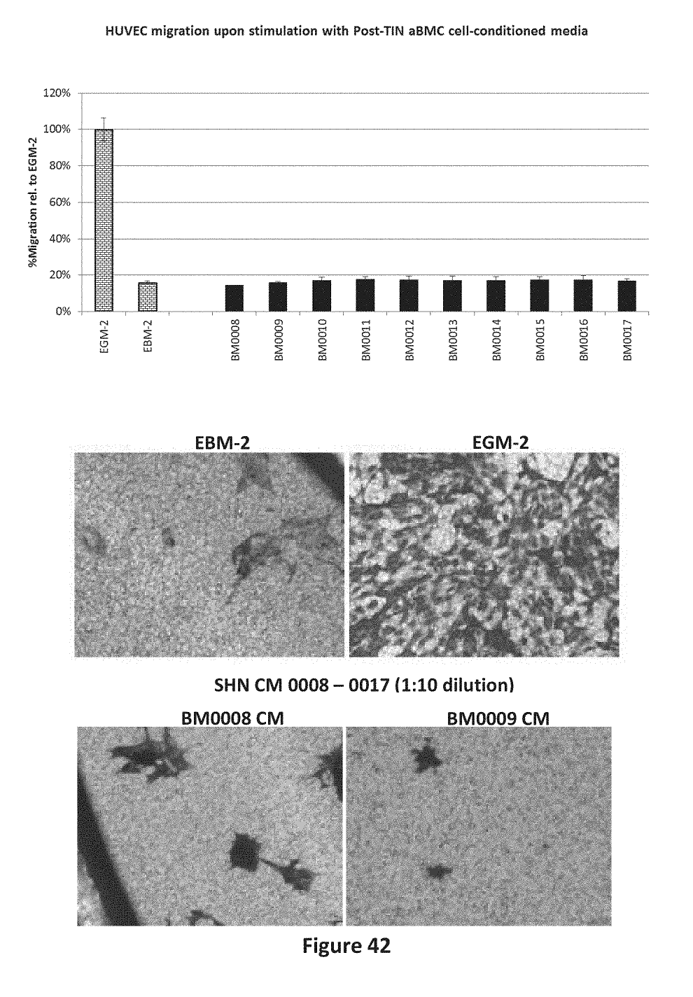

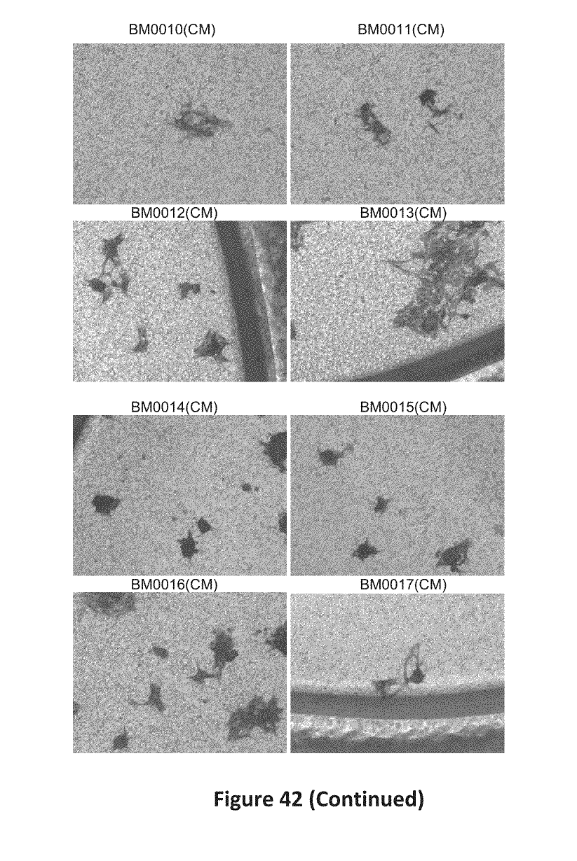

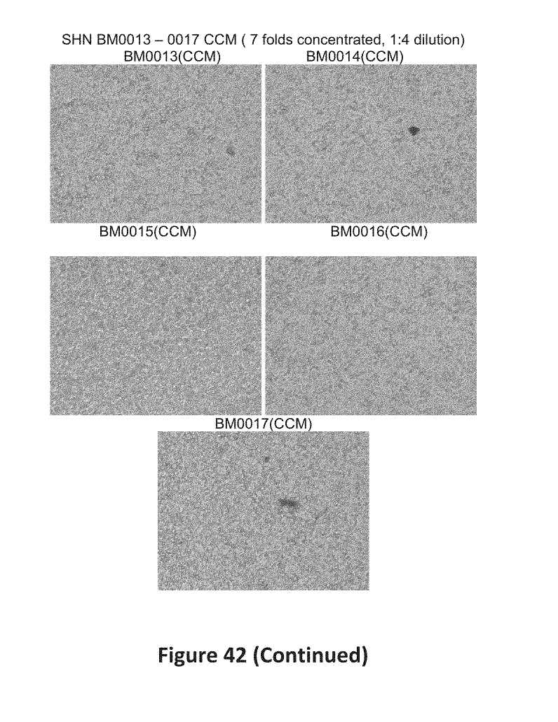

Migration effects following stimulation of HUVECs with EBM-2 with % BSA (basal medium) conditioned by cells from aBMC passed through TINs. FIG. 42 shows that conditioned medium did not induce HUVEC migration more extensively than the basal EBM-2 with 0.1% BSA. EBM-2 with 0.1% BSA served as basal (negative) control, EGM-2 served as full endothelial medium (positive) control.

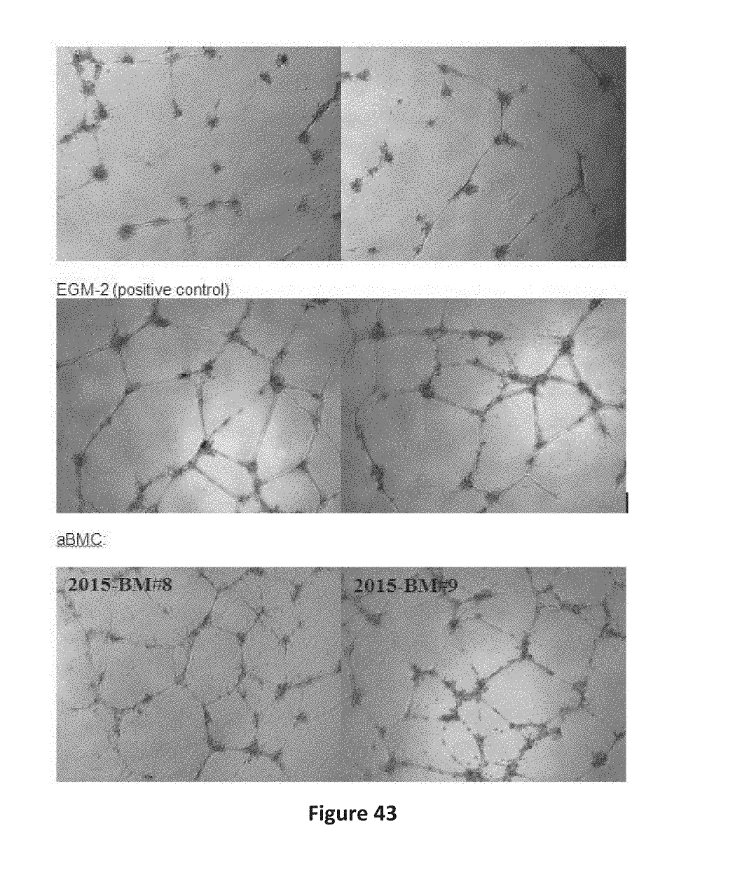

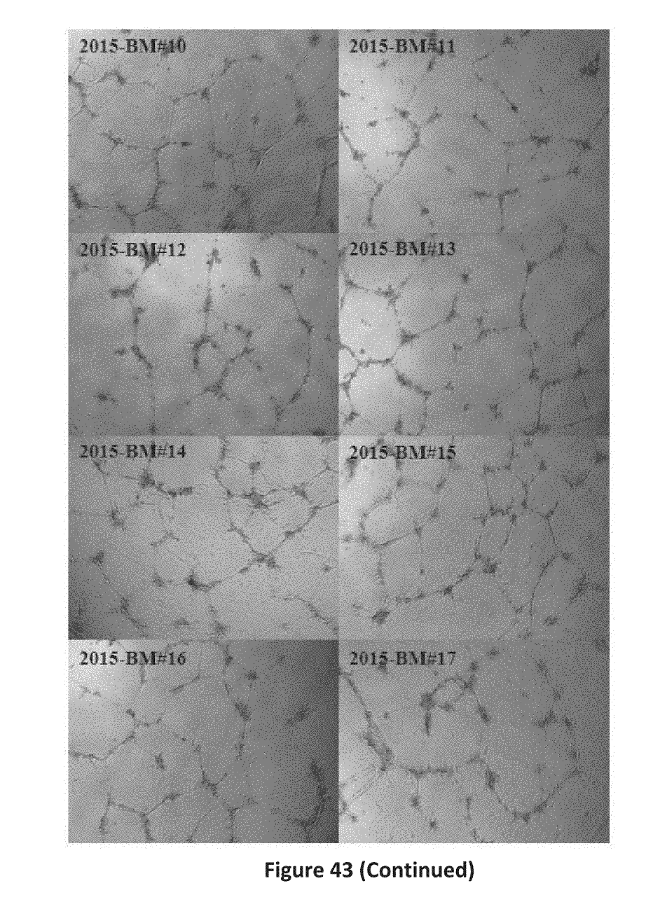

Tube formation induced by aBMC plasma. FIGS. 43 and 45-46 demonstrate that plasma from aBMC (passed though Therapeutic Infusion Needles (TINs)) 1:5 diluted with EBM-2 supplemented with 0.1% BSA potently induced HUVEC migration. EBM-2 with 0.1% BSA served as basal (negative) control, EGM-2 served as full endothelial medium (positive) control.

Quantification of Tube formation of the experiment represented by FIG. 43 using Image J (NIH). The “Tuning Functions Menu Tool” of the “Angiogenesis Analyzer for Image J” was used to measure a variety of analysis parameters related to angiogenesis. Described is a description of such main parameters which were used: (A) “Find a Tree” return a binary skeleton (or tree) of a natural HUVEC image acquired in phase contrast or fluorescence, (B) “Find & Remove Loops” removes the artifactual loops in a binary tree image on the criteria of the size, (C) “Find Extremities” detects the extremities in a binary tree. It returns results as overlays and binary map, (D) “Find Nodes and Junctions” detects nodes (pixels with 3 neighbors) as a circular dot and junctions, which correspond to nodes or group of fusing nodes, (E) “Find Nodes and Branches” Performs the same of the two precedent functions (detects extremities, nodes and junctions) plus other elements of the ramification: (1) segments; elements delimited by two junctions, (2) branches; elements delimited by a junction and one extremity, (3) twigs; a twig is a branch whose size is lower than a user defined threshold value and (4) isolated elements are binary lines which are not branched, and (F) “Record the Steps of Limbing” record in a dedicated repertory, the results of every iterations required to get an analysis corresponding to a limbing, including master segments, master junctions and meshes detection: (1) Master segments consist in pieces of tree delimited by two junctions none exclusively implicated with one branch, called master junctions, (2) Master junctions are junctions linking at least three master segments and optionally, two close master junctions can be fused into a unique master junction and (3) Meshes are areas enclosed by segments or master segments.

Detection of Constitutive Elements of the Network.

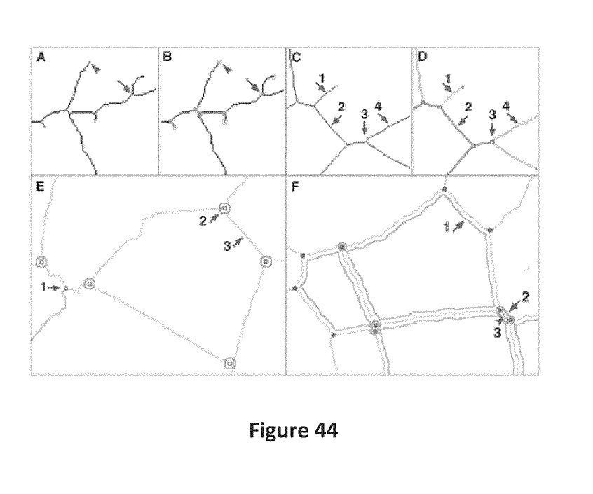

As shown in FIG. 44 extremities (arrow head A-B); nodes, identified as pixels that had at least 3 neighbors, correspond to a bifurcation (arrow A-B); twig (C1, D1), segment (C2, D2) delimited by two junctions (C3, D3) (note that this pointed junction is composed by several nodes) and branch (C4, D4). E shows a junction implicated only in branch (E1) and master junctions like E2 delimiting master segments (E3). F shows the master tree composed from master segments associated by master junctions delimiting the meshes (F1). Optionally, two close master junctions can be fused into a unique master junction (F2). Note the underlying segment (F3).

Quantification of Tube Formation.

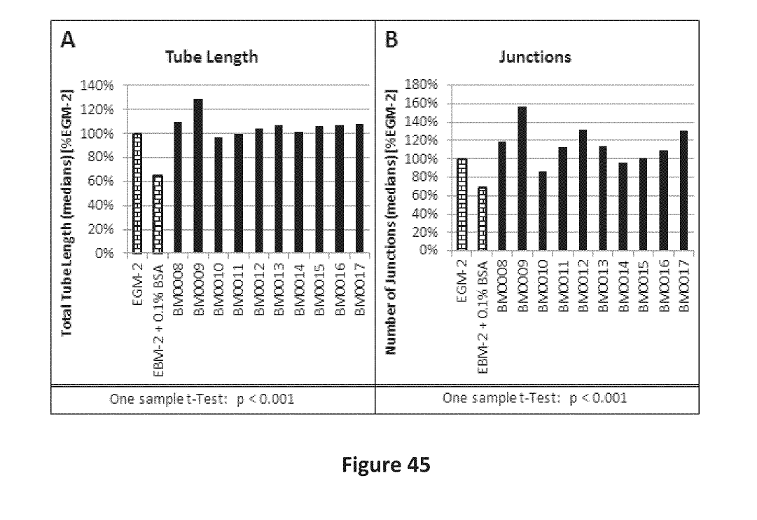

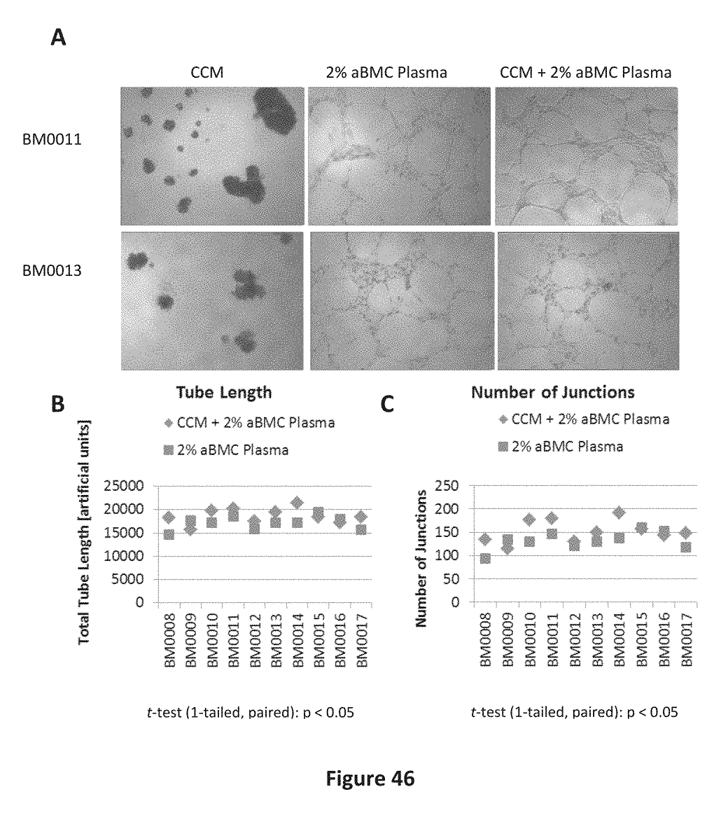

Using the Angiogenesis Analyzer from Image J (NIH), 3-4 images/sample of the above experiment (FIG. 43) were quantified. To minimize area selection bias, the median values were used for comparative assessments (FIG. 45). Statistical analysis (one sample t-Test) comparing the mean of the median values of each sample to the median value of the EBM-2 sample indicates that the total tube length and the number of junctions are significantly higher for aBMC plasma compared to EBM-2+0.1% BSA (p<0.001, FIG. 45). Tube formation following stimulation of HUVECs with basal medium (EBM-2 supplemented with 0.1% BSA) conditioned by cells from post-TIN aBMC. CM samples were concentrated using Amicon devices. FIG. 46A exemplifies that concentrated CM (CCM) induced HUVEC aggregation and that addition of 2% autologous Product plasma prevents this. Additionally, statistical analysis using paired t-tests suggests that the Total Tube Length (FIG. 46B) and the Number of Junctions (FIG. 46C) are statistically significantly (p<0.05) higher in CCM+2% aBMC Plasma samples compared to 2% aBMC Plasma-only samples.

The aBMC injectate contained both non-cellular (plasma) and cellular (aBMC cells) biological fractions. While the non-cellular contribution to the anticipated therapeutic effects likely results in local stimulation of both resident (stem) cells and aBMC cells, a likely transient effect, the cellular component itself relates to subpopulations of cells directly involved in the repair of the ischemic damage likely exerting an effect lasting longer than the one mediated by the plasma component. Chiefly involved in the latter type of effect can be, among others, endothelial progenitor cells (EPCs). Consistently, it was demonstrated in “potency” colony-forming unit (CFU) assays that SurgWerks-CLI and VXP-processed bone marrow concentrate traversed through the injection needle containing endothelial progenitor cells (EPCs; CFU-Hill assay), in addition to mesenchymal stem cells (MSCs; CFU-F assay) and hematopoietic progenitor cells (HPCs; CFU-H assay) (Report 620000).

In summary, this experiment outlined the bioactivity results for the following parameters and Test Media such as, for example, aBMC (“Product”) plasma promoted HUVEC migration and Tube formation. Consistent with their roles in vascularization, VEGF and SDF-1 were detected at notable levels in preprocessed Bone Marrow Aspirate (BMA) and aBMC plasma and, in the case of VEGF, also in aBMC cell-conditioned medium although at much lower levels. Unexpectedly, levels of both cytokines were significantly higher in aBMC plasma than in BMA plasma, suggesting either the VXP System processed output has had a concentration effect on SDF-1 or VEGF or the close proximity of nucleated cells after removal of significant quantity of RBCs, i.e., higher number of cells in a small volume, enhances cytokine secretion. Endostatin levels did not differ significantly between BMA and aBMC. There was no effect of needle passage on cytokines or cells. Additionally, aBMC cell-conditioned media (CM) did not promote angiogenesis. However, concentrating the aBMC CM consistently appeared to have promoted HUVEC cell aggregation on Matrigel, a procedure potentially involving chemokinesis.

Given the lack of obvious concentration gradients of chemotactic factors in the static environment of the Tube formation assay, promotion of HUVEC cell aggregation is likely due to upregulated chemokinesis, i.e., random/non-directional migration. Several factors have been implicated in promoting chemokinesis, including SDF-1, VEGF, and FGF-2 (bFGF): While SDF-1 exerts chemokinetic (and chemotactic) activity (Hattori et al., Blood. 2001 Jun. 1; 97(11):3354-60; incorporated by reference in its entirety herein), VEGF may down-regulate chemokinesis (Barkefors et al., J Biol Chem. 2008 May 16; 283(20):13905-12; incorporated by reference in its entirety herein) but was shown to up-regulate FGF-2, a chemokinesis-stimulating factor (Masaki et al., Circ Res. 2002 May 17; 90(9):966-7; Seghezzi et al., J Cell Biol. 1998 Jun. 29; 141(7):1659-73; both incorporated by reference in their entirety herein). Taken together, evidence is provided that aBMC cell-conditioned media contain factors promoting chemokinesis and thus potentially a first step in the tube formation process, however, the underlying mechanism remains elusive.

The data presented for these set of experiments are consistent with the aBMC injectate containing a fraction of plasma inductive for cell migration and supports the following preliminary mechanisms of action. Firstly, product plasma promotes migration of both aBMC cells and tissue resident cells such as progenitor cells including EPCs or differentiated endothelial cells. Since the vasculature in the ischemic regions is impaired, one may envision a model in which, for instance, circulating EPCs with native homing potential to such regions are first recruited into the vicinity of the aBMC injection sites where they receive growth factor support by the aBMC plasma enabling their replication and further migration towards the cores of the ischemic regions. A limitation of this in vitro study is the use of bone marrow samples from healthy donors compared to the subjects fulfilling the enrollment criteria for CLIRST III.

### Example 8: Results of a Compassionate Use Human Clinical Study Conducted to Test the Ability of Autologous Bone Marrow Concentrate (aBMC) to Improve Wound Healing, Rest Pain and Amputation Free Rate in Critical Limb Ischemia (Peripheral Arterial Disease, PAD) Patients

As discussed previously, autologous cell therapies are patient-specific due to biologically active cellular and acellular components. In vitro bioactivity assays can only indicate limited mechanistic effectiveness of a cellular product. The complex mechanistic action of acellular and cellular product i.e., aBMC product, with the hypoxic tissue can be best studied in patients suffering from peripheral artery disease. In this set of experiments, the treatment data for six (6) “no option” Critical Limb Ischemia (CLI) patients who presented with Rutherford categories 4, 5 and 6 at baseline, having mean age of 50.83 years and failed all prior standard revascularization therapies is presented. These “poor” or “no option” patients were treated with autologous bone marrow cell concentrate (“aBMC”) prepared using Cesca's SurgWerks system under the surgeon's supervision in the operating room. The patients were followed up for 6 months.

The device cellular output was tested for sterility, cellularity, and cell viability. Each patient had 120 mL of bone marrow harvested inclusive of anticoagulant, which was immediately processed at the point-of-care in the operating room. The prepared aBMC was transferred aseptically back into the sterile field and injected intramuscularly into multiple sites of the afflicted limb of the CLI patients. All patients tolerated the procedure well.

Adverse/Serious Adverse Events (AE/SAEs).

An adverse event is any untoward treatment effect on a patient, including any unfavorable and any unintended sign (including an abnormal laboratory finding), symptom, or disease during the aBMC harvest and implantation, whether or not considered related to it. Of the 6 treated patients, one (1) patient underwent major limb amputation at 4 months post aBMC treatment, which was considered by the treating physician as unrelated to the treatment and a natural course of disease progression. There were no other adverse events or serious adverse events reported for the patient population at the time of treatment and during the 6 months post aBMC administration.

Major Amputation Free Survival.

Five (5) out of six (6) patients survived at 6 months, without major limb amputation, indicating an 83.3% major limb amputation free survival. One (1) patient underwent major limb amputation (above the knee) at 4 months following aBMC treatment due to disease progression and development of gangrene in the afflicted limb.

Rest Pain.

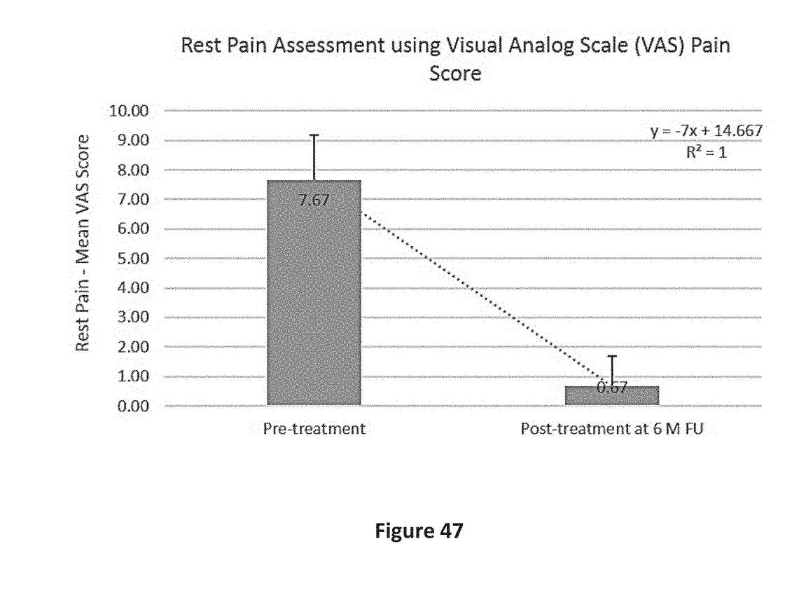

Rest pain assessment was performed through Visual Analog Scale (VAS) testing, which uses a psychometric (self-report) response scale scored between 0 to 10 (0=No pain/hurt, 2=Hurts a little/pain is mild, 4=Hurts a little more/pain is causing discomfort, 6=Hurts even more/pain is distressing, 8=Hurts a whole lot/pain is horrible, 10=Hurts worse/pain is excruciating) at intra-operative day/baseline and at 6 months. The mean rest pain score at baseline was 7.67, indicating that the majority of the patients had severe pain at rest. At the post-treatment 6 month follow-up visit, all available patients showed significant improvement, where the mean rest pain score was 0.67, with four out of six patients experienced no pain at rest. Overall improvement in rest pain and quality of life (QOL) at 6 months follow-up was statistically significant compared to baseline. FIG. 47 shows the pre-treatment and post 6-month follow-up data.

Ulcer, Gangrene and Other Skin Changes.

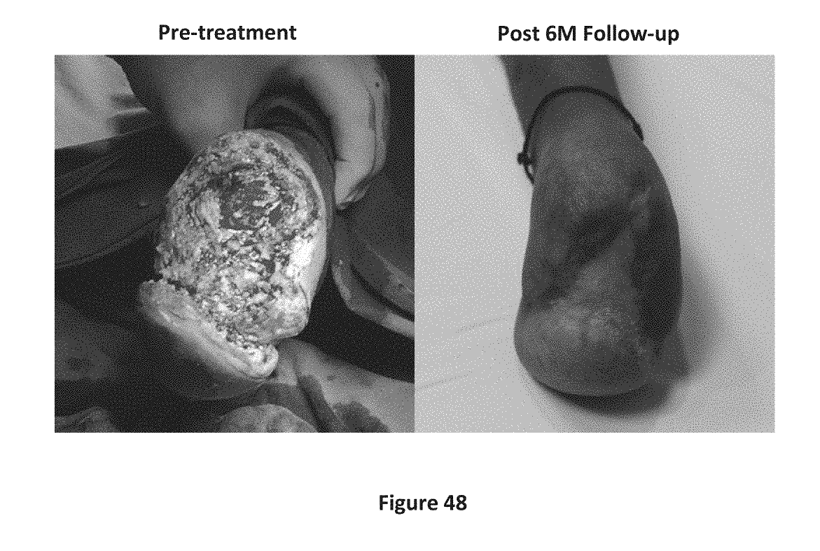

Evaluation of the integument for ulceration, gangrene and other skin changes in the affected limb was performed at intra-operative day/baseline during aBMC implantation and at 6 months. The treating physician evaluated the ulceration and gangrene in the afflicted limb of the patients by visual clinical inspection. All findings were clinically correlated. At baseline, 5 of 6 patients were suffering from foot ulcers or open wounds. At the 6 month follow-up, none of the patients presented with ulcers or gangrene or open wounds. As a result, 6 months after the treatment with aBMC, all ulcers or open wounds had healed and patients showed significant improvement (wound healing) in the diseased condition and quality of life. FIG. 48 shows the pre-treatment and post 6-month follow-up images of a patient, as a representative example.

Sterility Analysis.

A small aliquot of bone marrow concentrate (aBMC) was collected and tested for sterility at multiple steps after VXP processing and after traversing through the fluid path, including the infusion needles. Results showed that all 6 sample aspirates (post-trocar) and concentrates (post-processing) were negative (no growth) per USP <71> 14 day culture method and no bacterial or fungal elements were seen in the rapid Gram's stain smears.

In summary, preliminary evidence is presented that show that the CLIRST aBMCs contain a variety of components having biological activity in models simulating vasculogenesis and this evidence can be verified in a pivotal study for contributing to the repair of ischemic, damaged, tissues in a Rutherford 5 CLI subject.

All publications, patents, and patent applications mentioned in this specification are herein incorporated by reference for all purposes to the same extent as if each individual publication, patent, or patent application was specifically and individually indicated to be incorporated by reference.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of ameliorating or inhibiting critical limb ischemia or a condition associated with critical limb ischemia in a subject comprising:
identifying a subject having a peripheral vascular disease, peripheral arterial disease, critical limb ischemia or a condition associated with critical limb ischemia; and
administering intramuscularly to said subject a composition comprising:
(a) a cell population, wherein the cell population comprises bone marrow total nucleated cells and red blood cells,
(b) an anticoagulant, and
(c) autologous plasma,
wherein the composition has a viscosity of 1.5 to 5.0 centipoise (cP) measured at 37° C.,
wherein the composition comprises a viable cell dose and, wherein said composition is administered through a standard terminally-ported cannula needle, or a cannula side-ported needle or catheter comprising a plurality of ports and a closed end.

2. The method of claim 1, wherein said cell population comprises 2%, 3%, 4%, 5%, 6%, 7%, 8%, 9%, 10%, 11%, 12%, 13%, 14%, 15%, 16%, 17%, 18%, 19%, or 20% packed total cell fraction of the composition (total cell volume of the composition) or red blood cell fraction of the composition or an amount of total cells or red blood cells that is within a range defined by any two of the aforementioned values.

3. The method of claim 1, wherein the cell population comprises at least 1×10⁸ total nucleated cells (TNCs), and wherein the cell population is processed from 120-180 mL autologous bone marrow aspirate and is concentrated for a final 20 mL volume as enumerated intra-operatively using a diagnostic instrument to guide the aspiration of autologous bone marrow, wherein the cells comprise:
(a) a cell viability ≥70% and
(b) a white blood cell recovery ≥80%.

4. (canceled)

5. The method of claim 1, wherein the cell population comprises stromal cells.

6. The method of claim 5, wherein the stromal cells are lineage negative/dim, CD45 negative/dim and CD73 positive cells.

7-8. (canceled)

9. The method of claim 1, wherein the viable cell dose given to the subject as measured after the bone marrow aspiration and after the bedside processing but prior to injection at the point of care is 1.75×10⁷ to 7.67×10⁷ white blood cells per mL, 3.65×10⁶ to 2.15×10⁷ mononuclear cells per mL, or 5.0×10³ to 20.0×10³ colony forming units (CFU-H) per mL.

10-11. (canceled)

12. The method of claim 1, wherein said standard terminally-ported cannula needle, cannula side-ported needle or catheter has a lumen size (diameter) of 0.33-0.69 mm.

13-14. (canceled)

15. The method of claim 1, wherein said cannula side-ported needle or catheter comprises 10-24 side ports.

16. The method of claim 15, wherein said side ports have a diameter of 0.46 mm to about 0.56 mm.

17. The method of claim 1, wherein said composition is administered at a rate of 0.1 mL to 0.5 mL per second.

18. (canceled)

19. The method of claim 1, wherein said administered composition has a muscle distribution area of 4-6 cm².

20. The method of claim 1, wherein said composition is administered at a pressure of 3-5 psi.

21. The method of claim 1, wherein said composition is administered to said subject intramuscularly at a shear stress of less than or equal to 400 Pa.

22-30. (canceled)

31. A method of ameliorating or inhibiting critical limb ischemia or a condition associated with critical limb ischemia in a subject comprising:
identifying a subject having a peripheral vascular disease, peripheral arterial disease,
critical limb ischemia or a condition associated with critical limb ischemia; and
administering intramuscularly or intradermally to said subject a composition comprising a cell population that comprises autologous bone marrow mononuclear cells and red blood cells, wherein said composition is aspirated, concentrated, and administered to said subject using a standard terminally-ported cannula needle, or a cannula side-ported needle or catheter comprising a plurality of ports and a closed end.

32. The method of claim 31, wherein the cannula side-ported needle or catheter has a lumen size (diameter) of 0.33-0.69 mm.

33-34. (canceled)

35. The method of claim 31, wherein said cannula side-ported needle or catheter comprises 10-24 side ports with a diameter of about 0.46 mm to about 0.56 mm.

36. (canceled)

37. The method of claim 31, wherein said composition is administered to said subject at 0.25 mL to 0.5 mL per dose.

38. (canceled)

39. The method of claim 31, wherein said composition is administered in at least 30 doses.

40. (canceled)

41. The method of claim 31, wherein said composition is administered in a single dose per day on each of multiple days.

42. The method of claim 31, wherein said composition is administered in at least two doses per day on each of multiple days.

43. The method of claim 31, further comprising measuring in said subject an increased blood flow, an increase in transcutaneous oxygen, an increase in angiogenesis, an increase in vascularity, an increase in perfusion, an improvement in wound healing, an increase in limb salvage, or an increase in amputation-free survival rate after administration of said composition.

44-46. (canceled)
